(19) United States
(12) Patent Application Publication
(10) Pub. No.: US 2008/0112929 A1
(43) Pub. Date: May 15, 2008

(54) SHIELDED ADENOVIRAL VECTORS AND METHODS OF USE

(76) Inventors: Imre Kovesdi, Rockville, MD (US); Susan J. Hedley, Birmingham, AL (US); Nikolay Korokhov, Gaithersburg, MD (US)

Correspondence Address: FROMMER LAWRENCE & HAUG, 745 FIFTH AVENUE- 10TH FL., NEW YORK, NY, 10151, US

(21) Appl. No.: 11/947,771

(22) Filed: Nov. 30, 2007

Related U.S. Application Data
(63) Continuation-in-part of application No. PCT/US06/21204, filed on May 31, 2006.
(60) Provisional application No. 60/685,960, filed on May 31, 2005.
(60) Provisional application No. 60/725,481, filed on Oct. 11, 2005.
(60) Provisional application No. 60/748,416, filed on Dec. 8, 2005.

(51) Int. Cl. A61K 48/00; C12N 15/861; C07K 14/075

(52) U.S. Cl. 424/93.2; 435/456; 530/350

(57) ABSTRACT

The present invention encompasses replication deficient or a replication competent adenoviral vectors which may comprise moieties covering and shielding the vector from the effects of humoral immune responses, as well as a method of constructing and using such vectors. The preferred viral constructs may incorporate the shielding moieties into the pIX coat protein of the adenovirus vectors. The invention also provides recombinant viral vectors with both shielding and specific targeting abilities. Preferably, the viral vector may comprise a nucleic acid sequence, which codes for therapeutically important genes. Methods for treating of a host with an effective amount of adenovirus vector of the present invention are also provided.

### INCORPORATION BY REFERENCE

This application is a continuation-in-part application of international patent application Serial No. PCT/US06/21204 filed May 31, 2006, which published as PCT Publication No. WO 2007/050128 on May 3, 2007, which claims priority to U.S. provisional patent application Ser. Nos. 60/685,960 filed May 31, 2005; 60/725,481 filed Oct. 11, 2005 and 60/748,416 filed Dec. 8, 2005.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FEDERAL FUNDING LEGEND

This invention was supported in part using federal funds from the National Institutes of Health. Accordingly, the Federal Government has certain rights in this invention.

### FIELD OF THE INVENTION

This invention pertains to replication deficient or a replication competent adenoviral vectors comprising moieties covering and shielding the vector from the effects of humoral immune responses, as well as a method of constructing and using such vectors.

### BACKGROUND OF THE INVENTION

Adenovirus (Ad) vectors are employed in a wide range of gene therapy and vaccine applications. Development of these vectors in the clinical context has highlighted that the host humoral response may limit therapeutic utility due to pre-existing titers of neutralizing antibodies within the human population, particularly against the commonly used Ad5 and Ad2 serotype vectors due to the general exposure to Ads. Attempted methods to overcome this have included using chimeric Ad vectors expressing capsid proteins from several different adenoviral serotypes or the use of different serotypes, in particular Ad11 and Ad35, to which the human population has a lower prevalence of neutralizing antibodies. It is still perceived that with all these approaches the development of humoral and cellular immune responses eventually occur. However, the process of biochemical modification of the capsid might prove fruitful. On this basis the utilization of a shielding molecule to coat the adenovirus capsid would enable the vector to evade the host immune system.

This concept of host immune evasion has been realized by the use of chemical conjugation of bi-functional polymers, such as polyethylene glycol (PEG), to the Ad capsid (Croyle et al. 2001, J. Virol. 75:4792-4801; Croyle et al. 2002, Hum. Gene Ther. 13:1887-1900). Despite the success of the proof of principle vector studies, chemical cross-linking of PEG would be problematic in clinical translation. In addition to heterogeneity of composition, due to the randomness of the cross-linking, batch-to-batch variation in potency is seen. These issues represent significant problems with respect to scale up and regulatory approval. An alternative means to attach targeting and shielding proteins is therefore required.

The possibility of coating Ad with polymers (pc-Ad) was demonstrated recently (Green and Seymour, 2002, Cancer Gene Ther. 9:1036-1042). Apparently, the pc-Ad persists substantially longer in the blood circulation than Ad, allowing the possible accumulation of the particles in the tumor sites. However, this methodology eliminates shielding the vector by genetically incorporated sequences. Although, different targeting molecules were successfully incorporated into the system the separate production of proteins and/or antibodies might be prohibitively expensive. Furthermore, this method could not be used for replicating vectors as once the virus replicates, the virus progeny loses its ability to be shielded and is subject to the same immunological constraints as a non-coated vector.

A key technological advancement towards generating a shielded Ad vector is the definition of an optimal capsid locale to incorporate the shielding moiety. There are several location exists on the adenovirus capsid that a shielding moiety can be incorporated. Insertions of peptides, protein fragments and proteins have been incorporated into the fiber (e.g. Wickham et al. 1996, Nat. Biotech. 14:1570-1573; Wickham et al. 1997, J. Virol. 71:8221-8229; Dmitriev et al. 1998, J. Virol. 72:9706-9713; Xia et al. 2000, J. Virol. 74:11359-11366; Mizuguchi et al. 2001, Gene Ther. 8:730-735; Nicklin et al. 2001, Mol. Ther. 4:534-542; Belousova et al. 2002, J. Virol. 76:8621-8631), penton base (Einfeld et al. 1999, J. Virol. 73:9130-9136) and hexon (Vigne et al. 1999, J. Virol. 73:5156-5161; Worgall et al. 2005, J. Clin. Invest. 115:1281-1289; Wu et al. 2005, J. Virol. 79:3382-3390). Recent work has identified the minor capsid protein, pIX, as the preferred insert ional location (Dmitriev et al. 2002, J. Virol. 76:6893-6899) for embodying such utility. The pIX capsid protein of Ad allows the genetic incorporation of motifs, in a range of size and complexity. Additionally, pIX protein is of a defined and high copy number in the capsid and would therefore guarantee a high valency and uniformity of clinical grade vector. The C-terminus of pIX has an alpha helical structure that doubles as a “zipper” between groups of seven (hexons). The very terminal part is exposed and can be modified using spacer sequences (Velling a et al. 2004, J. Virol. 78:3470-3479; Velling a et al. 2005, J. Virol. 79:3206-3210).

In regards to motifs that pIX can incorporate, these include large proteins such as green fluorescent protein (GFP) (Le et al. 2004, Mol. Imaging. 3:105-116; Meulenbroek et al. 2004, Mol. Ther. 9; 617-624), motifs allowing additional proteins to be attached, e.g. biotin acceptor peptide (Campos et al. 2004, Mol. Ther. 9:942-954), as well as small peptide motifs, e.g. polylysine (Dmitriev et al. 2002, J. Virol. 76:6893-6899). The first class of molecules could include GFP, RFP and albumin, for example and these would provide a general shielding effect, but could also include antibody-related fragments, e.g. single chain antibodies or single domain antibodies that would provide dual functions (both targeting and shielding). A limitation of Ad vectors is their broad tropism, and it is widely recognized that targeting of the Ad vector would improve clinical utility of these vectors.

Adenovirus vectors have limitations due to pre-existing neutralizing antibodies present in the human population. Effective repeat administration of Ad vectors to most tissues is hindered by a strong neutralizing antibody response to the vector. Skeletal muscle was one of the few tissues where repeat Ad vector administration was successfully demonstrated (Chen et al. 2000, Gene Ther. 7:587-595) However, the success of this procedure was highly dependent on the initial dose of Ad used in the experiment. Therefore, it is expected that repeat dosing in human is problematic. The development of a uniform shielding method in which Ad function of gene delivery in vivo is maintained is critical.

Bacteria express surface proteins that interact with human extracellular proteins. There is a large number of albumin and antibody (Ab) binding proteins existing in nature. Some of these molecules like Protein A, Protein G, Protein PAB are well characterized (Johansson et al., (2002) J Biol Chem, 277(10), 8114-8120). Pathogens use these proteins to avoid detection by the human immune system. Mutated and modified versions of these sequences have been incorporated into adenoviral vectors to enable targeting specific receptors (Korokhov et al., (2003). J Virol, 77(24), 12931-12940). However, these methods have not been previously proposed to shield a vector from Ab responses.

There remains a need for adenoviral vectors that have greater flexibility in their delivery and use, and can provide greater success in the treatment of a tumor, cancer, and vascular or genetic diseases, as well as vectors for vaccines for the treatment and prevention of infectious diseases. The present invention provides such vectors, as well as a method of constructing such vectors, and a therapeutic method involving the use of such vectors.

Citation or identification of any document in this application is not an admission that such document is available as prior art to the present invention.

### SUMMARY OF THE INVENTION

The present invention provides for a chimeric protein adenoviral protein, which may comprise a non-native amino acid sequence, wherein the non-native amino acid sequence may be a shield for the adenovirus (Ad) vector from humoral immunity. The shielding moiety may also serve as a ligand that binds to a substrate present on the surface of cells.

This invention pertains to viral or non-viral vectors, but most specifically replication deficient or a replication competent adenoviral vectors. These vectors comprising moieties covering and shielding the vector from the effects of humoral immune responses by methods described below. Small albumin and/or antibody binding proteins may be incorporated into the adenoviral capsid. These peptides may be incorporated into the fiber (e.g., C-terminus, HI loop, etc.), hexon (e.g., loop 1, loop 2, etc.), penton base (e.g., close to or replacing the RGD domain, etc.), pIX (e.g., C-terminal), pIII (e.g., N-terminal) or any combination thereof.

Once the described vectors are constructed, they may be incubated in vitro with shielding moieties, such as human serum albumin (HSA) or antibodies. The vectors may be injected also directly in vivo to an animal or preferably a human without pre-incubation. The vector in this case may be coated with self proteins of the individual animal or person avoiding complications of transferring a foreign substance. The coated vector may have a longer circulation time in the body, providing sufficient time to reach its target (e.g. metastasis or cancer cell, target organ etc.) Furthermore, the coated vector may stay in the circulatory system longer than the uncoated vector even in the presence of antibodies against the viral coat proteins. It is also possible to multiply dose such vectors for improved efficacy.

In an advantageous embodiment, the adenoviral protein used to anchor the shield is protein IX (pIX). The chimeric pIX protein may contain an adenoviral pIX domain and also a non-native amino acid, wherein the non-native amino acid is a shield protecting the Ad vector from humoral immunity. Furthermore, the shielding moiety may also serve as a ligand that binds to a substrate present on the surface of cells. Hence the chimeric pIX can be used to target vectors containing such proteins to desired cell types. Thus, the invention provides vector systems including such chimeric pIX proteins, as well as methods of protecting Ad vectors from humoral immunity and infecting cells using such vector systems.

The chimeric proteins may be advantageously chimeras of the “minor” capsid proteins, pIIIa and pIX of adenovirus. Proteins pIIIa and pIX are present on the adenoviral capsid as monomers and trimers, respectively, and the proteins have an extended amino-terminus and carboxy-terminus parts, respectively. Thus, both locale and structural considerations indicate that pIIIa and pIX are the ideal capsid proteins for incorporating shielding peptides and achieving genetic modification to shield and/or retarget of the adenovirus. The minor capsid protein pIIIa gene may be modified by inserting a DNA sequence encoding a stabilized antibody into the 5′ end of the pIIIa gene, resulting in a stabilized antibody inserted at the N terminus of the pIII protein. Similarly, the minor capsid protein pIX gene may be modified by inserting a DNA sequence encoding a single chain antibody into the 3′ end of the pIX gene, resulting in a stabilized antibody inserted at the C terminus of the pIX protein. In another embodiment, the chimeric adenoviral proteins may be derived from a fiber, a penton, a hexon protein or a protein VI.

The non-native amino acid sequence may be a shield for the adenovirus (Ad) vector from humoral immunity. The non-native amino acid sequence may encode a self protein, serum protein, an albumin related protein, an alpha 1 antitripsin related protein or a single chain antibody. In an advantageous embodiment, the non-native amino acid sequence may encode a ligand, wherein in an advantageous embodiment, the ligand binds to a substrate present on the surface of the cell. The ligand may recognize a CD40 protein or the ligand may be an RGD-containing or polylysine-containing sequence. In another embodiment, the non-native amino acid sequence may be constrained by a peptide loop within the chimeric protein. Advantageously, the peptide loop may comprise a disulfide bond between non-adjacent amino acids of the proteins.

The present invention also relates to adenoviral capsids, preferably an adenoviral capsid which may comprise any one or more of the above-described chimeric proteins. In one embodiment, the adenoviral capsid may bind dendritic cells. In another embodiment, the adenoviral capsid may comprise a mutant adenoviral cellular receptor, wherein the mutant adenoviral cellular receptor may have an affinity for a native adenoviral cellular receptor of at least about an order of magnitude less than a wild-type adenoviral fiber protein. The adenoviral capsid may comprise an adenoviral penton base protein having a mutation affecting at least one native RGD sequence and/or at least one native highly variable region (HVR) sequence in the hexon. In another embodiment, the adenoviral capsid may lack a native glycosylation or phosphorylation site. In a preferred embodiment, the adenoviral capsid may elicit less immunogenicity in a host animal as compared to a wild-type adenovirus. In a more preferred embodiment, the adenoviral capsid may elicit at least 50% less immunogenicity in a host animal as compared to a wild-type adenovirus. In another embodiment, the adenoviral capsid may comprise a second non-adenoviral ligand advantageously conjugated to a fiber, a penton, a hexon, a protein IIIa or a protein VI or any combinations thereof. In yet another embodiment, the non-native amino acid of the adenoviral capsid may comprise a ligand and a second non-adenoviral ligand recognizes the same substrate as the non-native amino acid. In an advantageous embodiment, the adenovirus is a conditionally replicating vector (CRAd), AdΔ24S-RGD, that may comprise of an adenoviral capsid incorporating a shielding moiety into the pIX protein C-terminal domain as well as an having an RGD containing peptide inserted into the fiber HI loop for enhanced transduction of clinically relevant cells and tissues.

The invention also encompasses viral vectors, preferably an adenoviral vector comprising the adenovirus of described herein. The adenoviral vector may comprise any one or more of the adenoviral capsids described above. The adenoviral vector may be replication competent, replication deficient or replication incompetent. In another embodiment, the adenoviral vector may not productively infect human embryonic kidney cells, advantageously HEK-293 cells. In another embodiment, the adenoviral vector may comprise an adenoviral genome, which may comprise a non-native nucleic acid for transcription. In another embodiment, the adenoviral vector may comprise an adenoviral genome, which may comprise a non-native nucleic acid for maintaining efficient packaging size of the Ad genome.

In one embodiment, adenovirus may be operatively linked to a non-viral promoter, advantageously a non-adenoviral promoter. The non-viral promoter may be a cell-specific promoter, a tissue-specific promoter or a regulatable promoter. In yet another embodiment, the non-viral promoter is operably linked to a non-native nucleic acid for transcription.

The invention also provides for transformed host cells comprising such vectors. In one embodiment, the vector may be introduced into the cell by transfection, electroporation, transformation or infection. The invention also provides for a method for producing the Ad vectors in a transformed cell expressing the adenovirus of the present invention which may comprise transfecting, electroporating, transforming, contacting or infecting a transformed host cell with the adenovirus to produce a transformed host cell and maintaining the transformed host cell under biological conditions sufficient for expression of the adenovirus in the host cell.

The invention encompasses a method for administrating the adenovirus of the present invention to a subject in need thereof, advantageously at least twice, which may comprise administering to the subject in need thereof a therapeutically effective amount of the adenovirus described herein wherein the non-native amino acid shields the vector from humoral responses and/or targets a tumor cell such that the adenovirus infects the target cells. The invention also encompasses a method for administrating the adenovirus of the present invention to a subject in need of vaccination to preven an infectious disease or to treat an infectious disease, two or more times thereof which may comprise administering to the subject in need thereof a therapeutically effective amount of the adenovirus described herein wherein the non-native amino acid shields the vector from humoral responses. Other and further aspects, features, and advantages of the present invention will be apparent from the following description of the presently preferred embodiments of the invention given for the purpose of disclosure.

Other and further aspects, features, and advantages of the present invention will be apparent from the following description of the presently preferred embodiments of the invention given for the purpose of disclosure.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1A is a schematic representation of a cross section of Ad viral particle. Major capsid proteins fiber (IV), hexon (II), and penton base (III) are indicated on the left. Core proteins V, VII, and Mu are indicated on the bottom. Cement capsid proteins VI, IIIa, VIII, and IX (in red) are indicated on the right.

FIG. 1B is a schematic representation of a vector containing an unmodified adenoviral capsid.

FIG. 1C is a schematic representation of a vector containing a modified adenoviral coat and the genetic elements in accordance with the present invention.

FIG. 2 is an analysis of Ad-wt-pIX-TK DNA content and pIX-TK virion incorporation of cesium chloride (CsCl) gradient fractions. (a) DNA content of individual gradient fractions of Ad-wt-pIX-TK (where HSV-thymidine kinase (TK) is fused to pIX) was determined by measuring absorbance at 260 nm. (b) Individual fractions were analyzed for pIX-TK fusion protein using an anti-flag antibody following SDS-PAGE and transfer to PVDF membrane. Fractions 6-14 are from the lower gradient band and are of complete particles (indicated by DNA content) while fractions 23-30 are from the upper gradient band of empty particles. pIX-EGFP is indicated on the western blot. The upper bands on the western blot represent pIX-TK and are higher due to the larger size of HSV-TK in comparison to EGFP.

FIG. 3 shows a demonstration of the shielding effect of TK when fused to pIX to protect virions from recognization with neutralizing antibodies. A. Demonstration of the concept of the ELISA methodology for analysis of shielding effect. Administration of wild-type Ad5 vector evokes antibody production against capsid proteins from the Ad virion. When the sera is tested against bound virions, the antibodies can detect wild type Ad, while they are unable to detect shielded Ad vectors. B. Demonstration of the shielding effect of TK when fused to pIX to protect virions from recognization with neutralizing antibodies. Ad.pIX-TK or Ad.pIX-WT (wild type pIX) virions were coated on a 96 well plate for overnight incubation at 4 C. The following day wells were washed 4 times with PBS/Tween-20 and then blocked with 3% milk/1% PBS for greater than one hour. After washing 4 times with PBS/Tween20 serum from pre-immunized or post-immunized mice was added at 1:10 (use 3% milk/PBS to dilute) and plates incubated at room temperature for 2 hours. Wells were then washed 6 times with PBS/Tween20 and HRP conjugated Goat anti-mouse IgG (diluted 1:5000 in 3% milk/PBS) was added for 1 hour at room temperature. After washing 6 times, TMB was added, and after 10 minutes maximum the reaction was stopped with 0.4N H2SO4. Plate was read using 450 nm-650 nm. Serum for these experiments was derived from C57BL/6 mice. Mice were 3 month old C57BL/6 and were injected with 8×10⁸ viral particles of wild type Ad5 through tail vein. Blood samples were collected at different times to monitor antibody response. Post-immunized serum was taken at 8 days for this experiment.

FIG. 4 depicts the incorporation of a docking molecule into pIX allows conjugation the Ad vector with a matching ligand. After the first CsCl ultracentrifugation of cell lysate infected with Ad5.pIX-Zc, the collected viruses were incubated in vitro at room temperature with huIgG. Then a complex formed by association of IgG with Ad5.pIX-Zc was purified from unincorporated ligands on CsCl gradients and an aliquot corresponding to 5×10⁹ viral particle was analyzed by immunoblotting alongside sample of Ad vector, which was not incubated with IgG. Western blot analysis of purified viruses with anti-IgG Abs showed the presence of heavy (H) and light (L) chain of IgG in preparation of complex. Additionally, detection of modified pIX protein demonstrates capacity of Zc domain to restore its function to bind IgG after denaturation.

FIG. 5 depicts a scheme to show effects of pIX-modification on the anti-Ad antibody production in response to vaccination with Ad vectors. Control Ad vector, with wild type pIX shown in top panel, while experimental vector with modified pIX (shield protein represented by flags) shown in bottom panel. Anti-Ad production should increase in response to control Ad vector multiple administration correlating with decreased transgene expression. Anti-Ad production should be negligible or remain at low level and transgene expression should remain at a similar level.

FIG. 6 depicts a scheme of evading neutralizing antibodies by pIX-modified Ad vectors. Animals are treated as naïve (not shown) or pre-immunized with wild type Ad5. Control Ad vector, with wild type pIX shown in top panel, while experimental vector with modified pIX (shield protein represented by flags) shown in bottom panel. Wild type-pIX vectors should not escape neutralizing antibodies, and thus transgene expression should decrease. pIX-modified vectors should be shielded from neutralizing antibodies and hence transgene expression will not be affected.

FIG. 7 depicts oncolytic viral spread.

FIG. 8 depicts adenovirus entry pathway. The primary binding of the virus to CAR [36, 37] is mediated by the knob domain of the fiber protein (Henry et al. (1994) J Virol 68: 5239-5246.) followed by internalization of the virus within an endosome triggered by a secondary interaction of the RGD motif of adenovirus penton base protein with cellular integrins, αᵥβ₃ and αvβ5 (Wickham et al. (1993) Cell 73: 309-319 and Wickham et al. (1994) J Cell Biol 127: 257-264). The virus then escapes from the endosome and, after partial uncoating, translocates to the nuclear pore complex and releases its genome into the nucleoplasm where subsequent steps of viral replication take place.

FIG. 9 depicts a cytopathic effect and spread of Ad-IX-EGFP. Cytopathic effect of Ad-CMV-EGFP (—□—) and Ad-IX-EGFP (—∘—) (both E1 deleted) in 293 cells (complementary pIX expression) and 911 cells (no pIX expression) at various multiplicities of infection. Cytotoxicity measured by a non-radioactive proliferation assay and expressed as percentage of non-infected cells (n=6).

FIGS. 10A and 10B depict the plasmid map and sequence of pSILucIXNhe.

FIGS. 11A and 11B depict the plasmid map and sequence of pSILucIX-75A-NheI.

FIGS. 12A and 12B depict the plasmid map and sequence of pSILucIX-ABD-3.

FIGS. 13A and 13B depict the plasmid map and sequence of pSILucIX-ABD-AS.

FIGS. 14A and 14B depict the plasmid map and sequence of pSILucIX-PAB.

FIGS. 15A and 15B depict the plasmid map and sequence of pSILucIX-hALB.

FIGS. 16A and 16B depict the plasmid map and sequence of pSILucIX-hALBd.

FIGS. 17A and 17B depict the plasmid map and sequence of pSILucIX-ANXV.

FIG. 18 depicts the incorporation of pIX-ABD into virions.

FIGS. 19A-19C depicts the detection of human and mouse albumin by pIX-ABD-3 and pIX-ABD-AS fusion proteins.

### DETAILED DESCRIPTION

A limitation of Ad vectors is that they induce a significant humoral response when delivered to a mammal. Therefore an Ad vectors clinical utilitwould be greatly improved if it could avoid humoral responses. Vectors that include a large protein which would providing a shield against the humoral responses is the preferred embodiment of the present invention.

The present invention provides methods useful in the administration of adenoviral vectors to animals. The ability to target an adenoviral vector and to administer repeatedly a therapeutic adenoviral vector in a clinical setting is useful in improving treatment efficacy and in enabling the treatment of diseases. This invention provides a method for repeat administration of an adenoviral gene transfer vector comprising an exogenous gene or for repeat administration of a replication competent adenoviral vector to deferent tissues of an animal. This invention also provides a method to limit the infection of non-target tissue following administration of an adenoviral vector to a particular tissue of an animal. The vector targeting potential is useful for a large number of applications, particularly, solid tumors, administration, as the risk of misinjection of the adenoviral vector is high. The present invention also provides a method for adenoviral vector repeat administration systemically which is useful for the treatment of disseminated diseases, like metastases. This invention also provides a method for repeat administration of an adenoviral gene transfer vector used as a prophylactic or therapeutic vaccine for infectious diseases.

In accordance with the present invention, there may be employed conventional molecular biology, microbiology, and recombinant DNA techniques within the skill of the art. Such techniques are explained fully in the literature. See, e.g., Maniatis, Fritsch & Sambrook, “Molecular Cloning: A Laboratory Manual (1982); “DNA Cloning: A Practical Approach,” Volumes I and II (D. N. Glover ed. 1985); “Oligonucleotide Synthesis” (M. J. Gait ed. 1984); “Nucleic Acid Hybridization” [B. D. Hames & S. J. Higgins eds. (1985)]; “Transcription and Translation” [B. D. Hames & S. J. Higgins eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984). Therefore, if appearing herein, the following terms shall have the terminology set out below.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure herein according to the normal convention of giving only the sequence in the 5′ to 3′ direction along the nontranscribed strand of DNA (i.e., the strand having a sequence homologous to the mRNA).

A “vector” is a replicon, such as plasmid, phage or cosmid, to which another DNA segment may be attached so as to bring about the replication of the attached segment. A “replicon” is any genetic element (e.g., plasmid, chromosome, virus) that functions as an autonomous unit of DNA replication in vivo; i.e., capable of replication under its own control. An “origin of replication” refers to those DNA sequences that participate in DNA synthesis. An “expression control sequence” is a DNA sequence that controls and regulates the transcription and translation of another DNA sequence. A coding sequence is “operably linked” and “under the control” of transcriptional and translational control sequences in a cell when RNA polymerase transcribes the coding sequence into mRNA, which is then translated into the protein encoded by the coding sequence.

In general, expression vectors containing promoter sequences which facilitate the efficient transcription and translation of the inserted DNA fragment are used in connection with the host. The expression vector typically contains an origin of replication, promoter(s), terminator(s), as well as specific genes which are capable of providing phenotypic selection in transformed cells. The transformed hosts can be fermented and cultured according to means known in the art to achieve optimal cell growth.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence. A “cDNA” is defined as copy-DNA or complementary-DNA, and is a product of a reverse transcription reaction from an mRNA transcript.

Transcriptional and translational control sequences are DNA regulatory sequences, such as promoters, enhancers, polyadenylation signals, terminators, and the like, that provide for the expression of a coding sequence in a host cell. A “cis-element” is a nucleotide sequence, also termed a “consensus sequence” or “motif”, that interacts with other proteins which can upregulate or downregulate expression of a specific gene locus. A “signal sequence” can also be included with the coding sequence. This sequence encodes a signal peptide, N-terminal to the polypeptide, that communicates to the host cell and directs the polypeptide to the appropriate cellular location. Signal sequences can be found associated with a variety of proteins native to prokaryotes and eukaryotes.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site, as well as protein binding domains (consensus sequences) responsible for the binding of RNA polymerase. Eukaryotic promoters often, but not always, contain “TATA” boxes and “CAT” boxes. Prokaryotic promoters contain Shine-Dalgarno sequences in addition to the −10 and −35 consensus sequences.

The term “oligonucleotide” is defined as a molecule comprised of two or more deoxyribonucleotides, preferably more than three. Its exact size will depend upon many factors which, in turn, depend upon the ultimate function and use of the oligonucleotide. The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use for the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence to hybridize therewith and thereby form the template for the synthesis of the extension product.

As used herein, the terms “restriction endonucleases” and “restriction enzymes” refer to enzymes which cut double-stranded DNA at or near a specific nucleotide sequence.

“Recombinant DNA technology” refers to techniques for uniting two heterologous DNA molecules, usually as a result of in vitro ligation of DNAs from different organisms. Recombinant DNA molecules are commonly produced by experiments in genetic engineering. Synonymous terms include “gene splicing”, “molecular cloning” and “genetic engineering”. The product of these manipulations results in a “recombinant” or “recombinant molecule”.

A cell has been “transformed” or “transfected” with exogenous or heterologous DNA when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a vector or plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or ancestor by mitosis. A “cell line” is a clone of a primary cell that is capable of stable growth in vitro for many generations. An organism, such as a plant or animal, that has been transformed with exogenous DNA is termed “transgenic”.

As used herein, the term “host” is meant to include not only prokaryotes but also eukaryotes such as yeast, plant and animal cells. Prokaryotic hosts may include E. coli, S. tymphimurium, Serratia marcescens and Bacillus subtilis. Eukaryotic hosts include yeasts such as Pichia pastoris, mammalian cells and insect cells and plant cells, such as Arabidopsis thaliana and Tobaccum nicotiana.

Two DNA sequences are “substantially homologous” when at least about 75% (preferably at least about 80%, and most preferably at least about 90% or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. In another example, the coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein. For example, a polynucleotide, may be placed by genetic engineering techniques into a plasmid or vector derived from a different source, and is a heterologous polynucleotide. A promoter removed from its native coding sequence and operatively linked to a coding sequence other than the native sequence is a heterologous promoter.

In addition, the invention may include portions or fragments of the fiber or fibritin genes. As used herein, “fragment” or “portion” as applied to a gene or a polypeptide, will ordinarily be at least 10 residues, more typically at least 20 residues, and preferably at least 30 (e.g., 50) residues in length, but less than the entire, intact sequence. Fragments of these genes can be generated by methods known to those skilled in the art, e.g., by restriction digestion of naturally occurring or recombinant fiber or fibritin genes, by recombinant DNA techniques using a vector that encodes a defined fragment of the fiber or fibritin gene, or by chemical synthesis.

As used herein, “chimera” or “chimeric” refers to a single transcription unit possessing multiple components, often but not necessarily from different organisms. As used herein, “chimeric” is used to refer to tandemly arranged coding sequence (in this case, that which usually codes for the adenovirus fiber gene) that have been genetically engineered to result in a protein possessing region corresponding to the functions or activities of the individual coding sequences.

The “native biosynthesis profile” of the chimeric fiber protein as used herein is defined as exhibiting correct trimerization, proper association with the adenovirus capsid, ability of the ligand to bind its target, etc. The ability of a candidate chimeric fiber-fibritin-ligand protein fragment to exhibit the “native biosynthesis profile” can be assessed by methods described herein.

As used herein, a “self protein” is produced by a mammal and does not induce signific humoral response against that specific protein when delivered in a reasonable quantity to mammals of the same species or genus.

A standard northern blot assay can be used to ascertain the relative amounts of mRNA in a cell or tissue in accordance with conventional northern hybridization techniques known to those persons of ordinary skill in the art. Alternatively, a standard Southern blot assay may be used to confirm the presence and the copy number of the gene of interest in accordance with conventional Southern hybridization techniques known to those of ordinary skill in the art. Both the northern blot and Southern blot use a hybridization probe, e.g. radiolabelled cDNA or oligonucleotide of at least 20 (preferably at least 30, more preferably at least 50, and most preferably at least 100 consecutive nucleotides in length). The DNA hybridization probe can be labelled by any of the many different methods known to those skilled in this art.

Hybridization reactions can be performed under conditions of different “stringency.” Conditions that increase stringency of a hybridization reaction are well known. See for examples, “Molecular Cloning: A Laboratory Manual”, second edition (Sambrook et al. 1989). Examples of relevant conditions include (in order of increasing stringency): incubation temperatures of 25° C., 37° C., 50° C., and 68° C.; buffer concentrations of 10×SSC, 6×SSC, 1×SSC, 0.1×SSC (where SSC is 0.15 M NaCl and 15 mM citrate buffer) and their equivalent using other buffer systems; formamide concentrations of 0%, 25%, 50%, and 75%; incubation times from 5 minutes to 24 hours; 1, 2 or more washing steps; wash incubation times of 1, 2, or 15 minutes; and wash solutions of 6×SSC, 1×SSC, 0.1×SSC, or deionized water.

The labels most commonly employed for these studies are radioactive elements, enzymes, chemicals which fluoresce when exposed to ultraviolet light, and others. A number of fluorescent materials are known and can be utilized as labels. These include, for example, fluorescein, rhodamine, auramine, Texas Red, AMCA blue and Lucifer Yellow. A particular detecting material is anti-rabbit antibody prepared in goats and conjugated with fluorescein through an isothiocyanate. Proteins can also be labeled with a radioactive element or with an enzyme. The radioactive label can be detected by any of the currently available counting procedures. The preferred isotope may be selected from ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re.

Enzyme labels are likewise useful, and can be detected by any of the presently utilized calorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase. U.S. Pat. Nos. 3,654,090, 3,850,752, and 4,016,043 are referred to by way of example for their disclosure of alternate labeling material and methods.

As used herein, the terms “fiber gene” and “fiber” refer to the gene encoding the adenovirus fiber protein. As used herein, “chimeric fiber protein” refers to a modified fiber gene as described above.

As used herein the term “physiologic ligand” refers to a ligand for a cell surface receptor.

The term “exogenous gene,” as it is used herein, refers to any gene in an adenoviral gene transfer vector that is not native to the adenovirus that comprises the adenoviral vector. The gene includes a nucleic acid sequence encoding a gene product operably linked to a promoter. Any portion of the gene can be non-native to the adenovirus that comprises the adenoviral gene transfer vector. For example, the gene can comprise a non-native nucleic acid sequence encoding a gene product operably linked to a native promoter, or a native nucleic acid sequence encoding a gene product operably linked to a non-native promoter or in a non-native location within the adenoviral vector. It should be appreciated that the exogenous gene can be any gene encoding an RNA or protein of interest to the skilled artisan. Therapeutic genes, genes encoding a protein that is to be studied in vitro and/or in vivo, antisense nucleic acids, and modified viral genes are illustrative of possible exogenous genes.

The term “adenoviral gene transfer vector,” as it is used herein, refers to any adenoviral vector with an exogenous gene encoding a gene product inserted into its genome. The vector must be capable of replicating and being packaged when any deficient essential genes are provided in trans.

The term “replication competent adenoviral vector,” as it is used herein, refers to any adenoviral vector that is not deficient in any gene function required for viral replication in specific cells or tissues. The vector must be capable of replicating and being packaged, but might replicate only conditionally in specific cells or tissues wherein any deficient essential genes are provided in trans. An adenoviral vector desirably contains at least a portion of each terminal repeat required to support the replication of the viral DNA, preferably at least about 90% of the full ITR sequence, and the DNA required to encapsidate the genome into a viral capsid. Many suitable adenoviral vectors have been described in the art.

The adenoviral gene transfer vector is preferably deficient in at least one gene function required for viral replication. Preferably, the adenoviral gene transfer vector is deficient in at least one essential gene function of the E1 region of the adenoviral genome, particularly the E1a region, more preferably, the vector is deficient in at least one essential gene function of the E1 region and part of the E3 region (e.g., an Xba I deletion of the E3 region) or, alternatively, the vector is deficient in at least one essential gene function of the E1 region and at least one essential gene function of the E4 region. However, adenoviral gene transfer vectors deficient in at least one essential gene function of the E2a region and adenoviral gene transfer vectors deficient in the E3 region also are contemplated here and are well-known in the art. Suitable replication-deficient adenoviral gene transfer vectors are disclosed in International Patent Applications WO 95/34671 and WO 97/21826. For example, suitable replication-deficient adenoviral gene transfer vectors include those with a partial deletion of the E1a region, a partial deletion of the E1b region, a partial deletion of the E2a region, and a partial deletion of the E3 region. Alternatively, the replication-deficient adenoviral gene transfer vector can have a deletion of the E1 region, a partial deletion of the E3 region, and a partial deletion of the E4 region.

The exogenous gene can be inserted into any suitable region of the adenoviral gene transfer vector as an expression cassette. Preferably, the DNA segment is inserted into the E1 region of the adenoviral gene transfer vector. Whereas the DNA segment can be inserted as an expression cassette in any suitable orientation in any suitable region of the adenoviral gene transfer vector, preferably, the orientation of the DNA segment is from right to left. By the expression cassette having an orientation from right to left, it is meant that the direction of transcription of the expression cassette is opposite that of the region of the adenoviral gene transfer vector into which the expression cassette is inserted.

Alternatively, the adenoviral vector is preferably conditionally replication deficient in at least one gene function required for viral replication in specific cells or tissues. Preferably, the adenoviral vector is deleted in at least one essential gene of the E1 region of the adenoviral genome, particularly the E1a region, more preferably, the vector is deficient in the retinoblastoma (Rb) binding site as described in U.S. Pat. No. 6,824,771.

It should be appreciated that the deletion of different regions of the adenoviral gene transfer vector can alter the immune response of the mammal, in particular, deletion of different regions can reduce the inflammatory response generated by the adenoviral gene transfer vector. Furthermore, the adenoviral gene transfer vector's coat protein can be modified so as to decrease the adenoviral gene transfer vector's ability or inability to be recognized by a neutralizing antibody directed against the wild-type coat protein, as described in International Patent Application WO 98/40509. Other suitable modifications to the adenoviral gene transfer vector are described in U.S. Pat. Nos. 5,559,099; 5,731,190; 5,712,136; and 5,846,782 and International Patent Applications WO 97/20051, WO 98/07877, and WO 98/54346.

Host immune evasion has been realized by the use of chemical conjugation of bi-functional polymers, such as polyethylene glycol (PEG), to the Ad capsid as described in U.S. Pat. Nos. 6,399,385. The possibility of coating Ad with polymers was also described in International Patent Application WO 00/74722. However, this methodology eliminates targeting the vector by genetically incorporated sequences. Furthermore, this method could not be used for replicating vectors as once the virus replicates, the virus progeny loses its coating and is subject to the same immunological constraints as a non-coated vector.

In one embodiment, the invention encompasses a self-assembly shielding approach wherein self proteins are directly incorporated into adenoviral vectors. The present invention pertains to viral or non-viral vectors, but preferably replication deficient or a replication competent adenoviral vectors as described herein. These vectors comprising moieties covering and shielding the vector from the effects of humoral immune responses by methods described below. Proteins such as, but not limited to, albumin, antibody fragments such as scFv or other alternate self proteins (from the serum or cytosol of cells), may be incorporated into the adenoviral capsid. Examples of such proteins may include, but are not limited to, albumin, complement regulatory factors, soluble forms of fibronectin and fibrinogen, and the proinflammatory molecules plasmin(ogen) and kininogen.

Alternate shield proteins also include, but are not limited to, ligands smaller than HSV-TK and ligands larger than HSV-TK. Ligands smaller than HSV-TK that may be used as shield proteins include, but are not limited to, α-crystallin domain and small heat shock proteins, α-1-microglobulin, β-2-microglobulin and myoglobin. Ligands of similar size or larger than HSV-TK that may be used as shield proteins include, but are not limited to, alpha-1-antitrypsin, annexins (e.g., annexin 1, annexin 2 and annexin 5) and HSP70.

Other proteins which may be useful for shielding include, but are not limited to, thyroxine-binding prealbumin, retinol-binding protein, albumin, galactoglycoprotein, α-globulins (e.g., α1-acid glycoprotein, α1-antitrypsin, α1-fetoprotein, 9.5 S α1-glycoprotein (serum amyloid P protein), GC globulin, ceruloplasmin, 3.8 S histidine-rich α2-glycoprotein, α2-macroglobulin, 4 S α2, β1-glycoprotein, α1B-glycoprotein, α1T-glycoprotein, α1-antichymotrypsin, α1-microglobulin, Zn-α2-glycoprotein, α2HS-glycoprotein, pregnancy-associated α2-glycoprotein, 3.1 S leucine-rich α2-glycoprotein, 8 S α3-glycoprotein, serum cholinesterase, thyroxine-binding globulin, inter-α-trypsin inhibitor, transcortin, haptoglobin (such as type 1-1, type 2-1, type 2-2), 13-globulins (e.g., hemopexin, transferrin, β2-microglobulin, β2-glycoprotein I, β2-glycoprotein II (C3 proactivator), β2-glycoprotein III, C-reactive protein, fibronectin), low-molecular weight proteins (e.g., lysozyme, basic protein B1, basic protein B2, 0.6 S γ2-globulin, 2 S γ2-globulin, post γ-globulin), complement components (e.g., C1q component, C1r component, C1s component, C2 component, C3 component, C4 component, C5 component, C6 component, C7 component, C8 component, C9 component), other complement factors (e.g., C1 esterase inhibitor, factor B, factor D, factor H, C4 binding protein, properdin), coagulation proteins (e.g., antithrombin III, prothrombin, antihemophilic factor (Factor VIII), plasminogen, fibrin-stabilizing factor (Factor XIII), fibrinogen) and immunoglobulins (e.g., immunoglobulin G, immunoglobulin A, immunoglobulin M, immunoglobulin D, immunoglobulin E, κ Bence Jones protein, γ Bence Jones protein).

In another embodiment, the incorporation of a molecule into the C terminus of pIX, which can then conjugate to a protein, could accomplish a shielding approach. Biotin acceptor peptide is one such molecule that has already been used to allow non-covalent attachment of functional moieties in a pharmacological manner at the pIX locus (Campos et al. 2004, Mol. Ther. 9:942-954). Proteins which bind human extracellular proteins, dominating human plasma proteins such as albumin or immunoglobulins, or any fragment thereof with high affinity and specificity may also be considered for methods of the present invention. Many bacteria express such proteins on their surface, including but not limited to protein A of Staphylococcus aureas, protein G of group C and G streptococci, protein L, M proteins of streptococcal Fc receptors, MSCRAMMs (microbial surface components recognizing adhesive matrix molecules) and protein PAB from Peptostreptococcus magnus (see, e.g., Navarre & Schneewind, Microbiology and Molecular Biology Reviews, March 1999, p. 174-229, Vol. 63, No. 1). In another embodiment, mutant proteins may also be used in the present invention. Examples of mutants include, but are not limited to, mutant versions of Fc-binding domain of Staphylococcus aureus protein A (see, e.g., Korokhov et al. J. Virol. 2003 December; 77(24):12931-40 and U.S. patent application Ser. No. 10/859,739, the disclosures of which are incorporated by reference). These proteins or peptides may be incorporated into the fiber (e.g. C-terminus, HI loop etc.), hexon (e.g. loop 1, loop 2, etc.), penton base (e.g. close to or replacing the RGD domain etc.), pIX (e.g. C-terminal), pIII (e.g. N-terminal) or any combination thereof. These domains are of a size compatible with pIX incorporation and would permit the conjugation to Fc-fusion proteins or humanized antibodies thus providing the necessary shielding effect with additional targeting functions provided.

Once the described vectors are constructed, they may be incubated in vitro with shielding moieties, such as, but not limited to, human serum albumin (HSA) or antibodies. The vectors may be injected also directly in vivo to an animal or preferably a human without pre-incubation. The vector in this case may be coated with self proteins of the individual animal or person avoiding complications of transferring a foreign substance. The coated vector may have a longer circulation time in the body, providing sufficient time to reach its target (e.g. metastasis or cancer cell, target organ etc.) Furthermore, the coated vector may stay in the circulatory system longer than the uncoated vector even in the presence of antibodies against the viral coat proteins. It is also possible to multiply dose such vectors for improved efficacy.

Adenoviral gene transfer vectors can be specifically targeted through a chimeric adenovirus coat protein comprising a normative amino acid sequence, wherein the chimeric adenovirus coat protein directs entry into a specific cell of an adenoviral gene transfer vector comprising the chimeric adenovirus coat protein that is more efficient than entry into a specific cell of an adenoviral gene transfer vector that is identical except for comprising a wild-type adenovirus coat protein rather than the chimeric adenovirus coat protein. The chimeric adenovirus coat protein comprising a normative amino acid sequence can serve to increase efficiency by decreasing non-target cell transduction by the adenoviral gene transfer vector.

The normative amino acid sequence of the chimeric adenovirus coat protein, which comprises from about 3 amino acids to about 30 amino acids, can be inserted into or in place of an internal coat protein sequence, or, alternatively, the normative amino acid sequence can be at or near the C-terminus of the chimeric adenovirus coat protein. The chimeric adenovirus coat protein can be a fiber protein, a penton base protein, a hexon or a pIX protein. In addition, the normative amino acid sequence can be linked to the chimeric adenovirus coat protein by a spacer sequence of from about 3 amino acids to about 30 amino acids. Targeting through a chimeric adenovirus coat protein is described generally in U.S. Pat. Nos. 5,559,099; 5,712,136; 5,731,190; 5,770,440; 5,871,726; and 5,830,686 and International Patent Applications WO 96/07734, WO 98/07877, WO 97/07865, WO 98/54346, WO 96/26281, and WO 98/40509. An adenoviral gene transfer vector that comprises a chimeric coat protein comprising a normative amino acid sequence in accordance with U.S. Pat. No. 5,965,541 or WO 97/20051, such as one that comprises polylysine as the normative amino acid sequence, can be used to re-administer an exogenous gene encoding a gene product to a particular muscle of an animal. The use of such a vector to repeat administration can result in a higher level of expression of the gene product as compared to an adenoviral vector in which the corresponding adenoviral coat protein has not been modified to comprise a normative amino acid sequence, such as polylysine.

The chimeric adenovirus coat protein can be a pIX protein. Targeting through a chimeric adenovirus pIX coat protein is described generally in U.S. Pat. Nos. 6,740,525 and 6,555,368. The present invention provides a chimeric protein IX. The pIX gene may be modified by inserting a DNA sequence encoding a single chain antibody into the 3′ end of the pIX gene, resulting in a stabilized antibody inserted at the C terminus of the pIX protein. The chimeric pIX protein has an adenoviral pIX domain and also a non-native amino acid, where the non-native amino acid shields the adenovirus from humoral immune responses. In one embodiment, the present invention provides a shielding moiety that is a ligand that binds to a substrate present on the surface cells. In this case, the chimeric pIX can be used to target vectors containing such proteins to desired cell types. Thus, the invention provides vector systems including such chimeric pIX proteins that shield from neutralizing antibodies as well as methods of infecting cells using such vector systems.

In other embodiments of the invention, the chimeric protein may be a chimeric pIIIa. The minor capsid protein pIIIa gene may be modified by inserting a DNA sequence encoding a stabilized antibody into the 5′ end of the pIIIa gene, resulting in a stabilized antibody inserted at the N terminus of the pIII protein. In another embodiment, the chimeric adenoviral proteins are derived from a fiber, a penton, a hexon protein or a protein VI.

The non-native amino acid sequence can, but need not be a discrete domain or stretch of contiguous amino acids. In other words, the non-native amino acid sequence can be generated by the particular confirmation of the protein, e.g., through folding of the protein in such a way as to bring contiguous and/or noncontiguous sequences into mutual proximity. Thus, for example, the non-native amino acid can be constrained by a peptide loop within the chimeric protein (formed, for example, by a disulfide bond between non-adjacent amino acids of said protein). Typically, the protein is a fusion protein in which the non-native amino acid sequence is a discrete domain of the protein fused to the pIX domain. Preferably, in this configuration, a non-native amino acid sequence can constitute the C-terminus of the protein. The non-native amino acid sequence can be any desired amino acid sequence, so long as it is not native to a wild-type adenoviral pIX protein and shields the adenovirus from humoral responses.

In many embodiments, the non-native amino acid sequence is a ligand (i.e., a domain that binds a discrete substrate or class of substrates). However, the non-native amino acid sequence can be other classes of polypeptides (e.g., an antibody or a derivative thereof, such as a single chain antibody (scFv) or Fab (i.e., a univalent antibody or a fragment of an immunoglobulin consisting of one light chain linked through a disulphide bond to a portion of the heavy chain, containing one antigen binding site), an antigen, an epitope, a glycosylation or phosphorylation signal, a protease recognition sequence, etc.), if desired.

In a preferred embodiment, the non-native amino acid is an antibody, advantageously a single chain antibody, more advantageously a stabilized single chain antibody. The stabilized antibody of the present invention encompasses all stabilized antibodies known or developed by one of skill in the art. In a preferred embodiment, the stabilized antibody may be a single chain antibody (scFv), such as a humanized scFv (see, e.g., Graff et al. in Protein Eng Des Sel. 2004 April; 17(4):293-304). The stabilized antibodies of the present invention also encompass disulfide stabilized antibodies, wherein the heavy and light chains of the antibody are associated by disulfide bonds rather than a peptide linker (see, e.g., U.S. Pat. Nos. 6,639,057 and 6,538,111). In other preferred embodiments, the stabilized antibody may be a mini antibody or a heavy chain variable domain (dAb) (see, e.g., Jespers et al. in Nat. Biotechnol. 2004 September; 22(9):1161-5). In yet another embodiment, the stabilized antibody may be a polymer conjugates which exhibits stabilized antibody binding capacity (see, e.g., U.S. Pat. Nos. 6,538,104 and 6,491,923). The invention also encompasses stabilized antibodies produced by the method of U.S. Pat. No. 6,262,238 wherein stabilized antibodies free of disulfide bridges are obtained by substituting the cysteines which form disulfide bridges by other amino acids and replacing at least one, and preferably two or more amino acids by stability-mediating amino acids. The invention also encompasses the stabilized, divalent antigen-binding antibody fragments of U.S. Pat. No. 5,506,342. The only requirement for the stabilized antibodies of the present invention is the ability of the stabilized antibody to accomplish cytosol-to-nuclear transport and nuclear residence as an Ad capsid component, while retaining its key conformational aspects dictating antigen recognition and binding.

In another embodiment of the invention, the stabilized single chain antibody (scFV) comprises mutations in the scFv CDR regions. Any mutations, which preserve an ability of scFv in the context of Ad capsid to bind an antigen are suitable for methods of the invention. Examples of scFv stabilizing mutations include, but are not limited to, those mutations described in Arndt et al., J Mol Biol 2001 Sep. 7; 312(1):221-8; Bestagno et al., Biochemistry 2001 Sep. 4; 40(35):10686-92 and Rajpal et al., Proteins 2000 Jul. 1; 40(1):49-57, the disclosures of which are incorporated by reference. A stabilized scFv “framework” is developed via directed mutations in the scFv CDR regions. These stabilized CDRs' framework can then serve as a scaffold onto which scFv variable domains, which embody antigen recognition, can then be grafted by molecular engineering methods. The chimeric scFv thus manifests the desired antigen recognition profile while also embodying the stability of the scaffold CDR domain.

In a preferred embodiment, the stabilized antibody is targeted to a cell surface marker of a tumor cell. Cell surface markers that can be targeted according to the methods of the present invention include, but are not limited to, CD40, DC-SIGN, DEC-205, CEA and PSMA. In one embodiment, the stabilized scFv ligand is an anti-CD40 scFv.

Although, the non-native amino acid sequence could be of any origin, in the preferred embodiment of the invention, it is immunologically tolerated by the species to which it is delivered. For example, if the albumin sequence is used as non-native amino acid sequence, it is preferable, that the human sequence is used for human clinical use.

The present invention also relates to adenoviral capsids, preferably an adenoviral capsid which may comprise any one or more of the above-described chimeric proteins. In one embodiment, the adenoviral capsid may bind dendritic cells. In another embodiment, the adenoviral capsid may comprise a mutant adenoviral cellular receptor, wherein the mutant adenoviral cellular receptor may have an affinity for a native adenoviral cellular receptor of at least about an order of magnitude less than a wild-type adenoviral fiber protein. The adenoviral capsid may comprise an adenoviral penton base protein having a mutation affecting at least one native RGD sequence and/or at least one native HVR sequence. In another embodiment, the adenoviral capsid may lack a native glycosylation or phosphorylation site. In yet another embodiment, the adenoviral capsid may elicit less immunogenicity in a host animal as compared to a wild-type adenovirus. In another embodiment, the adenoviral capsid may comprise a second non-adenoviral ligand advantageously conjugated to a fiber, a penton, a hexon, a protein IIIa or a protein VI. In yet another embodiment, the non-native amino acid of the adenoviral capsid may comprise a ligand and a second non-adenoviral ligand recognizes the same substrate as the non-native amino acid.

Methods for making and/or administering a vector or recombinants or plasmid for expression of gene products of genes of the invention either in vivo or in vitro can be any desired method, e.g., a method which is by or analogous to the methods disclosed in, or disclosed in documents cited in: U.S. Pat. Nos. 4,603,112; 4,769,330; 4,394,448; 4,722,848; 4,745,051; 4,769,331; 4,945,050; 5,494,807; 5,514,375; 5,744,140; 5,744,141; 5,756,103; 5,762,938; 5,766,599; 5,990,091; 5,174,993; 5,505,941; 5,338,683; 5,494,807; 5,591,639; 5,589,466; 5,677,178; 5,591,439; 5,552,143; 5,580,859; 6,130,066; 6,004,777; 6,130,066; 6,497,883; 6,464,984; 6,451,770; 6,391,314; 6,387,376; 6,376,473; 6,368,603; 6,348,196; 6,306,400; 6,228,846; 6,221,362; 6,217,883; 6,207,166; 6,207,165; 6,159,477; 6,153,199; 6,090,393; 6,074,649; 6,045,803; 6,033,670; 6,485,729; 6,103,526; 6,224,882; 6,312,682; 6,348,450 and 6; 312,683; U.S. patent application Ser. No. 920,197, filed Oct. 16, 1986; WO 90/01543; WO91/11525; WO 94/16716; WO 96/39491; WO 98/33510; EP 265785; EP 0 370 573; Andreansky et al., Proc. Natl. Acad. Sci. USA 1996; 93:11313-11318; Ballay et al., EMBO J. 1993; 4:3861-65; Felgner et al., J. Biol. Chem. 1994; 269:2550-2561; Frolov et al., Proc. Natl. Acad. Sci. USA 1996; 93:11371-11377; Graham, Tibtech 1990; 8:85-87; Grunhaus et al., Sem. Virol. 1992; 3:237-52; Ju et al., Diabetologia 1998; 41:736-739; Kitson et al., J. Virol. 1991; 65:3068-3075; McClements et al., Proc. Natl. Acad. Sci. USA 1996; 93:11414-11420; Moss, Proc. Natl. Acad. Sci. USA 1996; 93:11341-11348; Paoletti, Proc. Natl. Acad. Sci. USA 1996; 93:11349-11353; Pennock et al., Mol. Cell. Biol. 1984; 4:399-406; Richardson (Ed), Methods in Molecular Biology 1995; 39, “Baculovirus Expression Protocols,” Humana Press Inc.; Smith et al. (1983) Mol. Cell. Biol. 1983; 3:2156-2165; Robertson et al., Proc. Natl. Acad. Sci. USA 1996; 93:11334-11340; Robinson et al., Sem. Immunol. 1997; 9:271; and Roizman, Proc. Natl. Acad. Sci. USA 1996; 93:11307-11312.

According to one embodiment of the invention, the expression vector is a viral vector, in particular an in vivo expression vector. In an advantageous embodiment, the expression vector is an adenovirus vector, such as a human adenovirus (HAV) or a canine adenovirus (CAV). Advantageously, the adenovirus is a human Ad5 vector, an E1-deleted adenovirus or an E3-deleted adenovirus.

In one embodiment the viral vector is a human adenovirus, in particular a serotype 5 adenovirus, rendered incompetent for replication by a deletion in the E1 region of the viral genome. The deleted adenovirus is propagated in E1-expressing 293 cells or PER cells, in particular PER.C6 (F. Falloux et al Human Gene Therapy 1998, 9, 1909-1917). The human adenovirus can be deleted in the E3 region eventually in combination with a deletion in the E1 region (see, e.g. J. Shriver et al. Nature, 2002, 415, 331-335, F. Graham et al Methods in Molecular Biology Vol. 7: Gene Transfer and Expression Protocols Edited by E. Murray, The Human Press Inc, 1991, p 109-128; Y. Ilan et al Proc. Natl. Acad. Sci. 1997, 94, 2587-2592; S. Tripathy et al Proc. Natl. Acad. Sci. 1994, 91, 11557-11561; B. Tapnell Adv. Drug Deliv. Rev. 1993, 12, 185-199; X. Danthinne et al Gene Thrapy 2000, 7, 1707-1714; K. Berkner Bio Techniques 1988, 6, 616-629; K. Berkner et al Nucl. Acid Res. 1983, 11, 6003-6020; C. Chavier et al J. Virol. 1996, 70, 4805-4810). The insertion sites can be the E1 and/or E3 loci eventually after a partial or complete deletion of the E1 and/or E3 regions. Advantageously, when the expression vector is an adenovirus, the polynucleotide to be expressed is inserted under the control of a promoter functional in eukaryotic cells, such as a strong promoter, preferably a cytomegalovirus immediate-early gene promoter (CMV-IE promoter). The CMV-IE promoter is advantageously of murine or human origin. The promoter of the elongation factor 1α can also be used. In one particular embodiment a promoter regulated by hypoxia, e.g. the promoter HRE described in K. Boast et al Human Gene Therapy 1999, 13, 2197-2208), can be used. A muscle specific promoter can also be used (X. Li et al Nat. Biotechnol. 1999, 17, 241-245). Strong promoters are also discussed herein in relation to plasmid vectors. A poly(A) sequence and terminator sequence can be inserted downstream the polynucleotide to be expressed, e.g. a bovine growth hormone gene or a rabbit β-globin gene polyadenylation signal.

In another embodiment the viral vector is a canine adenovirus, in particular a CAV-2 (see, e.g. L. Fischer et al. Vaccine, 2002, 20, 3485-3497; U.S. Pat. No. 5,529,780; U.S. Pat. No. 5,688,920; PCT Application No. WO95/14102). For CAV, the insertion sites can be in the E3 region and/or in the region located between the E4 region and the right ITR region (see U.S. Pat. No. 6,090,393; U.S. Pat. No. 6,156,567). In one embodiment the insert is under the control of a promoter, such as a cytomegalovirus immediate-early gene promoter (CMV-IE promoter) or a promoter already described for a human adenovirus vector. A poly(A) sequence and terminator sequence can be inserted downstream the polynucleotide to be expressed, e.g. a bovine growth hormone gene or a rabbit β-globin gene polyadenylation signal.

The invention also provides for transformed host cells comprising such vectors. In one embodiment, the vector is introduced into the cell by transfection, electroporation or infection. The invention also provides for a method for preparing a transformed cell expressing the adenovirus of the present invention comprising transfecting, electroporating or infecting a cell with the adenovirus to produce an infected producing cell and maintaining the host cell under biological conditions sufficient for expression of the adenovirus in the host cell.

According to another embodiment of the invention, the expression vectors are expression vectors used for the in vitro expression of proteins in an appropriate cell system. The expressed proteins can be harvested in or from the culture supernatant after, or not after secretion (if there is no secretion a cell lysis typically occurs or is performed), optionally concentrated by concentration methods such as ultrafiltration and/or purified by purification means, such as affinity, ion exchange or gel filtration-type chromatography methods.

It is understood to one of skill in the art that conditions for culturing a host cell varies according to the particular gene and that routine experimentation is necessary at times to determine the optimal conditions for culturing the vector depending on the host cell. A “host cell” denotes a prokaryotic or eukaryotic cell that has been genetically altered, or is capable of being genetically altered by administration of an exogenous polynucleotide, such as a recombinant plasmid or vector. When referring to genetically altered cells, the term refers both to the originally altered cell and to the progeny thereof.

Polynucleotides comprising a desired sequence can be inserted into a suitable cloning or expression vector, and the vector in turn can be introduced into a suitable host cell for replication and amplification. Polynucleotides can be introduced into host cells by any means known in the art. The vectors containing the polynucleotides of interest can be introduced into the host cell by any of a number of appropriate means, including direct uptake, endocytosis, transfection, f-mating, electroporation, transfection employing calcium chloride, rubidium chloride, calcium phosphate, DEAE-dextran, or other substances; microprojectile bombardment; lipofection; and infection (where the vector is infectious, for instance, a retroviral vector). The choice of introducing vectors or polynucleotides will often depend on features of the host cell.

In view of the above, the method can further comprise subsequently repeating the administration of an adenoviral gene transfer vector comprising the exogenous gene encoding the gene product and/or a replication competent Ad vector with or without vector comprising the exogenous gene encoding the gene product to the appropriate tissue of the animal. All administrations are performed with Ad vectors comprising a chimera of the present invention, advantageously a chimeric pIX coat protein that protects the vector from neutralizing antibodies. Preferably further the pIX chimeric adenoviral coat protein comprising a normative amino acid sequence, wherein the chimeric adenoviral coat protein directs entry of the vector into cells more efficiently than a vector that is otherwise identical, except for comprising a corresponding wild-type adenoviral coat protein (see, e.g., U.S. Pat. No. 5,965,541, PCT Publication No. WO 97/20051 or U.S. Pat. No. 6,555,368).

Thus, the inventive virions can be targeted to cells within any organ or system, including, for example, respiratory system (e.g., trachea, upper airways, lower airways, alveoli), nervous system and sensory organs (e.g., skin, ear, nasal, tongue, eye), digestive system (e.g., oral epithelium and sensory organs, salivary glands, stomach, small intestines/duodenum, colon, gall bladder, pancreas, rectum), muscular system (e.g., skeletal muscle, connective tissue, tendons), skeletal system (e.g., joints (synovial cells), osteoclasts, osteoblasts, etc.), immune system (e.g., bone marrow, stem cells, spleen, thymus, lymphatic system, etc.), circulatory system (e.g., muscles, connective tissue, and/or endothelia of the arteries, veins, capillaries, etc.), reproductive system (e.g., testes, prostate, uterus, ovaries), urinary system (e.g., bladder, kidney, urethra), endocrine or exocrine glands (e.g., breasts, adrenal glands, pituitary glands), etc or delivered systemically. These adenoviral vectors are capable of delivering gene products with high efficiency and specificity to cells expressing receptors which recognize the ligand component of the fiber-fibritin-ligand chimera. A person having ordinary skill in this art would recognize that one may exploit a wide variety of genes encoding e.g. receptor ligands or antibody fragments which specifically recognize cell surface proteins unique to a particular cell type to be targeted.

The invention further encompasses a method for administrating the adenovirus of the present invention to a subject in need thereof which may comprise administering to the subject in need thereof a therapeutically effective amount of the adenovirus described herein wherein the non-native amino acid targets the tumor cell such that the adenovirus infects the target cells.

The present invention can be practiced with any suitable animal, preferably the present invention is practiced with a mammal, more preferably, a human. Additionally, the adenoviral vector can be a gene transfer vector or a replication competent vector and can be administered, e.g., once, twice, or more, to any suitable tissue or delivered systemically to the animal. Systemic administration can be accomplished through intravenous injection, either bolus or continuous, or any other suitable method.

After subsequent administration of the adenoviral gene transfer vector comprising an exogenous gene, production of the gene product in the tissue of the animal is desirably at least 1% of (such as at least 10% of, preferably at least 50% of, more preferably at least 80% of, and most preferably, the same as or substantially the same as) production of the gene product after initial administration of the same adenoviral gene transfer vector containing the exogenous gene. Methods for comparing the amount of gene product produced in the tissue of administration are known in the art. The comparison can be made at the same time after the initial and subsequent administrations of the adenoviral gene transfer vector.

After subsequent administration of a replication competent adenoviral vector, replication of the vector in the tissue of the animal is desirably at least 1% of (such as at least 10% of, preferably at least 50% of, more preferably at least 80% of, and most preferably, the same as or substantially the same as) replication of the vector after initial administration. Methods for comparing the amount of adenovirus replication in the tissue of administration are known in the art. The comparison can be made at the same time after the initial and subsequent administrations of the adenoviral vector.

To facilitate the administration of adenoviral vectors, they can be formulated into suitable pharmaceutical compositions. Generally, such compositions include the active ingredient (i.e., the adenoviral vector) and a pharmacologically acceptable carrier. Such compositions can be suitable for delivery of the active ingredient to a patient for medical application, and can be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention can be formulated in a conventional manner using one or more pharmacologically or physiologically acceptable carriers comprising excipients, as well as optional auxiliaries, which facilitate processing of the active compounds into preparations, which can be used pharmaceutically. Proper formulation is dependent upon the route of administration chosen. Thus, for injection, the active ingredient can be formulated in aqueous solutions, preferably in physiologically compatible buffers. For transmucosal administration, penetrants appropriate to the barrier to be permeated are used in the formulation. Such penetrants are generally known in the art. For oral administration, the active ingredient can be combined with carriers suitable for inclusion into tablets, pills, dragees, capsules, liquids, gels, syrups, slurries, suspensions and the like. For administration by inhalation, the active ingredient is conveniently delivered in the form of an aerosol spray presentation from pressurized packs or a nebuliser, with the use of a suitable propellant. The active ingredient can be formulated for parenteral administration by injection, e.g., by bolus injection or continuous infusion. Such compositions can take such forms as suspensions, solutions or emulsions in oily or aqueous vehicles, and can contain formulatory agents such as suspending, stabilizing and/or dispersing agents. Other pharmacological excipients are known in the art.

Those of ordinary skill in the art can easily make a determination of the proper dosage of the adenoviral gene transfer vector. Generally, certain factors will impact the dosage that is administered; although the proper dosage is such that, in one context, the exogenous gene is expressed and the gene product is produced in the particular muscle of the mammal. Preferably, the dosage is sufficient to have a therapeutic and/or prophylactic effect on the animal. The dosage also will vary depending upon the exogenous gene to be administered. Specifically, the dosage will vary depending upon the particular muscle of administration, including the specific adenoviral vector, exogenous gene and/or promoter utilized. For purposes of considering the dose in terms of particle units (pu), also referred to as viral particles (vp), it can be assumed that there are approximately 10-100 particles per particle forming unit (pfu) (e.g., 1×10¹⁰ pfu is equivalent to 1×10¹¹ to 1×10¹² pu).

The invention will now be further described by way of the following non-limiting examples.

### EXAMPLES

### Example 1

This example demonstrates the antibody evasion of an adenovirus incorporating GFP into the coat protein of pIX in vitro.

Genetic incorporation of eGFP into the coat protein of pIX and virus propagation. The cDNA of enhanced green fluorescent protein (eGFP) was inserted accordingly to Le et al. (2004, Mol. Imaging. 3:105-116) in frame at a NheI restriction site after a FLAG tag amino acid sequence linked to the carboxy terminus of pIX in the shuttle vector pShlpIXNhe. The plasmid was linearized with PmeI digestion to allow homologous recombination with the adenovirus genome in E. coli using standard methodologies with the commercially available AdEasy (Q-BIOgene) system. Viruses, which contain the wild type Ad5 fiber, were propagated in 911 cells, and purified by double cesium chloride ultracentrifugation as standard, then dialyzed against phosphate-buffered saline with Mg²⁺, Ca²⁺, and 10% glycerol.

In vitro antibody evasion assessment of an adenovirus incorporating GFP into the pIX coat protein. Ad-pIX-eGFP vector is pre-incubated in the presence of neutralizing antibodies, for example, rabbit anti-Ad2 polyclonal antibody (an antibody titer of 5000:1 antibody molecules to viral particles) for 1 hour, and then transduce CAR positive cells, e.g. A549, at a multiplicity of infection ((MOI) ranging from 1-100 pu per cell) for 30 minutes, at 37° C. Ability of the vector to evade neutralizing antibodies is assessed by the level of transduction of the Ad vector. The vector can be visualized using microscopy techniques in cells due to eGFP (Le et al. 2004, Molecular Imaging 3:105-116) and hence transduced cells will fluorescence. A more thorough test of the ability of the vector to evade neutralizing antibodies is examined by pre-incubating the vector with commercially available human serum. Serial dilutions of human serum are used in the pre-incubation step, and again CAR positive cells are transduced as already described with cells examined for fluorescence 24-72 hours later under the appropriate microscopy conditions. Additionally GFP levels can be examined through simple flow cytometry analysis, whereby the cells are harvested, washed in PBS and fixed, then analyzed for fluorescence on a FACScalibur (Becton-Dickinson) machine.

### Example 2

This example demonstrates the antibody evasion of an adenovirus incorporating GFP into the coat protein of pIX in vivo in GFP transgenic mice.

GFP transgenic mice are used so that antibodies against the shielding protein, GFP, will not be raised. The adenovirus incorporating GFP into pIX, as described in Example 1, is tested for the evasion of the immune system in both naïve and immunized mice. Naïve mice are not pre-exposed to a non-replicative Ad5 vector, while immunized mice are injected intravenously with 1×10¹⁰ pu of a non-replicative Ad5 vector to generate neutralizing adenovirus antibodies. Ad-pIX-GFP is administered to both groups of animals at a dose of 1×10⁹, 1×10¹⁰, 1×10¹¹ pu. Serum samples are taken at various times post-infection and adenovirus neutralizing antibodies are measured. The peak of an antibody response is expected to be detectable between 14 and 21 days following the immunization procedure Anti-adenovirus antibody profiles of the animals are observed by testing serum to block the transduction of unmodified adenovirus into A549 cells, or HeLa cells.

### Example 3

This example demonstrates the targeting activity of an adenovirus incorporating an antibody-related fragment into the coat protein of pIX in vitro.

Generation of an adenovirus containing an antibody-related fragment incorporated in the pIX coat protein. pSILucIXNhe shuttle vector, a modified form of pShlpIXNhe containing the luciferease gene, was used to insert the cDNA of a single chain antibody (scFv) at the C terminus of pIX following the FLAG tag preceding the NheI cloning site. PCR procedures created Nhe1 ends on the scFv cDNA and the resulting fragment was ligated into the Nhe1 site in this vector. The plasmid was linearized with PmeI digestion to allow homologous recombination with the Ad genome in E. coli using standard methodologies with the commercially available AdEasy system. Viruses, which contain the wild type Ad5 fiber, were propagated in 293 cells, and purified by double cesium chloride ultracentrifugation as standard, then dialyzed against 10 mM Tris buffer, with Mg²⁺, Na²⁺ and 10% glycerol.

In a further embodiment of this example, a single domain antibody, or other antibody-related fragment such as an artificial antibody mimic, maybe inserted into the pIX C terminus to achieve the same effect as that of a single-chain antibody fragment.

Targeting activity of an adenovirus incorporating an antibody-related fragment into the coat protein of pIX. The generated pIX-scFv adenovirus is used to transduce cells that are CAR negative yet express the epitope that the scFv recognizes naturally, or have been stably transfected to express the epitope in an artificial receptor system displayed using pDisplay (Invitrogen). The cells are preferably CAR negative as this version of the adenovirus still retains the wild type fiber. Either adenovirus is pre-blocked with recombinant protein that binds to the scFv (at 2 μg per 10⁹ pu), or cells are pre-blocked with antibody (20 μg per well, cells are confluent at 5×10⁵ cells per 24 well), and then cells are transduced accordingly at a range of MOI (1-1000 pu per cell) for 30 minutes on ice. This vector carries the luciferase gene, and thereby 24 hours following transduction, luciferase activity can be assessed using a commercially available kit.

### Example 4

This example demonstrates the antibody evasion of an adenovirus incorporating an antibody-related fragment into the coat protein of pIX in vitro.

In vitro antibody evasion assessment of an Ad vector incorporating antibody-related fragment into the pIX coat protein. Ad-pIX-scFv vector is pre-incubated in the presence of neutralizing Ad antibodies, or human serum as previously described in Example 1. The Ad vector, as it still contains wild type Ad5 fiber, will then be used to transduce either CAR positive cells, e.g. A549, or cell lines, which are CAR negative but express at the cell surface the target of the scFv (either naturally or in an artificial receptor system). Ability of the vector to evade neutralizing antibodies are assessed by the level of transduction of the Ad vector. As previously mentioned in Example 3, this vector carries the luciferase gene, and thereby 24 hours following transduction, luciferase activity can be assessed using a commercially available kit. In a further embodiment of this vector, the fiber is modified so that the knob is removed, yet remains trimerized due to the fusion of the foldon trimerizing motif from the T4 fibritin protein. In this case, the duality of targeting and shielding via pIX incorporation of an antibody-related molecule can be examined in manner analogous to that described in Example 3 (targeting) and in this example (shielding).

### Example 5

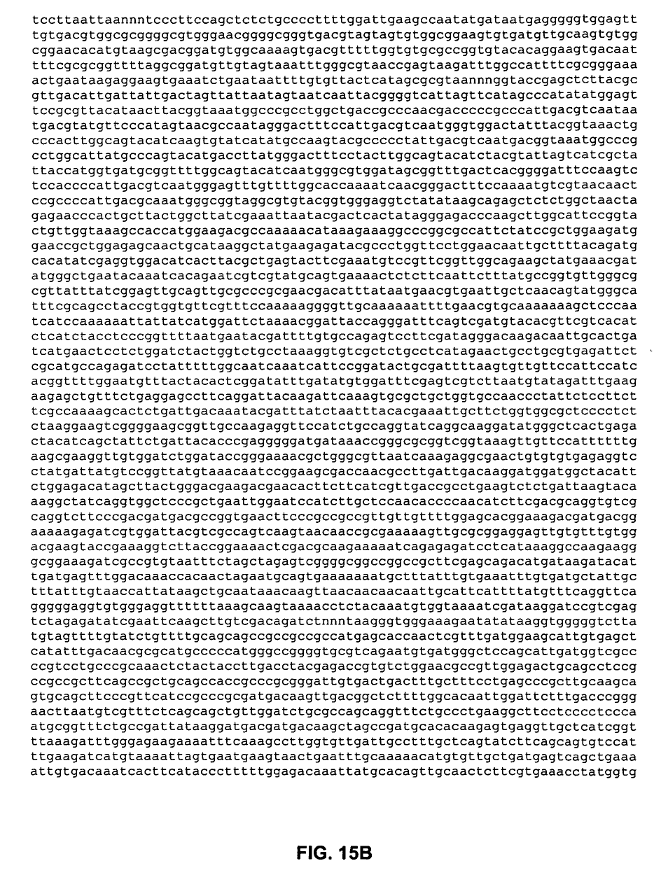

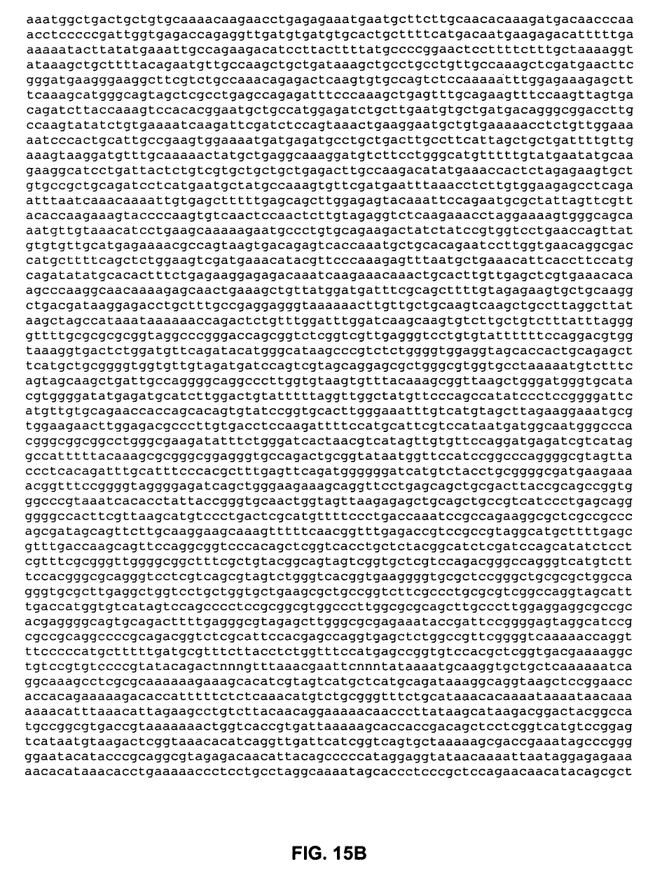

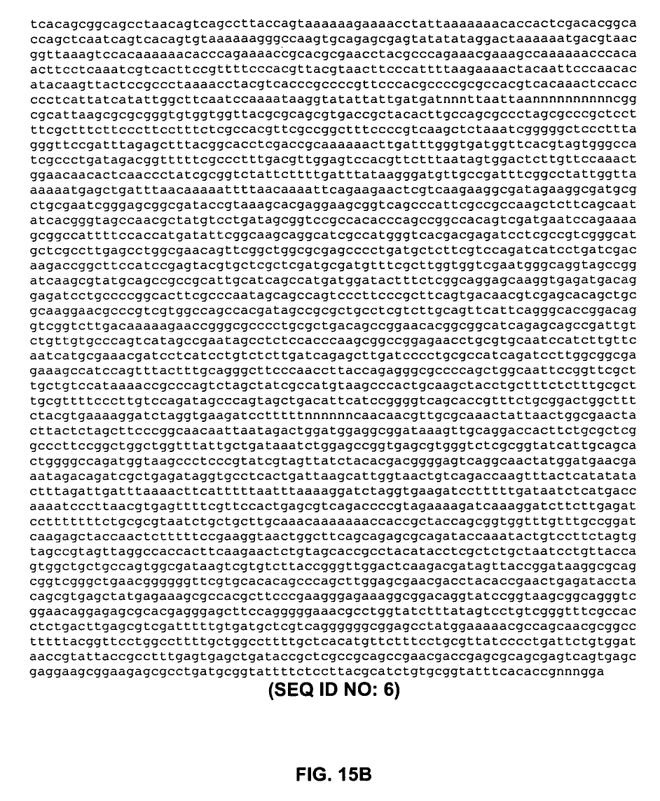

This example demonstrates the construction of an adenovirus incorporating human albumin into the coat protein of pIX (see FIG. 15).

Albumin is a self-protein that is abundant in the body as a serum protein, and is a large monomoeric non-gylcosylate polypeptide with wide in vivo distribution, long half-life and lack of substantial immunogenicity (Peters T. All about albumin: biochemistry, genetics, and medical applications. 1996, San Diego: Academic Press). Several proteins have been fused to albumin to enhance circulating half-life and improve stability for therapeutic applications, including, human growth hormone-rHSA (Albutropin) (Osborn et al. (2002) Eur J Pharmacol 456: 149-158), recombinant granulocyte colony stimulating factor-rHSA (Albugranin) (Halpern et al. (2002) Pharm Res 19: 1720-1729), and serum albumin-CD4 genetic conjugate (Yeh et al. (1992) Proc Natl Acad Sci USA 89: 1904-1908). In addition it has a safe record in clinical practice, for example as exemplified by Albuferon™ (albumin-interferon alpha), which has completed phase II clinical trials by Human Genome Sciences (http://www.hgsi.com/products/albuferon.html).

A further attractive feature of albumin is the modular structure of the protein. Albumin has three homologous domains, each of which have two subdomains (Peters T. All about albumin: biochemistry, genetics, and medical applications. 1996, San Diego: Academic Press) and the crystal structure of albumin has now been realized at 2.5 Å resolution (Sugio et al. (1999) Protein Eng 12: 439-446). Expression of the recombinant domains and even subdomains have been reported, in relation to determining binding sites of warfarin enantiomers (Dockal et al. (1999) J Biol Chem 274: 29303-2931; Dockal et al. (2000) Protein Sci 9: 1455-1465; Twine et al. (2003) Arch Biochem Biophys 414: 83-90) and it has recently been described that domain I of albumin has the most potential as a drug delivery protein carrier (Matsushita et al. (2004) Pharm Res 21: 1924-1932). Therefore it would also be of interest to explore in the context of fusion to pIX, the domains and subdomains of albumin as shielding molecules.

The cDNA of human albumin, a protein consisting of 584 amino acids, is cloned into the NheI site of the previously described shuttle vector pSILucIXNhe. PCR methods generate the cDNA to contain NheI ends to allow for insertion. The human albumin cDNA is present in a commercially available plasmid (Origene). The resultant shuttle plasmid containing albumin fused to pIX is linearized with PmeI digestion to allow homologous recombination with the Ad genome in E. coli using standard methodologies with the commercially available AdEasy system. Viruses, which contain the wild type Ad5 fiber, are propagated in 293 cells, and purified by double cesium chloride ultracentrifugation as standard, then dialyzed against 10 mM Tris buffer, with Mg²⁺, Ca²⁺ and 10% glycerol.

In vitro and in vivo antibody evasion assessment of an Ad vector incorporating human albumin into the pIX coat protein is performed as described in the previous examples.

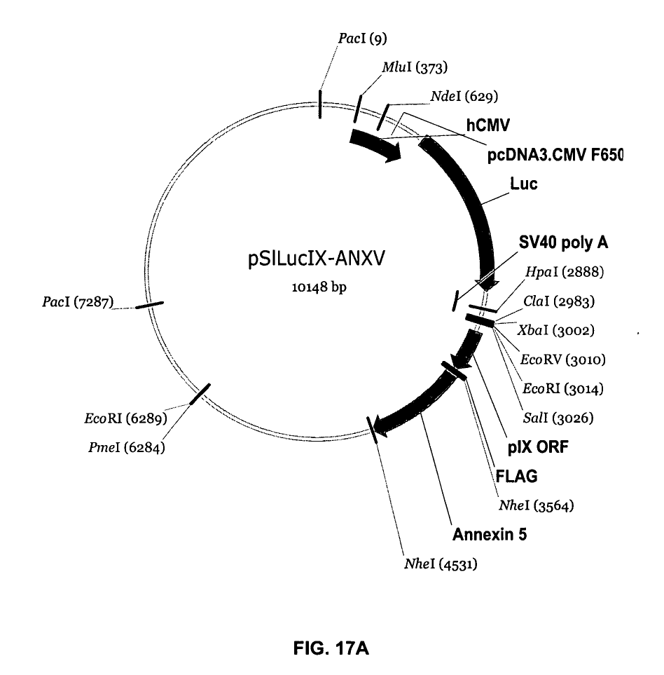

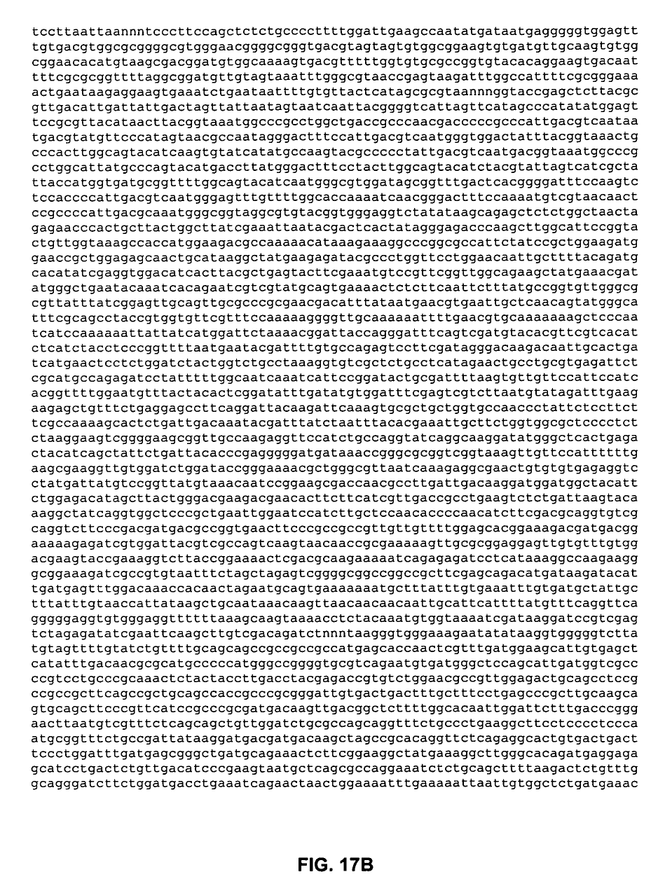

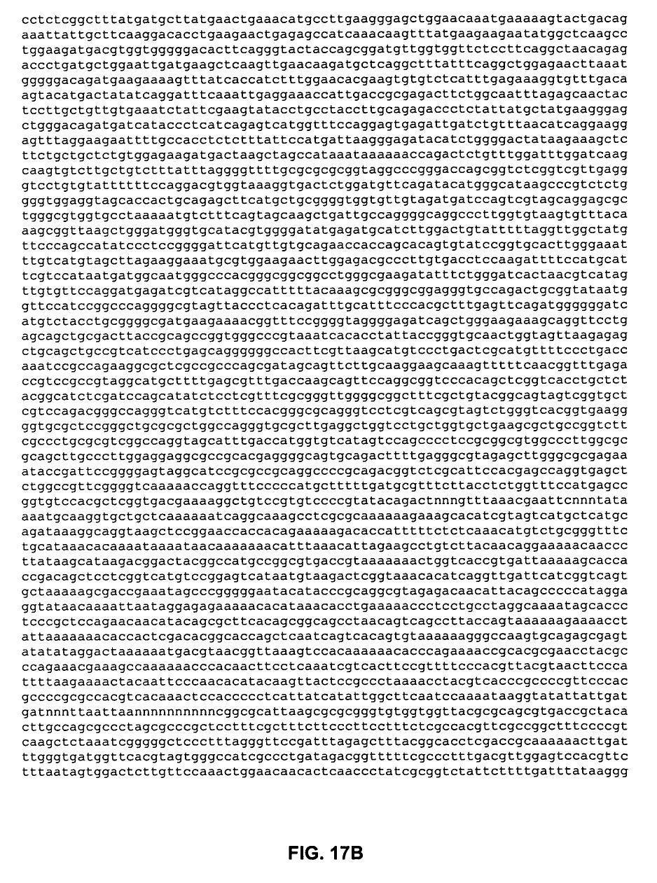

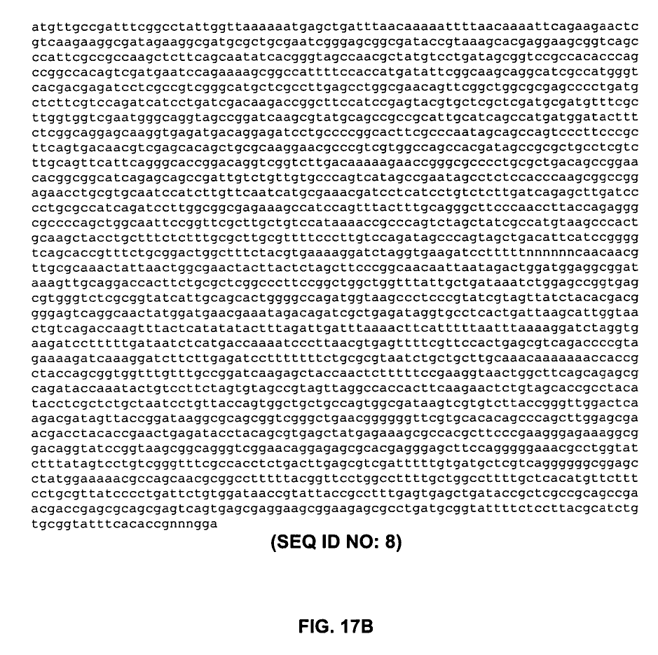

In a further embodiment of this example, alternate self-proteins, either serum or even cytosolic could be fused directly to pIX. These could include, but are not limited to, myglobin, alpha-1-antitrypsin and annexin V (see FIG. 17 for annexin V). Annexin V is a cytosolic protein which can also be detected in serum.

### Example 6

This example presents an in vitro experiment to analyze concept that ligands fused to pIX can provide a shielding effect against neutralizing antibodies.

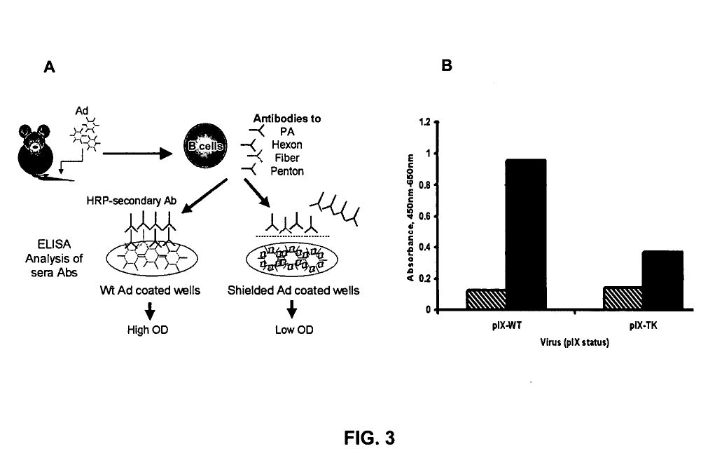

A simple ELISA methodology was employed to analyze the concept of shielding effects via ligands fused to pIX capsid protein. For this preliminary experiment, a virus with the HSV thymidine kinase (TK) protein fused to pIX was utilized. Serum from mice pre-immunization and post-immunization with wild type Ad 5 serotype virus was used as control serum and source of neutralizing antibodies respectively. The idea behind this experiment was to investigate detection of immobilized virions by these antibodies. As the data in FIG. 3 demonstrates, fewer antibodies bound the pIX-TK virus from the post-immunized serum than the non-modified virus (by virtue of the lower OD reading with pIX-TK), indicating virion epitopes recognized by the antibodies were reduced due to the presence of TK on pIX. This experiment demonstrates that ligands fused to pIX provide a shielding effect.

### Example 7

This example represents the use of docking molecules to conjugate shield molecules to pIX.

Cloning of albumin-binding domains (ABD and PAB) and albumin into shuttle vector. ABDs are small amino acid (aa) domains of bacterial origin. The albumin binding domains from streptococcal Protein G (46aas) and P. magnus protein PAB (45aas) are cloned. For generation of cDNAs of ABD or PBA for cloning, two single-stranded oligo nucleotides, one starting at the 5′ end, the other starting at the 3′ end, with an overlapping central 15 nucleotide region, are synthesized. The oligos are annealed and using TAQ extended to form a double stranded (ds) cDNA of ABD, with NheI restriction sites at both the 5′ and 3′ ends. The ds cDNA is digested with NheI, purified and then ligated into pSI.Luc.IX.NheI. PCR and sequencing confirm the correct orientation of these binding domains.

### Example 8

This example demonstrates the development of a shielded adenovirus vector for use as a vaccine platform.

In this embodiment the adenovirus vector would be used as a vaccine against anthrax (for reasons described below), but it is not limited to this pathogen and the shielded vector could be used against plague, Ebola and other emerging infectious diseases.

The civilian population is at high risk from the emerging threat of bio-terrorism, whereby biological agents are used to produce illness or intoxication. The protection against these biological weapons is a major challenge through standard prophylactic vaccine means. The recent anthrax attacks highlighted the urgent need to develop not only therapeutic strategies that act rapidly post-exposure to the biological pathogen, but also rapid acting preventive vaccines that can protect widespread populations at danger (see, e.g., Inglesby et al., (2002) Jama 287: 2236-2252). In addition these vaccine platforms need to be produced in an economically effective manner. Anthrax, which can manifest as cutaneous, gastrointestinal or pulmonary disease, is caused by infection with Bacillus anthracis (see, e.g., Mock & Fouet (2001) Anthrax. Annu Rev Microbiol 55: 647-671). While B. anthracis secretes three proteins, protective antigen (PA), lethal factor (LF) and edema factor (EF), it is the central role of PA in the virulence of this pathogen that makes PA a target for therapies and vaccines. Through the combination with LF and EF, to form exotoxins, systemic fatal pathophysiology occurs (see, e.g., Mock & Fouet (2001) Anthrax. Annu Rev Microbiol 55: 647-671). PA elicits a strong immune response and is used in the current US prophylactic vaccine based on an aluminum hydroxide-adsorbed cell-free filtrate of an attenuated, nonencapsulated strain of B. anthracis (see, e.g., Puziss & Wright (1963) J Bacteriol 85: 230-236). However, the current vaccine requires several administrations over a long time period, a requirement too protracted for use in response to a biological attack with anthrax. The development of an alternate prophylactic vaccine approach, in which the immune response is rapidly mounted, would be of beneficial utility. One such mechanism would be gene delivery of PA, and adenovirus gene delivery vectors provide an ideal platform to meet both economic and vaccination requirements.

Ad vectors, based on the human serotypes 2 & 5, due to their safe clinical profile, and the effective immune response generated against transgenes incorporated into their genomes are promising candidates as prophylactic vaccines (reviewed in Tatsis & Ertl (2004) Mol Ther 10: 616-629). The development of Ad vectors as vaccine delivery vehicles for diseases such as HIV, Ebola and Malaria has progressed rapidly, generally using a prime-boost strategy (see, e.g. Sullivan et al. (2000) Nature 408: 605-609, Shiver et al., (2002) Nature 415: 331-335, Gilbert et al. (2002). Vaccine 20: 1039-1045, Tritel et al., (2003) J Immunol 171: 2538-2547 and Sullivan et al., (2003) Nature 424: 681-684). Indeed, their deployment within the biodefense realm (reviewed in Boyer et al. (2005) Hum Gene Ther 16: 157-168) is currently being explored specifically for anthrax, using single dose strategies with Ad5 vectors encoding PA (see, e.g., Tan et al., (2003) Hum Gene Ther 14: 1673-1682) or EF (see, e.g., Zeng et al., (2005) Vaccine Sep 9; [Epub ahead of print]) to achieve protection or alternatively Ad5 encoding anti-PA antibody to attain passive immunity (see, e.g., Kasuya et al., (2005) Mol Ther 11: 237-244). However, as already described their efficacy within the clinic has a potentially confounding limitation that of the humoral response to Ad vectors. Within the human population there are high titers of pre-existing neutralizing antibodies against Ad5 and Ad2 serotypes (see, e.g., Vogels et al. (2003) J Virol 77: 8263-8271) due to the general exposure to Ads, an issue that has also been discussed within the Ad vector anthrax studies. This limitation means that effective repeat administration of Ad vectors to most tissues is hindered by a strong neutralizing antibody response to the vector. Prime-boost strategies using Ad vector vaccine approaches, generally prime with DNA and then boost with Ad5, rather than prime and boost with Ad5 as re-administration of Ad5 vectors was ineffective (see, e.g., Yang et al., (2003) J Virol 77: 799-803) corroborating with earlier studies of Ad5 re-administration (see, e.g., Kass-Eisler et al., (1996) Gene Ther 3: 154-162 and Chirmule et al., (1999) J Immunol 163: 448-455). Thus far skeletal muscle was one of the few tissues where repeat Ad vector administration was successfully demonstrated (see, e.g., Chen et al., (2000) Gene Ther 7: 587-595). However, the success of this procedure was highly dependent on the initial dose of Ad used in the experiment and therefore, it is still expected that repeat dosing in human is problematic.

Therefore with respect to this Example, the generation of a shielded Ad vector for utility as a vaccine is described as a means to overcome the humoral response to Ad vectors.

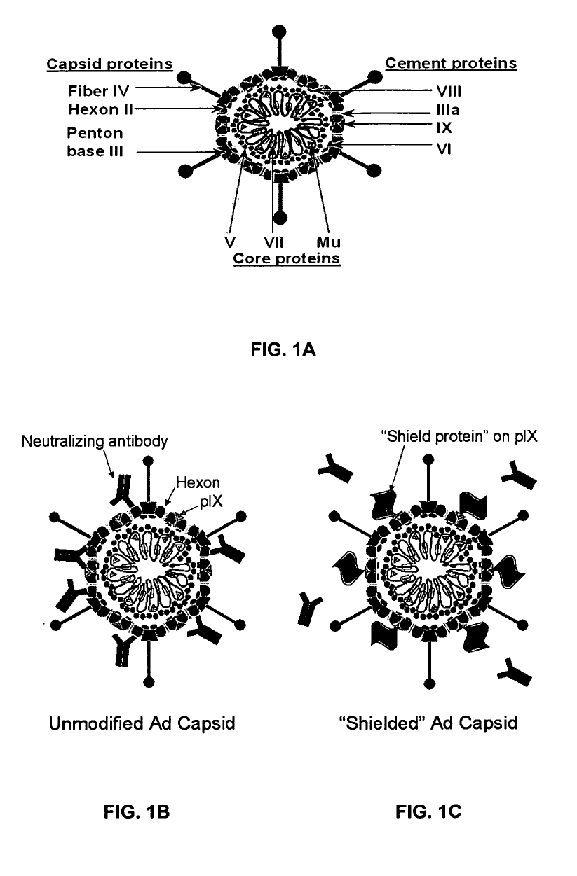

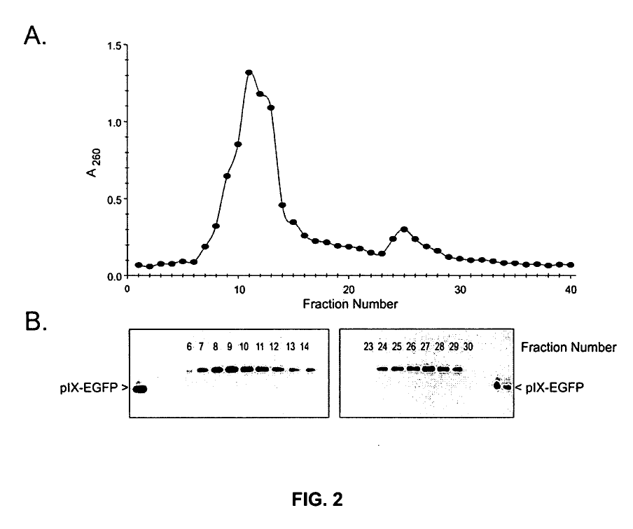

Generation and characterization of Ad vectors expressing anthrax protective antigen with shielding molecule on capsid protein pIX. In the first instance, as direct genetic incorporation of the shielding molecule is most desirable due to the simplicity of a one component system, albumin is analyzed as a suitable shielding protein incorporated at the optimal capsid protein, the pIX locale (as indicated in FIGS. 1B and 1C). Furthermore, albumin binding domains such ABD-3 from Steptococcal protein G and protein PAB of P. magnus, are incorporated and used as docking proteins for the attachment of albumin to coat the virus. These studies will demonstrate the use of a shielded Ad vector for vaccine purposes for biodefense.

TABLE 1
Vector design for transgene (in E1 region) and pIX modification.
A total of 10 vectors are constructed, 5 containing Luc, and 5
containing PA. These vectors have wild type (WT) pIX or for concept 1,
have human albumin (hALB) or murine albumin (mALB) fused to the C
terminus of pIX and for concept 2, have albumin binding domains from
Streptococcal Protein G (ABD) or P. magnus protein PAB (PAB).
Concept 2 vectors are conjugated with albumin from various species,
e.g. human and murine for experimental purposes.

| | PIX-Ligand | | |
|---|---|---|---|
| Transgene | Control | Concept 1 | Concept 2 |

| | | | | | |
|---|---|---|---|---|---|
| Luciferase | WT | hAlb | mAlb | ABD | PAB |
| (Luc) | | | | | |
| Protective | WT | hAlb | mAlb | ABD | PAB |
| Antigen (PA) | | | | | |

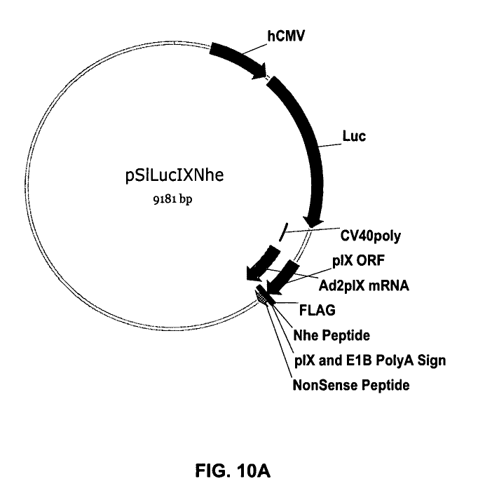

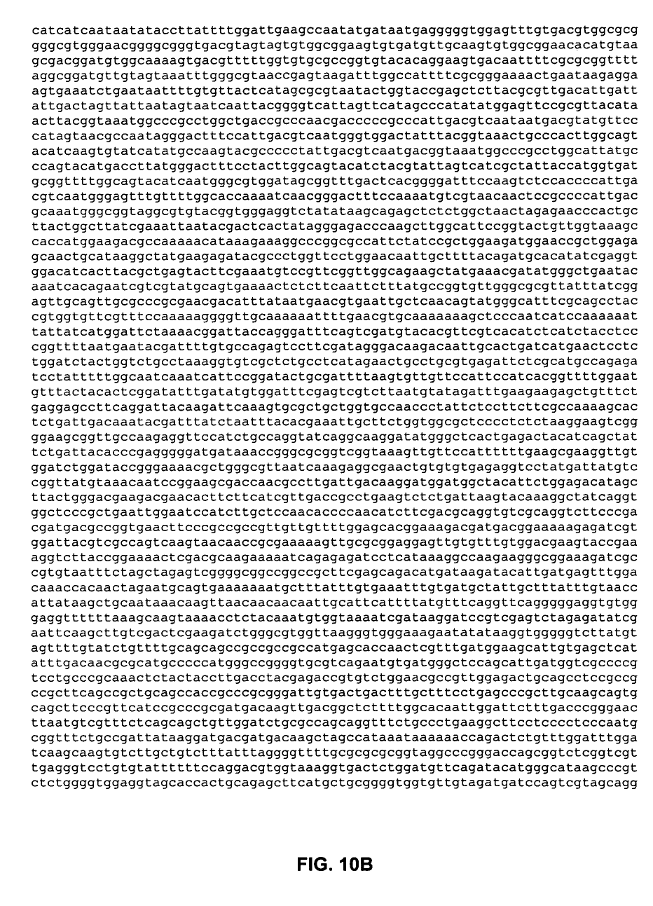

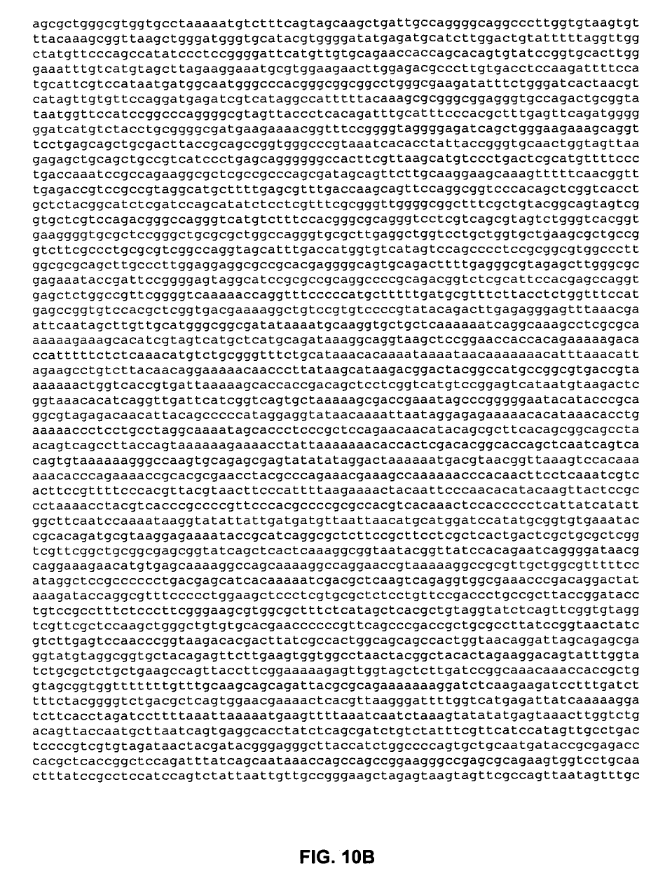

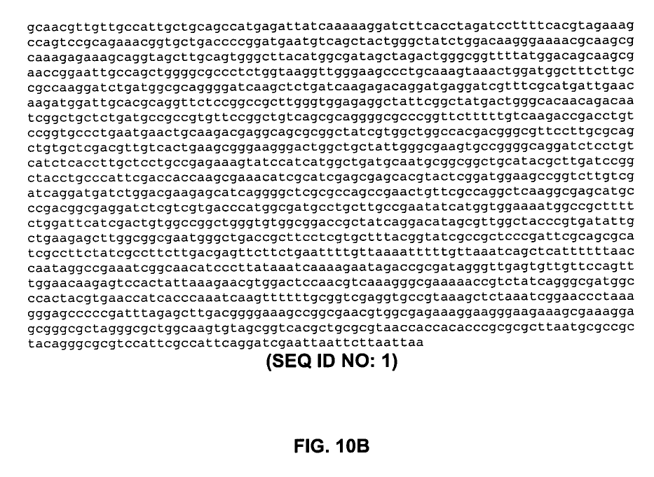

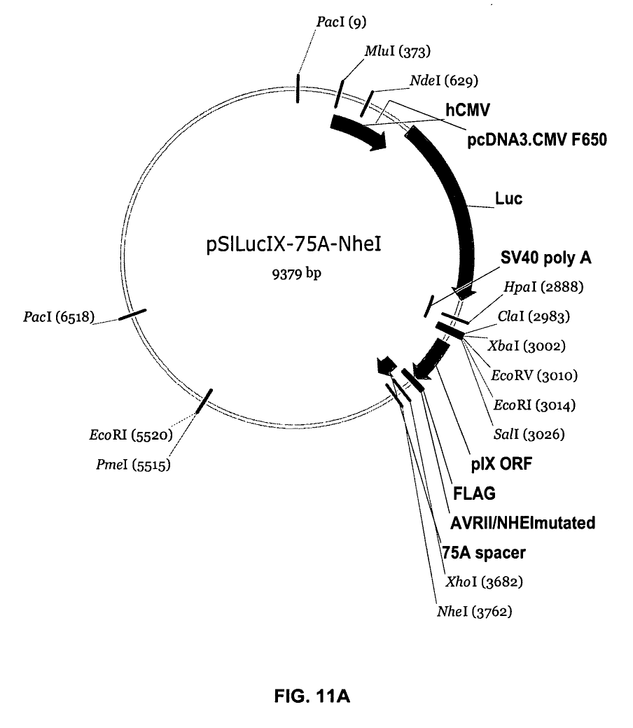

Cloning of PA into shuttle vector. The cDNA for PA has already been cloned and a codon-optimized synthetic form can be optained commercially from PlanetGene. The PA gene is subcloned into pSI.Luc.IX.NheI (see, e.g., Dmitriev et al. (2002) J Virol 76: 6893-6899, see FIG. 10), replacing the Luc gene. This resultant pSI.PA.IX.NheI is the sister shuttle vector for pSI.Luc.IX.NheI and both shuttles can be treated in the exact same manner for subcloning proteins fused to pIX. Both plasmids are linearized with NheI digestion in preparation for protein incorporation. The cloning of the albumin binding domains is performed as described in Example 7. The cloning of human albumin is described in Example 6 and the cloning of mouse albumin will proceed as described here, Mouse albumin (mAlb) is generated using RT-PCR methods. Mouse hepatoma cells, HEPA1-6 (ATCC) are known to produce albumin. These cells are cultured, and mRNA extracted using RNAeasy Kit (Qiagen). Standard RT methods (Omniscript RT, Qiagen) generate cDNA using random hexamers (IDT) and then PCR is used to generate mAlb with NheI ends to allow subcloning into the two shuttle vectors. Prior to the subcloning the hAlb and mAlb fragments are NheI digested and purified. PCR and sequencing confirm the correct orientation of hAlb and mAlb once ligated in the shuttle vectors.

Generation of recombinant Ad vectors. The generated shuttle vectors are PmeI digested so that they can be recombined with pAdEasy1 backbone. The resultant recombinant Ad genomes are checked with PCR methods and once confirmed, digested with PacI to release the viral genome and used to transfect 293 cells in order to rescue the appropriate adenovirus. Standard methods for propagation and CsCl purification of virions are undertaken. In addition, pIX-ABD and pIX-PAB Ad vectors, once CsCl purified and initial pIX incorporation validated (see below), while be conjugated with albumin (human or mouse), through a 1 hour incubation at RT. Following conjugation, viruses are CsCl purified using standard methods. For all vectors standard protein analysis is used to determine viral particles/ml (i.e., particle unit (pu)) and infectivity using the following fluorescent focus assay, in order to determine viral prep quality (viral particle to infectivity ratio).

Fluorescent focus assay. Essentially virus is serially diluted (as serial 10-fold dilutions to 10⁻⁴, 10⁻⁵, 10⁻⁶) and monolayers of 293 cells infected for 60-90 minutes before viral solutions aspirated. Cells are cultured for 48 hours in standard growth medium before medium is aspirated and the cells are washed in PBS and fixed in cold 90% methanol for 10 minutes at room temperature. Wells are washed in PBS and then 0.5 ml of diluted goat anti-adenovirus-FITC antibody (dilute 1:100 in PBS, Chemicon) is added for 30-45 minutes at room temperature. Wells are washed with PBS and then examined under the microscope. Titer is calculated on the basis of number of stained cells per field (need to count an average of 10 fields) and optical properties of the microscope.

Western blot analysis for the presence of modified pIX proteins. The presence of pIX-shield proteins in the context of assembled Ad virions is validated by western blot analysis. Virions harvested from infected cells are purified using standard CsCl gradient centrifugation and 5×10⁹ pu of virus are denatured, per sample, by boiling in Laemmli loading buffer. The viral capsid proteins are separated by a 4-20% gradient polyacrylamide gel (Bio-Rad). The following control viruses are used, Ad5Luc (pIX wild type), and AdLucIXpK (see, e.g., Dmitriev et al. (2002) J Virol 76: 6893-6899), and the electrophorectically resolved viral capsomers are transferred to polyvinylidenedifluoride (PVDF) membrane and probed with anti-pIX monoclonal antibody (1:2000 dilution; ICN Biomedicals Inc.). The blots are developed with the WesternBreeze immunodetection system (Invitrogen) according to manufacturer's protocol.

The purified virions of Ad.Pa.IX, Ad.Pa.IX-ABD, Ad.Pa.IX-hAlb and Ad.Pa.IX-mAlb are used to transduce A549 cells, a cell line that has high expression of the coxsackie and adenovirus receptor (CAR). At various timepoints following transduction, 24-72 hours, cells are harvested and lysed, using cell specific lysis buffer (Promega) for western blot analysis of PA protein content. 50 μg of total protein is mixed with Laemmli loading buffer, loaded and separated by a 4-20% gradient polyacrylamide gel (Bio-Rad). Following transfer to a PVDF membrane, the samples are probed with an anti-PA monoclonal antibody (Abcam, Cambridge, UK). The blots are developed with the WesternBreeze immunodetection system (Invitrogen) according to manufacturer's protocol. PA migrates to approximately 83 kDa.

To examine the ability of the vectors to evade neutralizing antibodies, an in vitro gene transfer assay is used as described in previous examples.

The evaluation of immune response to shielded adenovirus vectors in mice is presented below.

Experiment 1. Assessment of optimal dose for Ad vector administration by intramuscular (i.m.) injection. C57BL/6 female 6-8 wk old mice are purchased from The Jackson Laboratory (Bar Harbor, Me.) and housed under pathogen-free conditions. Animals are treated with one administration of the experimental vectors in comparison to control vector. Initially Ad.Luc vectors are used, but in subsequent experiments Ad.PA vectors are assessed. Administrations actually involve two intramuscular (i.m.) injections, one on either quadricep. The Ad vectors are prepared in 50 μl at the specific dose in saline for vaccinations. Vector dose is at 10⁹, 10¹⁰ or 10¹¹ pu. For all experiments described (Exps 1-4), 10 animals per group are used. Following administration of Ad vectors, animal survival is assessed over a 4 week period, with mice bled from the tail vein at 1, 2 and 4 weeks for analysis of anti-Ad antibodies in mouse sera. Samples are stored appropriately until analysis. In addition, some animals receiving Luc containing vectors are sacrificed at various timepoints to assess biodistribution of the Ad vectors. Analytical methods are described below.

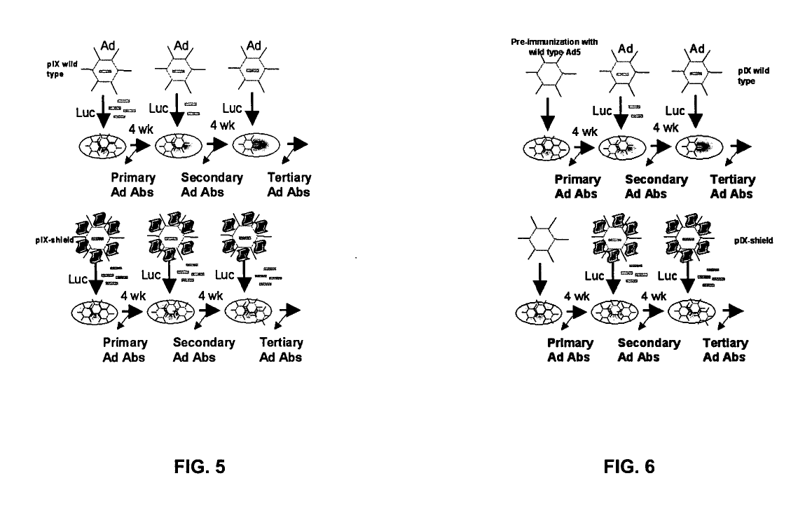

Experiment 2. Determination of the effect of pIX-modification on anti-Ad vector titers. Using the optimal dose determined from experiment 1, C57BL/6 mice receive multiple administrations of the control and experimental vectors (as described for Exp 1) over an 8 week period (see FIG. 5), to assay primary, secondary, tertiary antibodies against the Ad vectors. This experiment only uses Ad.Luc vectors, with the following pIX capsid proteins, pIX-wt, pIX-hAlb, pIX-mAlb, pIX-PAB::hAlb and pIX-PAB::mAlb. Mice are bled from the tail vein and samples stored appropriately until analysis. The following analysis (described in detail below) is conducted on the sera using ELISA methodologies: (a) anti-Ad antibodies and antibody type, IgGs, IgMs are assessed (b) specific virus component antibodies are assessed and (c) whole bound virus are examined for ability to bind or avoid antibodies in the sera. In addition Luc is assayed in some animals.

Experiment 3. Assessment of immune evasion of Ad vectors with modified pIX in Ad5-immunized mice. This experiment mimics the human situation whereby pre-existing neutralizing antibodies against Ad vectors exist. Therefore the experimental vectors are compared with control vector in naïve mice and Ad5-immunized mice (see FIG. 6). The most suitable experimental vectors are used, based on the results in experiments 1 and 2. Mice are pre-immunized with a wild type Ad5 vector through i.m. administration (as previously described). An optimal dose of vector determined from previous experiments is used. 4 weeks following pre-immunization, animals are vaccinated with the control and experimental vectors at the optimal dose decided from experiment 1. In some groups a second administration takes place a further 4 weeks later. Blood samples are taken to analyze for anti-Ad antibodies and also look for the persistence of luciferase expression in the tissues of the mice.

Experiment 4. Development of anti-PA antibodies titers in naïve vs pre-Ad5-immunized mice. Essentially this is a repeat of Experiment 3, but the PA vectors are used to assess anti-PA response in C57BL/6 mice. Mice are treated as naïve, or pre-immunized with Ad5 vector, as previously described for Experiment 3. Mice are vaccinated once or twice (with the second administration being 14 days after the first) with control or experimental vectors (the most appropriate vector/vectors as determined from experiments 1-3 is used), and then are bled at 1, 2, and 4 weeks after vaccination to assess anti-PA immunity. Mice are bled from the tail vein, samples centrifuged and sera stored at −20 C until assayed for anti-PA antibodies by ELISA as described below. In addition, analysis of the immune response against the Ad vector is monitored as previously described.

Antibody production against Ad vector in mice. ELISA plates are covered with a goat anti-mouse IgG (IgG) (1:500) for measurement of total IgG antibodies within mouse sera. For individual subtypes of Igs, plates are covered with wild type Ad virus. The wells are washed and then blocked with 3% BSA at room temperature for 2 h. Serum is diluted with 3% BSA at 1:3000 for assay of total IgG or 1:500 for assay of individual Igs and incubated for 30 minutes at 4 C. After washing, horseradish peroxidase (HRP)-conjugated goat anti-mouse IgGs for total IgG assessment and class specific immunoglobulins, IgG1, IgG2b, IgG2c (B6 isotype), IgG3 and IgM are added to the appropriate wells, followed by further washing and color development with tetramethylbenzidine (TMB, Sigma) substrate. The plates are read at 450/650 nm using a microplate reader (Emax; Molecular Devices, CA).

Capsid component antibody production against Ad vector in mice. Purified recombinant capsid proteins, hexon, penton, fiber or pIX, (with 6-His tags for purification purposes) are coated at 100 μl of 5 μg/ml on a 96 well plate for overnight incubation at 4 C.

The following day wells are washed with PBS/Tween-20 and then blocked with 3% milk/1% PBS for one hour. After washing 4 times with PBS/Tween20, serum from the experimental animals (including serum from naïve animals) is added at 1:10 (using 3% milk/PBS to dilute) and plates incubated at room temperature for 2 hours. Wells are then washed 6 times with PBS/Tween20 and HRP conjugated goat anti-mouse IgG (diluted 1:5000 in 3% milk/PBS) is added for 1 hour at room temperature. After washing 6 times, TMB is added, and after 10 minutes maximum the reaction is stopped with 0.4N H₂SO₄. Plate is read at 459/650 nm as described above.

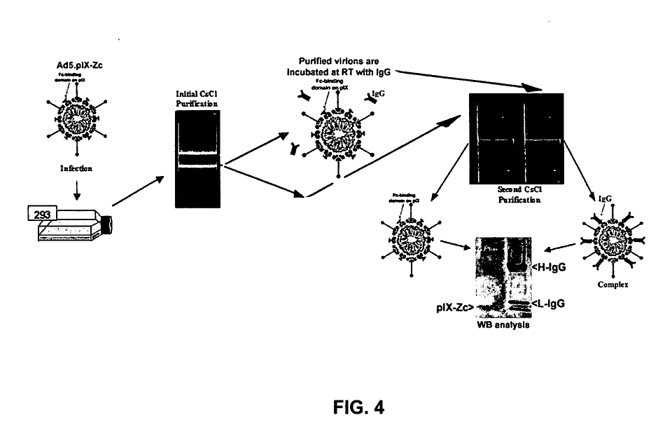

Recognition of Ad vectors with sera from immunized mice. As in the preliminary data (FIG. 4), virions from modified pIX viruses or control wild type pIX virus are coated on a 96 well plate for overnight incubation at 4 C. The following day wells are washed 4 times with PBS/Tween-20 and then blocked with 3% milk/1% PBS for one hour. After washing 4 times with PBS/Tween20, serum from the experimental animals (including serum from naïve animals) is added at 1:10 (using 3% milk/PBS to dilute) and plates incubated at room temperature for 2 hours. Wells are then washed 6 times with PBS/Tween20 and HRP conjugated goat anti-mouse IgG (diluted 1:5000 in 3% milk/PBS) are added for 1 hour at room temperature. After washing 6 times, TMB is added, and after 10 minutes maximum the reaction is stopped with 0.4N H₂SO₄. Plate is read at 459/650 nm as described above.

Analysis of luciferase expression in mice administered with pIX-modified Ad vectors. Mice are euthanized via standard protocols at the appropriate timepoints to allow examination of the transgene expression within the muscle and a control tissue, such as lung or liver. Organs are excised and stored at −80 C until further analysis. Frozen organs are ground to a fine powder using a mortar and pestle, and then cooled in a dry ice-ethanol bath. Organ powders are lysed using Cell Culture Lysis Buffer (Promega) at room temperature for 20 minutes. Lysates are frozen and thawed once, and then centrifuged at 14000 rpm for 15 minutes in a tabletop Eppendorf centrifuge. Luciferase activity in 1:20 diluted samples is measured using the Luciferase Assay System (Promega) according to the manufacturer's instructions. Luciferase values are normalized for protein content, as determined by the Bio-Rad DC Protein Assay system (Bio-Rad, CA).

Antibody production against protective antigen in mice. ELISA is used to analyze the production of anti-PA antibodies the mouse sera essentially as described (see, e.g., Tan et al., (2003) Hum Gene Ther 14: 1673-1682) with minor modifications. Flat-bottomed 96-well plates are coated with PA antigen (100 μl/well of 1 μg/ml PA) overnight at 4 C. The wells are washed and blocked with 5% dry milk in PBS for 30 minutes at room temperature. After washing in PBS, serial dilutions of serum are added to each well for 1 hour at room temperature. Plates are washed with PBS-Tween 20 (0.05%) and then goat anti-mouse immunoglobulin antibodies conjugated with horseradish peroxidase (HRP) (Dako Corporation, Carpinteria, Calif.) applied and the color is developed with the Sigma FAST o-phenylenediamine dihydrochloride tablet kit (Sigma, St Louis, Mo.) as recommended by the manufacturer. The color intensity is measured at 490 nm with an EL800 plate reader (Bio-Tek Instruments, Winooski, Vt.).

Efficacy of shielded Ad vectors in prophylactic protection of rabbits to inhalation of anthrax spores. The employment of a suitable animal model in which to study the prophylactic effect of the shielded Ad vectors against anthrax, which have been developed and characterized in the first two aims of the Example, is very important in realizing the aim of creating a shielded Ad vector vaccine. In previous studies Ad vector efficacy in providing protection or generating passive immunotherapy against anthrax has been assessed against lethal toxin administration in mice models (see, e.g., Tan et al., (2003) Hum Gene Ther 14: 1673-1682, Kasuya et al., (2005) Mol Ther 11: 237-244, Hashimoto et al., (2005) Infect Immun 73: 6885-6891) or through exposure of mice to non-capsulated spores, such as B. anthracis Sterne strain (see, e.g., Zeng et al., (2005) Vaccine Sep 9; [Epub ahead of print]). One essential flaw with mouse models, the mode of death is radically different in mice to other animal models. Essentially the capsule component of the anthrax spore kills the mouse before lethal toxin and edema can be produced (see, e.g., Welkos et al., (1986) Infect Immun 51: 795-800, Welkos & Friedlander, (1988) Microb Pathog 5: 127-139, Welkos et al., (1989) Microb Pathog 7: 15-35) whereas it is the lethal toxin and additional effects of edema, which kill most animal species including humans (see, e.g., Phipps et al., Microbiol Mol Biol Rev 68: 617-629). In addition, different strains of mice have variable response to lethal toxin (see, e.g., Welkos et al., (1986) Infect Immun 51: 795-800), and this further complicates the comparison of studies, although administration of lethal toxin per se can provide information about how the immune system responds or protects against the developing disease. While the outcome of Ad studies suggest mouse models can be correlated to anthrax pathobiophysiology (see, e.g., Tan et al., (2003) Hum Gene Ther 14: 1673-1682, Zeng et al., (2005) Vaccine Sep 9; [Epub ahead of print], Kasuya et al., (2005) Mol Ther 11: 237-244, Hashimoto et al., (2005) Infect Immun 73: 6885-6891) the actual mimicking and thus correlation of subsequent disease progression from the most probably route of B. anthracis infection through inhalation of anthrax spores from a bio-attack in humans is not possible in mice. Therefore, while immune characterization of the Ad vectors, and proof of principle studies to demonstrate the concept of shielding of Ad vectors are performed in mice, lethal toxin studies are not be done in mice and instead the most appropriate inhalational rabbit model of anthrax (see, e.g., Phipps et al., (2004) Microbiol Mol Biol Rev 68: 617-629) is used.

To date, anthrax vaccination or therapy against anthrax exposure with gene delivery through Ad vectors have not yet been studied in the rabbit, but this model has been shown effective for passive immunotherapy of inhalational anthrax, with anti-PA administration in a study (see, e.g., Mohamed et al., (2005) Infect Immun 73: 795-802). Expression of transgene in various rabbit models through various administration routes has been achieved from Ad vectors (see, e.g. Li et al., (2005) J Gene Med 7: 792-802, Mehta et al., (2005) J Hand Surg [Am] 30: 136-141, Wen et al., (2003) Exp Eye Res 77: 355-365). Therefore the most appropriate shielded Ad vector are translated into a vaccine study using New Zealand White rabbits, for an inhalational anthrax model.

Experiment 1: Development of neutralizing Ad antibodies and prophylactic effect of the shielded Ad vector against anthrax challenge. In the first instance neutralizing antibodies are stimulated against Ad5 vectors in a dose dependent manner and then rabbits are vaccinated with one dose of the shielded vector. The rabbits are divided as such: Group A, naïve rabbits unexposed to wild type Ad5 vector (naïve), Group B, rabbits pre-immunized to wild type Ad5 vector (immunized), i.m. administration of 2×10¹⁰ pu or Group C, rabbits pre-immunized to wild type Ad5 vector (immunized), i.m. administration of 10¹¹ pu. 14 days after pre-immunization, each group is subdivided into those receiving (i) PBS control, (ii) Ad.PA.pIX-wt and (iii) Ad.PA.pIX-shield. This is done via i.m. administration at 10¹¹ pu. Rabbits are challenged with anthrax spores 14 days after the administration of Ad.PA vectors. End-point assays include ELISAs for neutralizing anti-Ad antibodies and ELISA for anti-PA antibodies, health and survival. Animals are monitored throughout and in the 28-days following the spore challenge. Testing for the presence of B. anthracis in recently deceased animals is performed. Spore challenge is done using muzzle-only exposure system according to standard procedures. 5 animals per group are used.

| Day 0 | Day 14 | Day 28 | Day 60 |
|---|---|---|---|
| Ad5 immunization | Experimental | Spore challenge | Monitor animals |
| | Vector i.m. | | |

Experiment 2: Effect of multiple dosing on the prophylactic effect of the shielded Ad vector against anthrax challenge. Utilizing the optimal condition for stimulating neutralizing Ad antibodies, multiple dosing is tested to analyze vaccination of the animals against anthrax challenge. Naïve (group A), or immunized rabbits (group B, using optimal dose from experiment 1) are divided into the following subgroups: 1. PBS control, 2. single dose Ad.PA.pIX-wt, 3. double dose (ie boosting) Ad.PA.pIX-wt, 4. single dose Ad.PA.pIX-shield, 5. double dose Ad.PA.pIX-shield. Rabbits are vaccinated with a dose of 10¹¹ pu via i.m administration. For single dose animals, administration is 14 days after pre-immunization, and then in the boosting strategy, the first dose is at 14 days after pre-immunization, with the boosting dose a month later. Anthrax spore challenge would then be a further 14 days later. End-point assays include ELISAs for neutralizing anti-Ad antibodies and ELISA for anti-PA antibodies, health and survival. Animals are monitored throughout and following a 28-day period after spore challenge. Testing for the presence of B. anthracis in recently deceased animals is performed. Spore challenge is done using muzzle-only exposure system according to standard procedures. 5 animals per group are used.

| Day 0 | Day 14 | Day 34 | Day 58 | Day 86 |
|---|---|---|---|---|
| Ad5 | Experimental | Experimental | Spore | Monitor |
| immunization | Vector i.m | Vector i.m. | Challenge | animals |

Antibody production against Ad vector in rabbits. ELISA plates are covered with a goat anti-rabbit IgG (IgG) (1:500) for measurement of total IgG antibodies within mouse sera. For individual subtypes of Igs, plates are covered with wild type Ad virus. The wells are washed and then blocked with 3% BSA at room temperature for 2 h. Serum is diluted with 3% BSA at 1:3000 for assay of total IgG or 1:500 for assay of individual Igs and incubated for 30 minutes at 4° C. After washing, horseradish peroxidase (HRP)-conjugated goat anti-mouse IgGs for total IgG assessment or class specific immunoglobulins for IgGs and IgM are added to the appropriate wells, followed by further washing and color development with TMB. The plates are read at 450/650 nm using a microplate reader (Emax; Molecular Devices, CA).

Antibody production against protective antigen in rabbits. Costar high binding plates were coasted with PA at a concentration of 0.6 μg/ml and incubated overnight at 4 C. Wells are washed and blocked with Superblock reagent (300 μl/well) for 1 h at room temperature. The blocking solution is aspirated and wells allowed to air dry. A starting dilution of 1:100 of each serum is used, (this has previously been shown to prevent any interference in the signal of the assay) and samples are incubated for 30 minutes at 37 C. A goat anti-rabbit IgG HRP conjugate is added for 30 minutes at 37 C and color developed with TMB for 15 minutes at room temperature. The reaction is stopped with 2N H₂SO₄ and plates read at 450 nm. The serum dilution that result in an optical density signal of 1 was used as a measure of the response (titer).

In alternate embodiments of this example, the antigen can be replaced by any antigen of choice, relating to the appropriate disease and could be but not limited to plague, Ebola, etc.

The shielding molecule in alternate embodiments could be a smaller domain of albumin, such domain one of albumin, or an alternate protein such as but not limited to myoglobin, alpha-1-antitrypsin or annexin V.

In a further embodiment of this example, shielding proteins may be extended away from the capsid by spacer peptides, or additional shielding proteins maybe inserted into other capsid proteins, such as fiber, penton, hexon or pIIIa.

### Example 9

This example represents the development of a shielded conditionally replicative adenovirus.

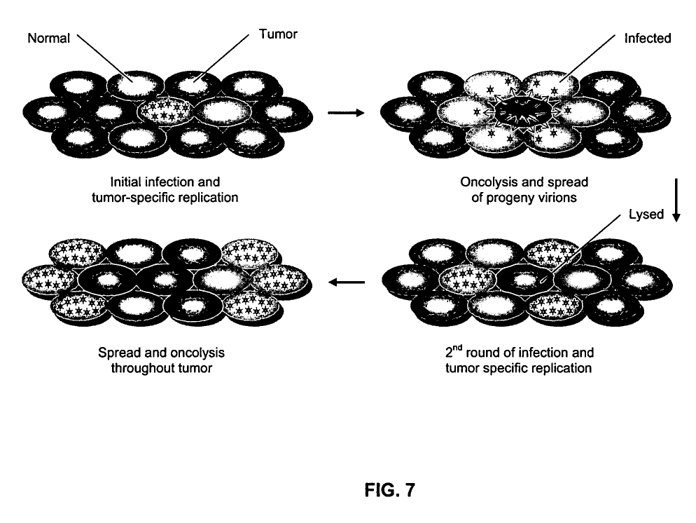

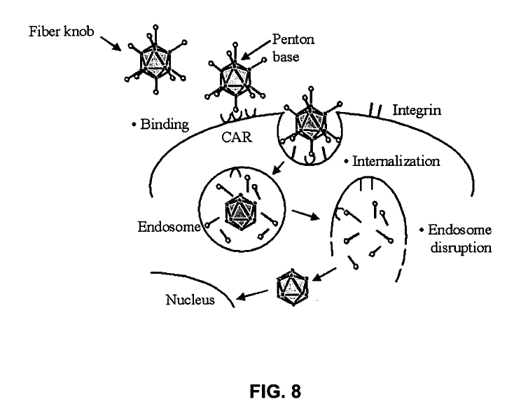

Conditionally replicative adenoviruses (CRAds) are novel vectors with utility as virotherapy agents for cancer gene therapy. Virotherapy, the use of replicative viruses, is a highly attractive approach, pursued to address the problem of limited tumor transduction in particular by adenovirus vectors experienced in earlier cancer gene therapy strategies (Alemany et al. (2000) Nat Biotechnol 18: 723-727 and Kim D et al. (2001) Nat Med 7: 781-787). Virotherapy exploits the lytic property of virus replication to kill tumor cells. Because this approach relies on viral replication, the virus can self-amplify and spread in the tumor from an initial infection of only a few cells (FIG. 7). Although attempted in the past and abandoned because of toxicity and inefficacy (Sinkovics & Horvath (1993) Intervirology 36: 193-214), this “virotherapy” approach has reemerged with great promise in a large part due to better understanding of virus biology and the ability to genetically modify viruses. With this knowledge, researchers can now design viruses to replicate in and kill tumor cells specifically.

Adenovirus is a highly desirable vector for utilization in virotherapy approaches, as this virus has many attractive features such as low pathogenicity for humans, lack of integration in host cell genome and these viruses can be grown to high titers. In addition, they have unique utility for in vivo application due to their high efficacy compared with other approaches (Russell (2000) J Gen Virol 81: 2573-2604, Glasgow et al., (2004) Curr Gene Ther 4: 1-14). However adenovirus does not have natural predilection to replicate in tumor cells, but can be rendered specific for tumor replication through two divergent pathways. In the first instance selective replication is achieved by the regulation of viral genes with tumor-specific promoters. In recent years a plethora of CRAds have emerged harboring the essential E1A gene region under the control of tumor-specific promoters/elements, including the alpha-fetoprotein promoter (Hallenbeck et al., (1999) Hum Gene Ther 10: 1721-1733), prostate-specific enhancer (Rodriguez et al., (1997) Cancer Res 57: 2559-2563), DF3/MUC1 promoter (Kurihara et al., (2000) J Clin Invest 106: 763-771), midkine promoter (Adachi et al., (2001) Cancer Res 61: 7882-7888), tyrosinase promoter/enhancer (Nettelbeck et al., (2002) Cancer Res 62: 4663-4670), and COX-2 promoter (Yamamoto et al., (2003) Gastroenterology 125: 1203-1218). Most of these agents have demonstrated remarkable preclinical results in eradicating tumors in xenograft mouse models.

In the second scheme, selective replication is achieved in theory by the deletion of viral functions dispensable in tumor cells. This was pioneered through the use of a mutant Ad (dl 1520, also known as ONYX-015) that is deleted in the adenoviral ELB-55 kD protein, which normally binds to and inactivates p53. Such a modification was hypothesized to make the virus (ONYX-015) replicate only in p53-defective cells (Bischoff et al., (1996) Science 274: 373-376) (the case in 50% of human tumors); however, this principle has been questioned (Harada & Berk, (1999) J Virol 73: 5333-5344, Hay et al., (1999) Hum Gene Ther 10: 579-590 and Vollmer et al., (1999) Cancer Res 59: 4369-4374). Furthermore, the replication of this virus was severely hampered compared to wild type virus probably due to the late virus mRNA transcription function of the missing ELB-55 kD protein (Vollmer et al., (1999) Cancer Res 59: 4369-4374). Despite the drawbacks realized with the initial ONYX-015 virus the stage was set for the design of improved second generation CRAds that are more selective for tumor cells as already discussed above (and reviewed Davis & Fang, (2005) Journal of Gene Medicine 7: 1380-1389). Of note is the Δ24 adenovirus with a 24 base pair deletion in the E1A gene domain interacting with the retinoblastoma (Rb) protein which was incorporated into a CRAd (Fueyo et al., (2000) Oncogene 19: 2-12 and Heise et al., (2000) Nat Med 6: 1134-1139). However, there is concern that the therapeutic index actually comes from reduced replication potential within non-dividing/slow growing cells (such as normal cells) versus normal replication within fast growing cells, and hence this mechanism is not fully tumor specific (Johnson et al., (2002) Cancer Cell 1: 325-337) leading these researchers to include further modifications. Therefore one should perhaps be guarded about using the term tumor specific replication with respect to these CRAds. Regardless of the terminology used, however many clinical trials have demonstrated safety but limited efficacy, in particular with ONYX-015 (Edelstein M, www.wiley.co.uk/genmed/clinical. 2004, John Wiley and Sons Ltd and Chiocca EA, www.oncolyticvirus.org. 2004) highlighted two confounding limitations, the natural tropism of Ad vectors and the humoral response to Ad vectors. Therefore the development of CRAd vectors would be greatly improved by addressing these two factors.

In the first instance, the paucity of the natural receptor for serotype Ad5 vectors, the coxsackievirus and adenovirus receptor (CAR), on many cancer tissues (e.g. Kim et al., (2002) Eur J Cancer 38: 1917-1926, Miller et al., (1998) Cancer Res 58: 5738-5748, Cripe et al., (2001) Cancer Res 61: 2953-2960, Li et al., (1999) Cancer Res 59: 325-330 and Okegawa et al., (2000) Cancer Res 60: 5031-5036) hinders the efficacy of CRAd virotherapy and therefore the utility of Ad vectors would be further enhanced by re-directing their tropism to alternate receptors. The characterization of the adenovirus entry pathway (FIG. 7) has provided an understanding of the means of modifying of adenovirus tropism. Briefly, cellular recognition is mediated through the globular carboxy-terminal “knob” domain of the adenovirus fiber protein and CAR (Henry et al., (1994) J Virol 68: 5239-5246 and Krasnykh et al., (1996) J Virol 70: 6839-6846) with internalization of the virion by receptor-mediated endocytosis following. This in turn is mediated by the interaction of Arg-Gly-Asp (RGD) sequences in the penton base with secondary host cell receptors, integrins α_{V}β₃ and α_{V}β₅ (Wickham et al., (1993) Cell 73: 309-319). Post-internalization, the virus is localized within the cellular vesicle system, initially in clathrin-coated pits and then in cell endosomes (Wang et al., (1998) J Virol 72: 3455-3458). The virions escape and enter the cytosol due to acidification of the endosomes, which has been hypothesized to occur via a pH-induced conformational change. Essentially this causes an alteration in the hydrophobicity of the adenoviral capsid proteins, specifically penton base, to allow their interaction with the vesicle membrane. Upon capsid disassembly and cytoplasmic transport, the viral DNA localizes to the nuclear pore and is translocated to the nucleus of the host cell (Greber et al. (1993) Cell 75: 477-486).

To develop a truly targeted Ad vector, it is necessary to ablate both native viral tropism and to introduce a novel specificity, which allow infection of the cells of interest via alternative receptors. Genetic modification of the fiber protein and/or other capsid proteins is a rational approach for introducing a novel cell-specific tropism and permit ablation of CAR interaction. Several different approaches can be untaken, including substitution with a fiber or knob of an alternate adenovirus serotype, replacement of the fiber or knob with an alternate trimerization motif to allow large ligand incorporation or simply peptide insertions into the HI loop or C-terminus of the fiber itself (Mathis et al. (2005) Oncogene 24: 7775-91).

In the case of CRAd targeting, though, the specificity to tumor cells achieved through their replication cycle permits the infectivity enhancement approach, whereby for example the inclusion of the RGD motif into the HI loop to direct CRAds to integrins. Several cancer tissues are rich in the expression of appropriate integrins e.g. (Albelda et al., (1990) Cancer Res 50: 6757-6764 and Gladson & Cheresh, (1991) J Clin Invest 88: 1924-1932), whereas low in expression of CAR (You et al., (2001) Cancer Gene Ther 8: 168-175). Such targeting can be combined with replication control to achieve selective or enhanced tumor killing (Suzuki et al., (2001) Clin Cancer Res 7: 120-126) especially for cancers that are deficient in the primary adenoviral receptor (Douglas et al., (2001) Cancer Res 61: 813-817). The combination of viral gene mutation compensation and transductional targeting has led to the development of the AdΔ24-RGD CRAd (Suzuki et al., (2001) Clin Cancer Res 7: 120-126), which has enhanced tumor killing (Bauerschmitz et al., (2002) Cancer Res 62: 1266-1270, Suzuki et al., (2001) Clin Cancer Res 7: 120-126, Lamfers et al., (2002) Cancer Res 62: 5736-5742, Fueyo et al., (2003) J Natl Cancer Inst 95: 652-660, Lam et al., (2003) Cancer Gene Ther 10: 377-387 and Bauerschmitz et al., (2004) Int J Cancer 111: 303-309). These studies thereby demonstrate great promise for the development of CRAds that can achieve safe, selective, and effective tumor eradication. However it is still perceived that the host humoral response potentially limit any gains seen from the infectivity enhancement of AdΔ24-RGD and therefore a strategy to limit vector immunity is required. As already discussed, and further discussed her, in the context of CRAds (Davis & Fang, (2005) Journal of Gene Medicine 7: 1380-1389). Clinical trials utilizing ONYX-015 have highlighted a strong innate immune response in several patients, that was highly suggestive of limited efficacy with the virus (Ganly et al., (2000) Clin Cancer Res 6: 798-806, Nemunaitis et al. (2000) Cancer Res 60: 6359-6366 and Nemunaitis et al., (2001) Gene Ther 8: 746-759). Mathematical modelling of oncolytic adenovirus spread throughout tumor mass has also predicted that the immune response is limiting to viral clearance of the tumor (Wu et al., (2004) Bull Math Biol 66: 605-625). There are numerous potential ways to overcome vector humoral response, but the direct incorporation of a shielding ligand into the pIX capsid protein embodies the most desired strategy to achieve a shielded CRAd vector.

pIX-modified Ads retain viral replication and cytopathic capabilities. Ideally for the application of shielding in CRAds, modification of pIX should minimally disrupt the efficiency of replication and virus production. Protein IX has been shown to play a number of roles in adenovirus infection, including capsid stabilization, transcriptional activity, and nuclear reorganization (Rosa-Calatrava et al. (2001) J Virol 75: 7131-7141). Although dispensable in packaging (Colby & Shenk (1981) J Virol 39: 977-980), adenovirus pIX is important in packaging full-length genomes and stabilizing the capsid structure (Ghosh-Choudhury et al. (1987) Embo J 6: 1733-1739 and Furcinitti et al. (1989) Embo J 8: 3563-3570). The effect of fusing EGFP to pIX on DNA packaging and hence progeny production, viral DNA was quantitated using Taqman quantitative PCR on days 1, 2, 3, and 4 following infection at 10 fcu/cell (fluorescent cell units/cell). This analysis indicated that total viral DNA replication was the same for both Ad-IX-EGFP and control Ad-CMV-EGFP (both E1 deleted vectors assayed on E1 complementing cell lines), but Ad-IX-EGFP had lower progeny yield than Ad-CMV-EGFP, although within similar magnitude. In addition, thermostability was also marginally affected in the pIX-modified vector (Le et al., (2004) Mol Imaging 3: 105-116.

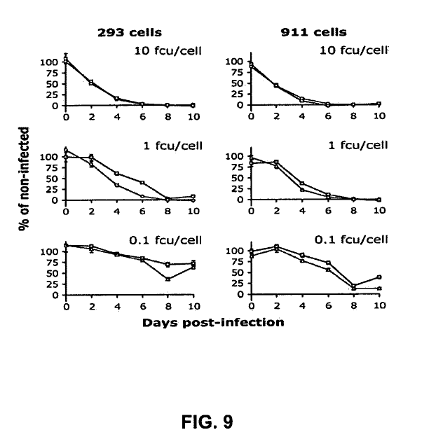

In addition to efficiency of progeny production, CRAd efficacy also depends on how well the virus can lyse infected tumor cells and spread leading to an overall cytopathic effect. To evaluate Ad-IX-EGFP quantitatively for cytopathic effect, infection of 911 and 293 cells with Ad-IX-EGFP and control virus (both E1 deleted) at 10, 1, and 0.1 fcu/cell multiplicities of infection (moi) were monitored over 10 days. On days 0, 2, 4, 6, 8, and 10, the cytopathic effect of the virus was quantitated using a non-radioactive cell proliferation assay (MTS assay) (FIG. 9). Both 293 and 911 packaging cell lines for E1-deleted adenoviruses have been shown to express very low levels of wild-type pIX (Ghosh-Choudhury et al., (1987) Embo J 6: 1733-1739 and Graham et al., (1977) J Gen Virol 36: 59-74). In both 293 and 911 cells, Ad-IX-EGFP cytopathic effect was the same as that of Ad-CMV-EGFP. These findings suggest that although Ad-IX-EGFP has a slightly lower yield than control virus, pIX-EGFP did not affect the cytopathic capacity and lateralization of the virus, critical functions of replicative adenoviral agents. While these data represent somewhat artificial models in which to assess these parameters, replication competent Ad.pIX-TK virus (E1 intact) has been shown to grow to comparable titers of a wild type Ad5 virus and affect CPE in the same manner as the wild type Ad5 (Li et al., (2005) Virology 338: 247-258), indicating that pIX-modification should not significantly hinder CRAd replication, progeny production and cytopathic effect.

In conclusion, this data confirms that the pIX capsid protein is a suitable locale in the adenovirus capsid for genetic modification without hindering viral replication and CPE, while thermostability would probably be marginally affected and therefore shielding of a CRAd vector would be able to proceed.

Experiment 1 Generation and characterization of CRAd, AdΔ24-RGD-pIX-shield (AdΔ24S-RGD), with shielding molecules on capsid protein pIX.

Generation of recombinant CRAd vector with pIX-shield. An AdΔ24S-RGD is generated to contain either human albumin (hALB) or mouse albumin (mALB) genetically incorporated to C terminus of pIX through a FLAG amino acid linker. Control Ad vectors in this study are the non-replicative, Ad.Luc.RGD, original AdΔ24, and the parental AdΔ24-RGD. More in depth details are provided below.

Generation of the shuttle vector to contain pIX-albumin. Prior to the cloning of hALB or mALB into a pIX shuttle vector, the pCX1-Δ24 (Fueyo et al. (2000) Oncogene 19: 2-12) is manipulated to contain the pIX-flag-NheI region for cloning purposes. This is done by PCR methods, using pSI.Luc.IX.NheI (Dmitriev et al. (2002) J Virol 76: 6893-6899) as a template, to generate a fragment that can be ligated into the Δ24 shuttle vector. Once this new vector has been confirmed the cDNA of either mature hALB or mature mALB is cloned into the NheI site as described in Examples 6 & 8.

Generation of recombinant Ad vectors. A ClaI digested plasmid, pVK503 containing the RGD fiber, is used to allow for recombination of the E1/pIX region from the newly created pCX1-Δ24-pIX into the genome. This allows the generated AdΔ24S-RGD to have an analogous backbone to parental AdΔ24-RGD (Suzuki et al. (2001) Clin Cancer Res 7: 120-126). The resultant recombinant Ad genomes are checked with PCR methods and once confirmed, digested with PacI to release the viral genome and used to transfect 293 cells in order to rescue the appropriate adenovirus. Standard methods for propagation are undertaken in parallel on 293 cells and A549 cells, which have been previously used to propagate rescued AdΔ24-RGD (Suzuki et al. (2001) Clin Cancer Res 7: 120-126), followed by CsCl purification of virions. For all vectors standard OD₂₆₀ of DNA is used to determine viral particles units/ml (pu/ml). Infectivity is determined using the following fluorescent focus assay, which calculates focus forming units (ffu) (as described in Example 8), to determine the viral preparation quality (or viral particle to infectivity ratio (pu/ffu)). The presence of modified pIX is ascertained as described in Example 8.

Functionality of shielded AdΔ24S-RGD compared to AdΔ24-RGD. Viral replication and efficacy of the newly generated shielded AdΔ24S-RGD vector is confirmed with the parental AdΔ24-RGD vector through the following three methods: (i) cytopathic analysis (CPE), (ii) cytotoxicity analysis, and (iii) viral titer assessment, on a panel of cell lines known to allow replication of the parental AdΔ24-RGD. This panel of cell lines represent clinically relevant tissue types, in particular glioblastoma cell lines U-87MG, D-54MG, and T98G, ovarian cell lines SKOV3, and OVCAR3, and cervical cancer cell lines, C33A and HeLa as well as standard cell lines used for oncolytic Ad vector analysis, A549 and 293 cells. In addition, normal human astrocytes (NHA) (available from Clonetics Biowhittaker) are used under serum-starved conditions to represent an in vivo phenotype that is not permissible to AdΔ24-RGD replication (Fueyo et al. (2003) J Natl Cancer Inst 95: 652-660). The following control vectors, Ad.Luc.RGD, Ad300 wt, AdΔ24 and AdΔ24-RGD are used to compare with the newly generated AdΔ24S-RGD. For CPE analysis, vectors are seeded onto cells using an increasing dose of multiplicity of infection (MOI) from 0.001 to 100 pu/cell, and monitored over a 7-10 day period. At the end of this period, remaining cells are fixed and stained with crystal violet solution to allow for visual analysis of CPE. In the second analysis the set-up is repeated as for CPE, but after 7-10 days cell survival is determined using WST-1 (Sigma) staining. The number of living cells are calculated from noninfected cells cultured and treated with WST-1 in the same way as the experimental groups. Finally to determine viral progeny production, and hence titer, vectors are used at MOI 1 and following 48-96 hours, cells are harvested, freeze-thawed and viral progeny titered on 293 cells using the fluorescent focus assay. This allows for basic analysis of the efficacy of the AdΔ24S-RGD.

Experiment 2: Characterization of immune evasion shielded CRAd in vitro models.

Antibody evasion analysis of pIX-modified Ad vectors using monolayer cultures. Mouse sera from immunized C57BL/6 mice is used as a source of neutralizing antibodies. As a control mouse sera from naïve C57BL/6 mice is used, described as pre-immunized sera and post-immunized sera is obtained from C57BL/6 mice 14 days after immunization with Ad5 serotype virus. The vectors are pre-incubated in pre- and post-immunized mouse sera for 30 minutes at room temperature. In addition the experiment is performed using serially diluted sera as well as serum albumin. A smaller panel of cells are infected, using a smaller range of MOI (based on the findings from experiment 1). The same three analyses as in specific aim 1, CPE, cytotoxicity and viral titer attainment are performed.

Antibody evasion analysis of pIX-modified Ad vectors using spheroid model. To assess the ability of AdΔ24S-RGD to evade antibodies and replicate in a self-sustaining manner, spheroids are infected with shielded CRAds pre-exposed or un-exposed to mice sera containing A5 antibodies (as described in the previous experiment). This system has an advantage over monolayer culture and even raft cultures in that spheroids can be maintained up to 16 weeks (Kaaijk et al. (1995) Neuropathol Appl Neurobiol 21: 386-391) and thus viral replication assessed over a longer time period than in monolayer cultures. Spheroids of established glioblastoma cell lines, and ovarian cell lines are used for this experiment, unlike previous reports where fresh tumor tissue is used to establish the spheroids. Cells are cultured in 2% agarose-coated 48-well plates, in standard media conditions and after confirming viability by morphology spheroids of similar diameter (300-400 μm) are used for assessment of oncolytic activity of the experimental vector. Spheroids can be harvested at various time-points, and probed for various viral protein components, in particular the hexon protein on paraffin-embedded sections. Goat-anti-Ad hexon antibody (clone 1056, Chemicon) are used in immunohistochemical staining methodology and sections counterstained with hematoxylin.

Experiment 3: Evaluation of AdΔ24S-RGD performance in the Syrian Hamster Model.

In alternate embodiments the shielding protein could be a smaller domain of albumin, such domain one of albumin, or an alternate protein such as but not limited to myoglobin, alpha-1-antitrypsin or annexin V.

Furthermore, the shielding technology could be applied to any array of CRAds, such as those using tumour/tissue specific promoter control over the EIA region.

### Example 10

This Example provides plasmid maps and sequences of some of the preferred embodiments of the present invention.

FIGS. 10A and 10B depict the plasmid map and sequence of pSILucIXNhe, which is the starting plasmid for cloning shielding proteins next to the pIX gene. The NheI restriction site 3′ of pIX allows for insertion of cDNA of the shielding protein.

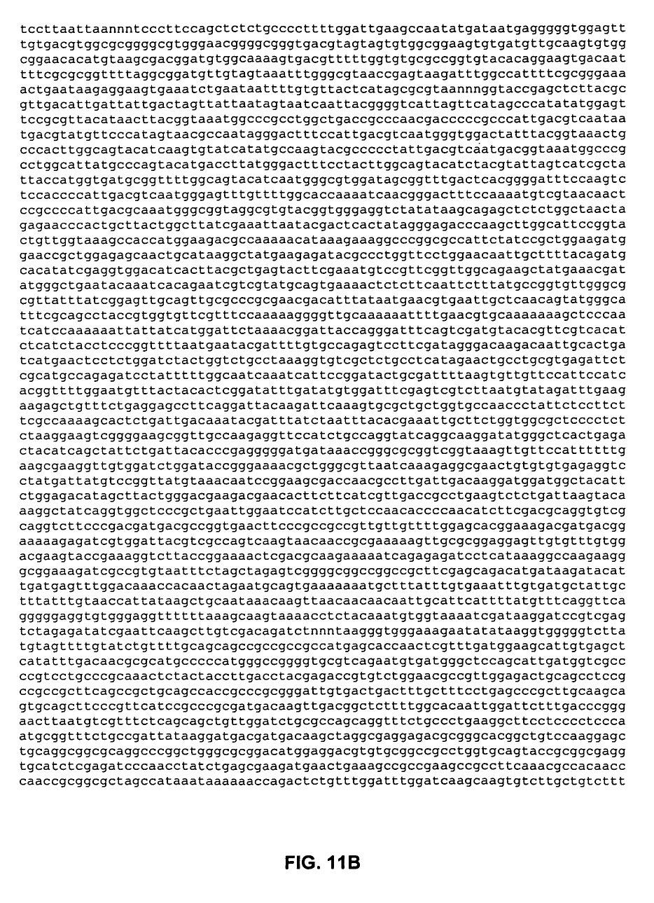

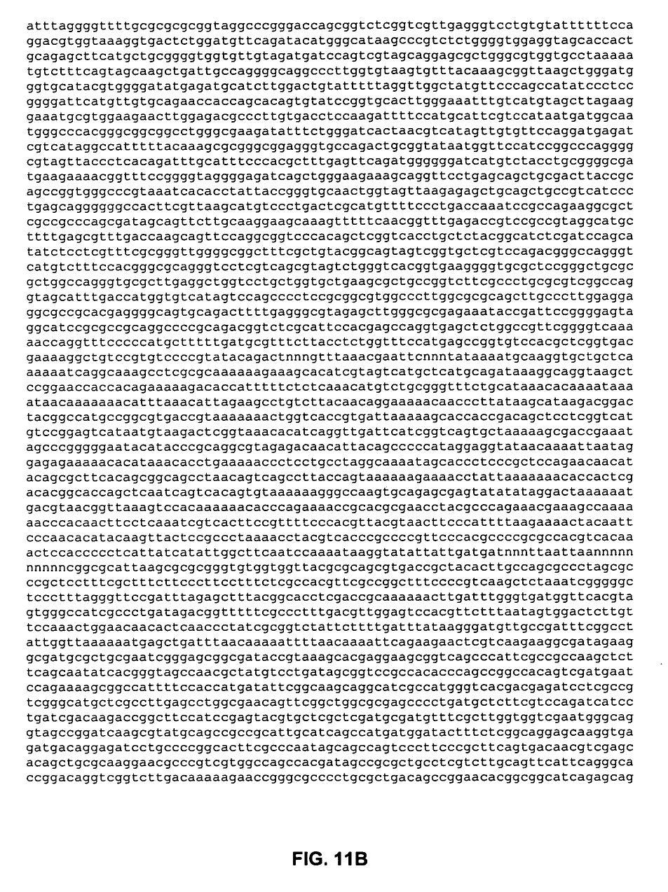

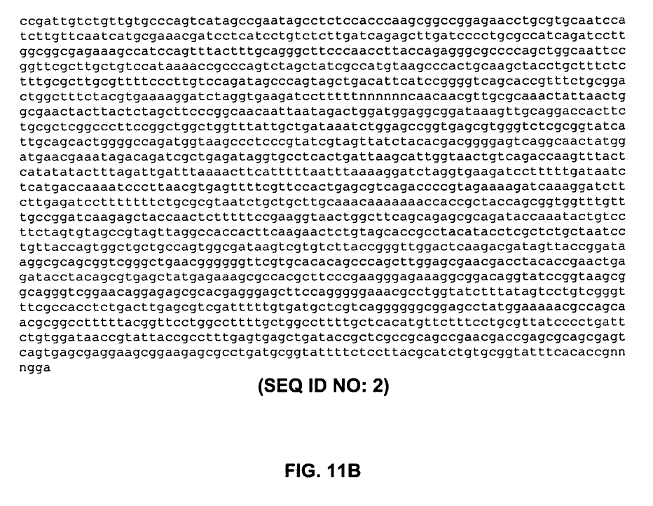

FIGS. 11A and 11B depict the plasmid map and sequence of pSILucIX-75A-NheI, which is the starting plasmid for cloning shielding proteins with a spacer peptide in between the pIX and shielding protein. This plasmid was derived from pSILucIXNhe by inserting a 75A spacer cDNA into the NheI restriction site 3′ of pIX. The 75A spacer consists of aas and is based on the 75A spacer described by Velling a et al, 2004. This 75A spacer was created by PCR methodologies and the cDNA was then digested with AvrII (to create the 5′ ligation end—AvrII has a compatible overhang with NheI restriction site) and NheI (to create the 3′ ligation end). This allows insertion of the cDNA into the NheI restriction site and maintains the unique NheI restriction site for cloning of shielding proteins into the plasmid.

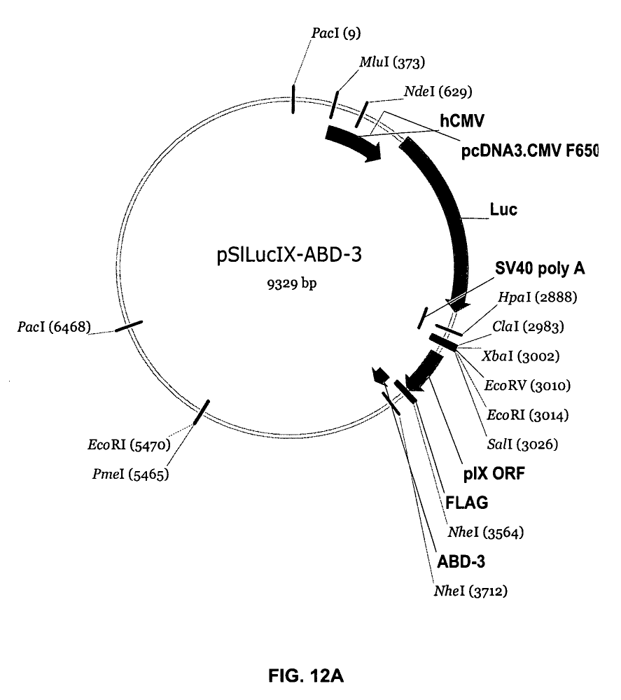

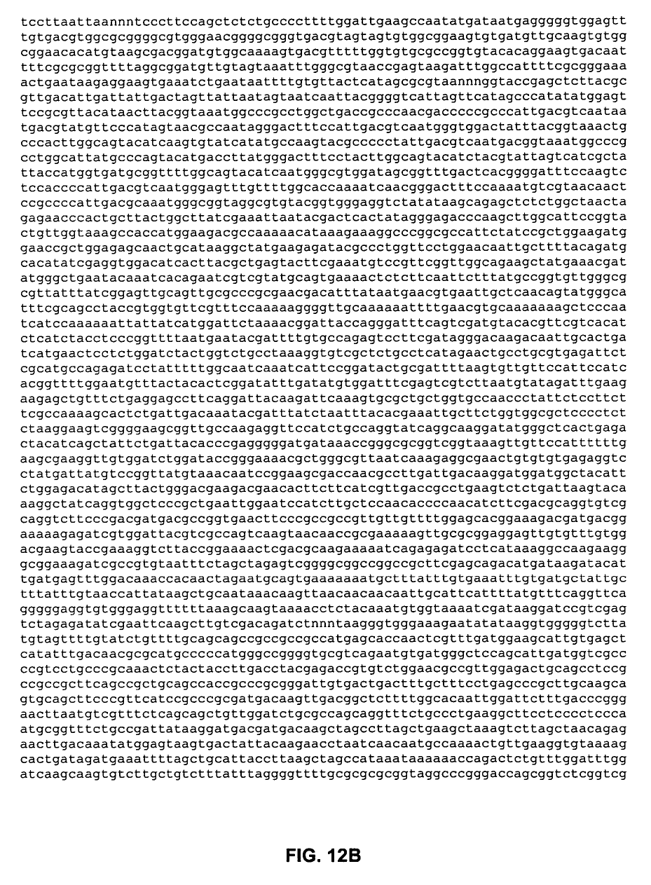

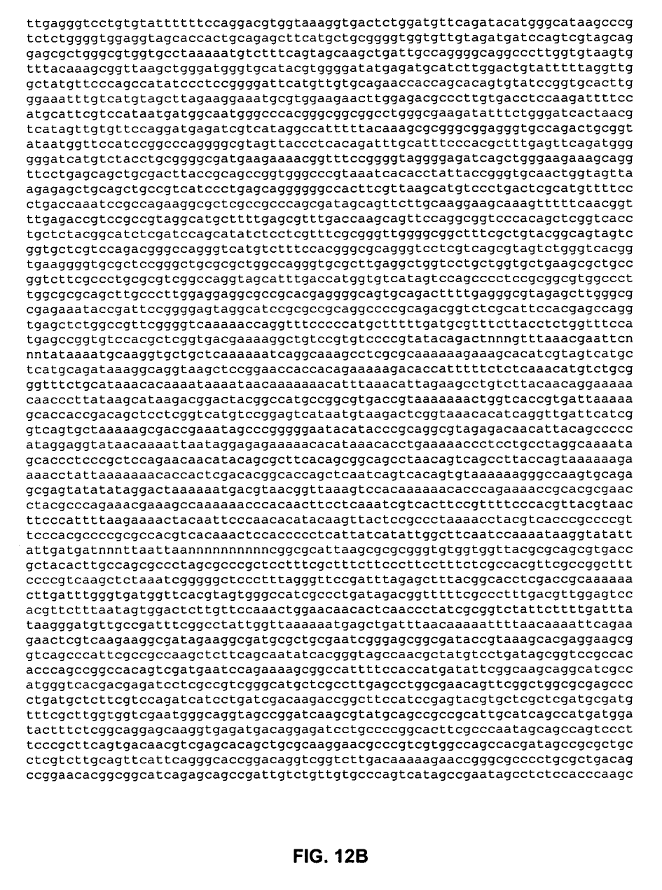

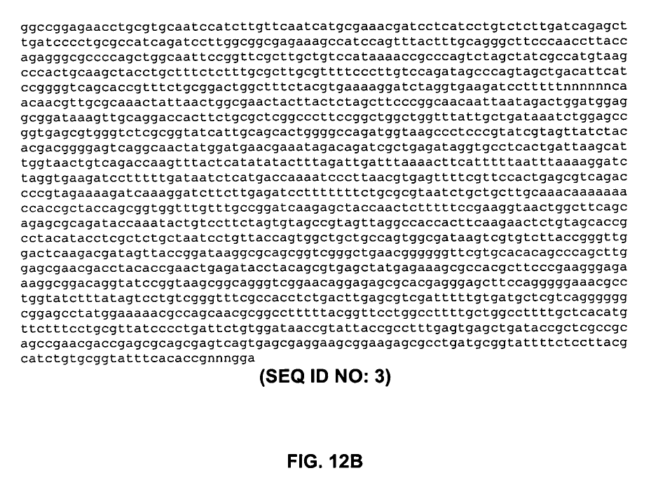

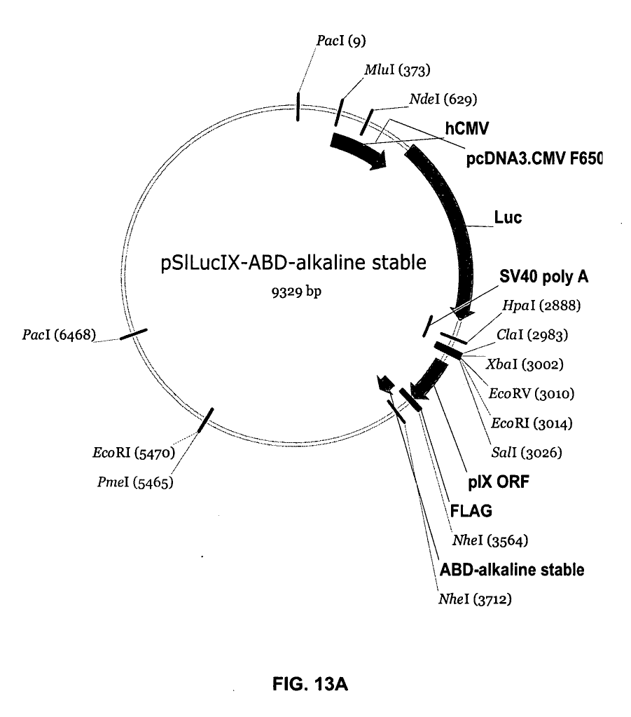

FIGS. 12A and 12B depict the plasmid map and sequence of pSILucIX-ABD-3, which contains the albumin binding domain, ABD-3 from streptococcal protein G, fused to pIX. ABD-3 consists of 46aas and was been cloned into the NheI restriction site of pSILucIXNhe. The cDNA for ABD-3 was generated by annealing two single stranded oligos, each with a 15 nucleotide compatible overlap and were extended with Taq polymerase. The double stranded generated fragment was then digested with NheI and ligated into NheI digested pSILucIXNhe. To generate the Ad genome with the modified pIX, this plasmid was PmeI digested and recombined with pAdEasy, and the recombinant genomes were PacI digested to allow for rescue of virus in 293 cells.

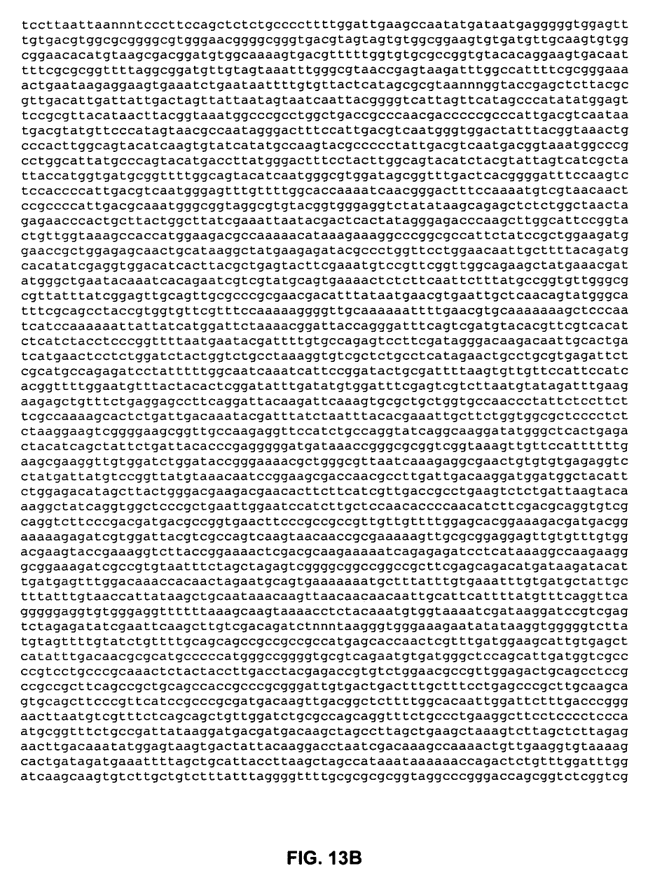

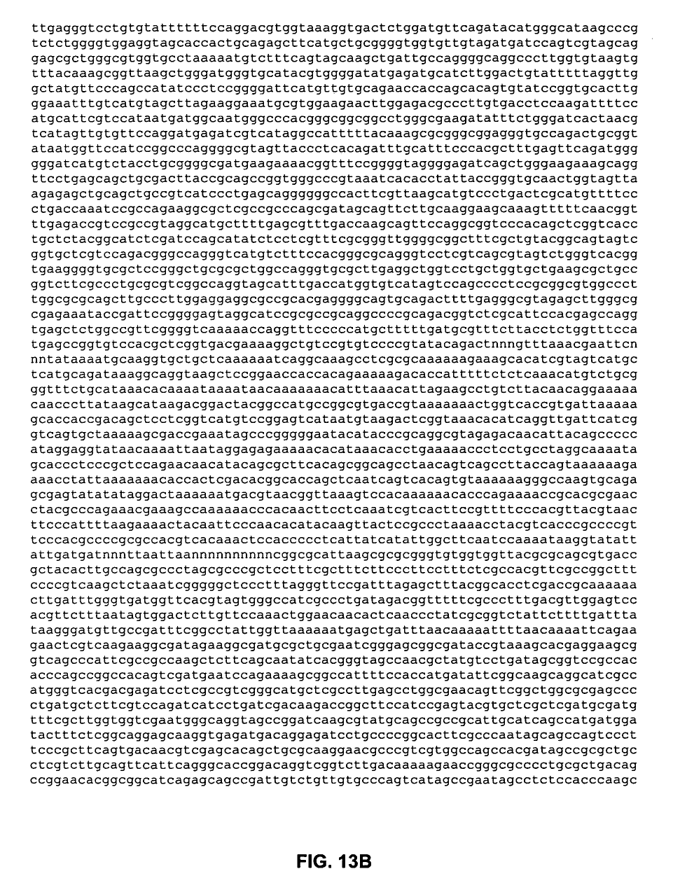

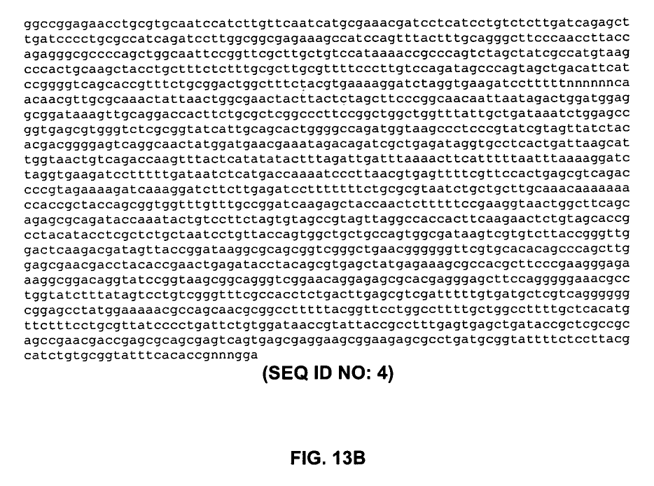

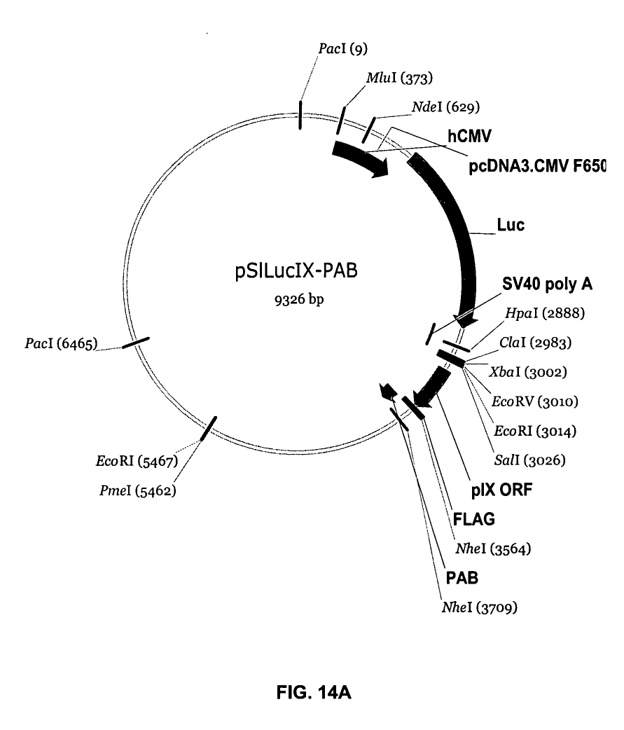

FIGS. 13A and 13B depict the plasmid map and sequence of pSILucIX-ABD-AS. This plasmid contains a modified, alkaline stable form of ABD-3 fused to pIX as described by (Gulich et al. Protein Engineering 2002, 15: 835-842). ABD-AS of 46aas and was been cloned into the NheI restriction site of pSILucIXNhe. The cDNA for ABD-AS was generated by annealing two single stranded oligos, each with a 15 nucleotide compatible overlap and were extended with Taq polymerase. The double stranded generated fragment was then digested with NheI and ligated into NheI digested pSILucIXNhe. To generate the Ad genome with the modified pIX, this plasmid was PmeI digested and recombined with pAdEasy, and the recombinant genomes were PacI digested to allow for rescue of virus in 293 cells.

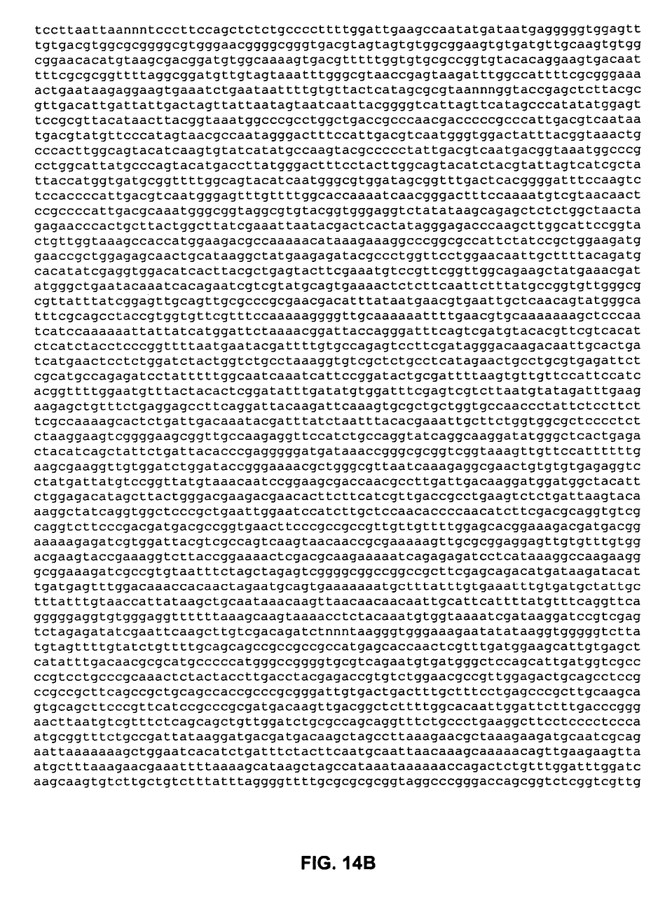

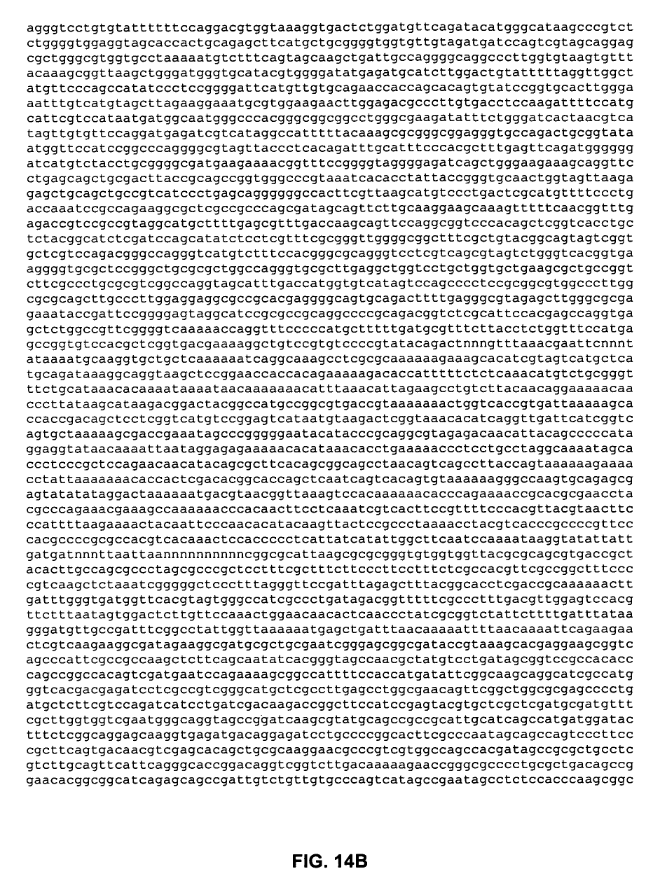

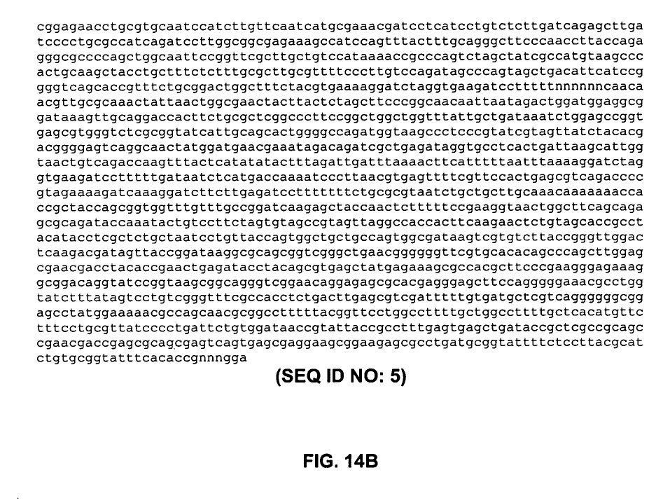

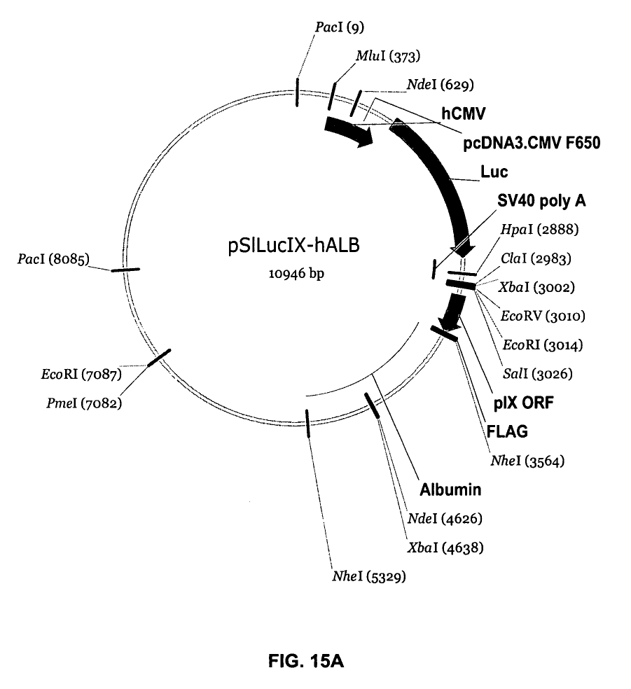

FIGS. 14A and 14B depict the plasmid map and sequence of pSILucIX-PAB, which contains the albumin binding domain, ALB8, termed here as PAB, from the PAB protein of Peptostreptococcus magnus bacteria. PAB consists of the consensus albumin binding sequence of 45aas sequence described by (Johansson et al. J Mol Biol 2002, 316: 1083-1099) and was been cloned into the NheI restriction site of pSILucIXNhe. The cDNA for PAB was generated by annealing two single stranded oligos, each with a 15 nucleotide compatible overlap and were extended with Taq polymerase. The double stranded generated fragment was then digested with NheI and ligated into NheI digested pSILucIXNhe. To generate the Ad genome with the modified pIX, this plasmid was PmeI digested and recombined with pAdEasy, and the recombinant genomes were PacI digested to allow for rescue of virus in 293 cells.

FIGS. 15A and 15B depict the plasmid map and sequence of pSILucIX-hALB, which contains the cDNA of human albumin cloned into the NheI site of pSILucIXNheI. The cDNA was generated by PCR, using primers with NheI restrictions sites present, of the commercially available Origene plasmid, TC125510 (Acc No. NM_{—}000477). The generated fragment was digested with NheI for cloning. To generate the Ad genome with the modified pIX, this plasmid was PmeI digested and recombined with pAdEasy, and the recombinant genomes were PacI digested to allow for rescue of virus in 293 cells.

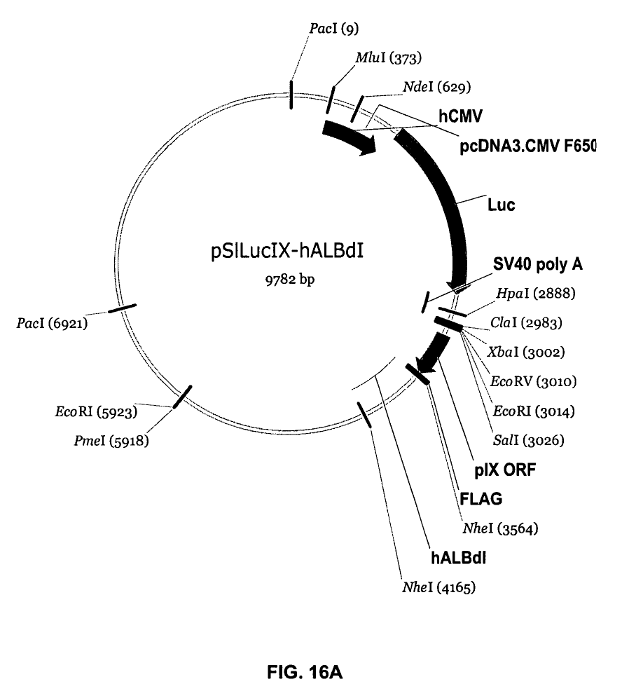

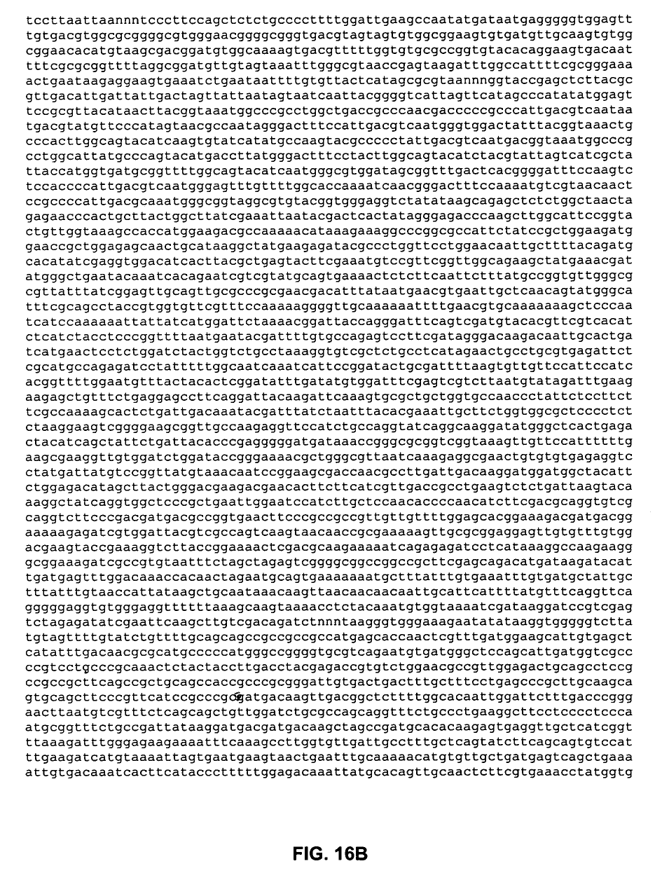

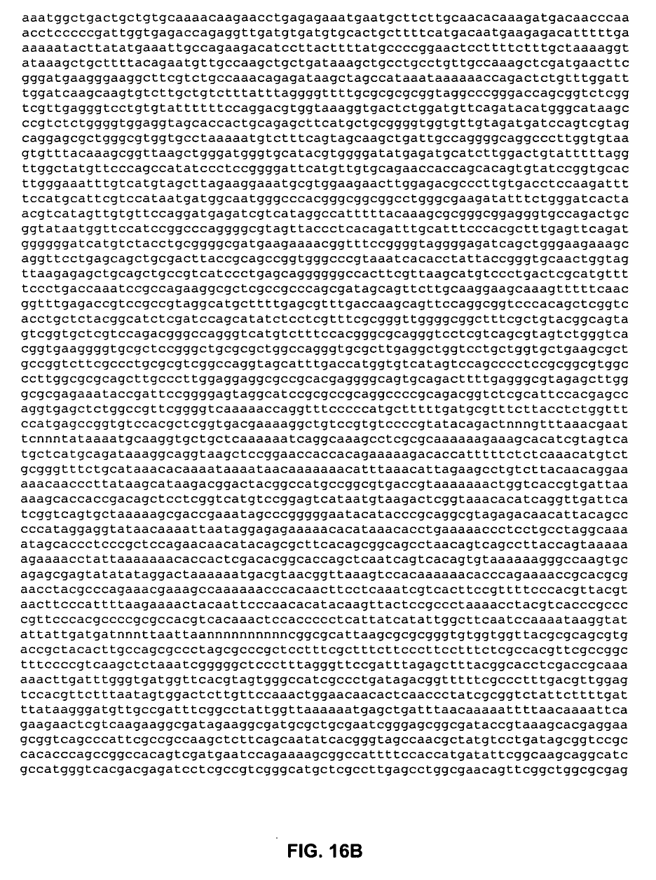

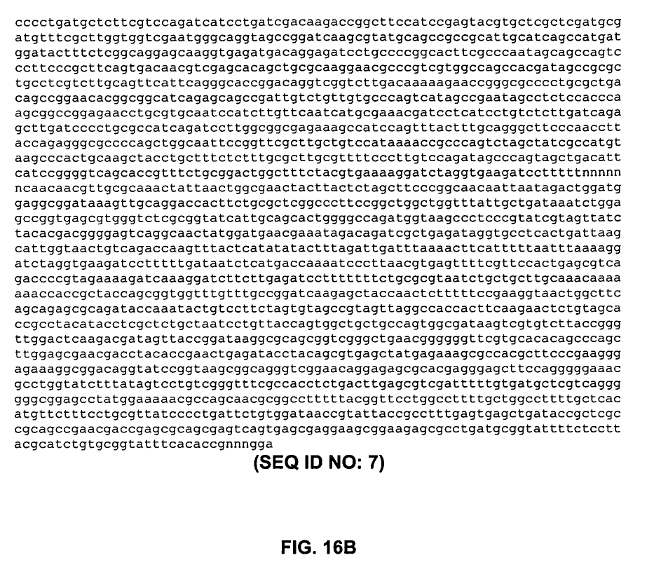

FIGS. 16A and 16B depict the plasmid map and sequence of pSILucIX-hALBdI, which plasmid contains domain I of human albumin cloned into the NheI site of pSILucIXNheI. Human albumin consists of three major domains, and the three domains have been delinearated as domain I amino acids 1-197, domain II amino acids 189-385 and domain III amino acids 381-585 (Dockal et al. J Biol Chem 1999, 274: 29303-29310). The cDNA was generated by PCR, using primers with NheI restrictions sites present, of the commercially available Origene plasmid TC125510 (Acc No. NM_{—}000477). The generated fragment was digested with NheI for cloning. To generate the Ad genome with the modified pIX, this plasmid was PmeI digested and recombined with pAdEasy, and the recombinant genomes were PacI digested to allow for rescue of virus in 293 cells.

FIGS. 17A and 17B depict the plasmid map and sequence of pSILucIX-ANXV, which contains annexin V cloned into the NheI site of pSILucIXNheI. The cDNA was generated by PCR, using primers with NheI restrictions sites present, of the commercially available Origene plasmid, TC128133 (Acc No. Nm-001154). The generated fragment was digested with NheI for cloning. To generate the Ad genome with the modified pIX, this plasmid was PmeI digested and recombined with pAdEasy, and the recombinant genomes were PacI digested to allow for rescue of virus in 293 cells.

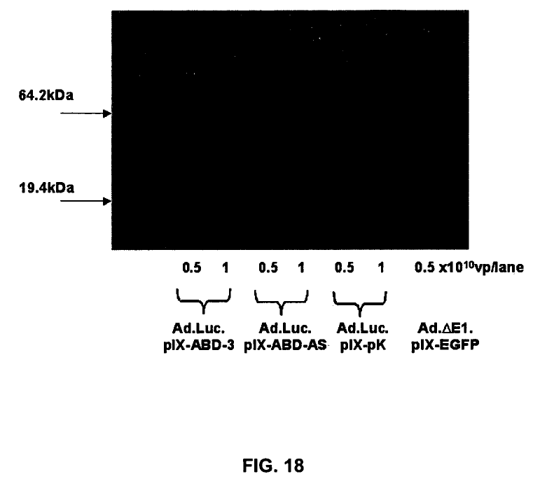

FIG. 18 depicts the incorporation of pIX-ABD into virions. Viruses were rescued from 293 cells transfected with the Ad genomes containing pIX-ABD-3 or pIX-ABD-AS, Ad.Luc.pIX-ABD-3 and Ad.Luc.pIX-ABD-AS (as described in FIGS. 3 and 4). These viruses were propagated and purified by standard CsCl gradients. Purified virions were denatured at 96° C. in laemmli buffer and 0.5 and 1×10¹⁰ pu were loaded to SDS-PAGE gel for protein separation. Proteins were then transferred to a membrane and probed with an anti-Flag antibody (the pIX constructs contain a Flag tag nucleotide sequence) and developed with WesternBreeze kit. In addition to the ABD viruses, Ad.Luc.IX-pK (Dmitriev et al. J Virol 2002, 76:6893-6899) and Ad.ΔE1.pIX-EGFP (Le et al. Mol Imaging 2005, 3: 105-116) were run as controls for the anti-Flag antibody. The modified pIX-ABD proteins migrate to approximately 19.4 kDa and the image demonstrates that pIX-ABD-3 and pIX-ABD-AS are present in the virion capsids.

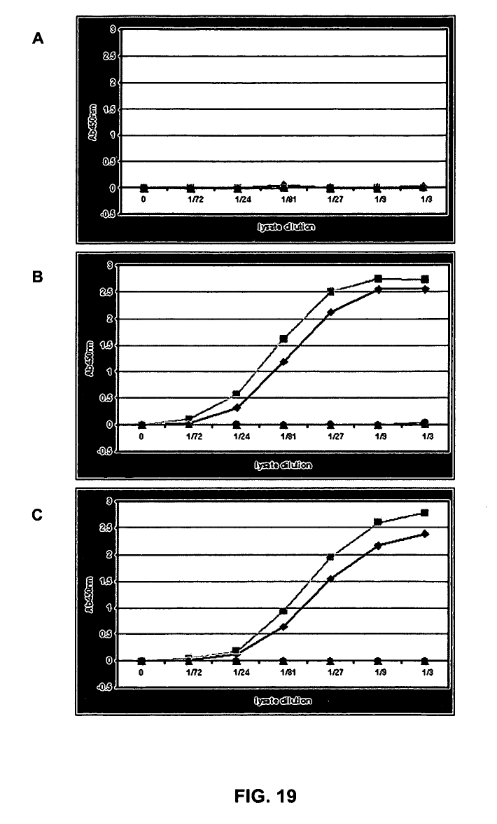

FIGS. 19A-C depicts the detection of human and mouse albumin by pIX-ABD-3 and pIX-ABD-AS fusion proteins. CsCl purified virions of Ad.Luc.1, containing wild type pIX virus, Ad.Luc.IX-ABD-3, Ad.Luc.IX-ABD-AS were used to infect 293 cells at 100 viral particles per cell. After 3 days cells were harvested and freeze-thawed and the lysates centrifuge to remove cellular debris. These lysates were then applied to ELISA plates adsorbed with human, murine or bovine albumin, or just plastic and analyzed the functionality of albumin binding domains within the context of pIX. FIG. 19A (top panel), FIG. 19B (middle panel) and FIG. 19C (bottom panel) demonstrate the results for Ad.Luc.1, Ad.Luc.IX-ABD-3 and Ad.Luc.IX-ABD-AS for binding to human albumin (diamonds), mouse albumin (squares), bovine albumin (circles) and plastic (triangles). Ad.Luc. 1 does not bind to any of the albumins nor plastic while both ABD viruses bind to human and mouse albumin but not bovine nor plastic. Therefore the ABD domains fused to pIX retain their functionality within the capsid protein-incorporated context.

The invention is further described by the following numbered paragraphs:

1. A chimeric pIX protein having at least an adenoviral pIX peptide sequence and a non-native amino acid sequence encoding a protein that interferes with adenovirus specific antibody binding to an adenovirus capsid, wherein the non-native amino acid constitutes the C-terminus of the chimeric protein.

2. The chimeric pIX protein of paragraph 1, wherein the non-native amino acid sequence is a self protein.

3. The chimeric pIX protein of paragraph 1, wherein the non-native amino acid sequence is a serum protein, an albumin related protein or an alpha 1 antitripsin related protein.

4. The chimeric pIX protein of paragraph 1, wherein the non-native amino acid sequence is a single chain antibody.

5. The chimeric pIX protein of paragraph 1, wherein the non-native amino acid sequence is a ligand that binds to a substrate present on the surface of a cell.

6. A nucleic acid encoding the chimeric pIX protein of paragraph 1.

7. An adenoviral capsid containing a chimeric pIX protein having at least an adenoviral pIX peptide sequence and a non-native amino acid sequence encoding a protein that interferes with adenovirus specific antibody binding to the adenovirus capsid, wherein the non-native amino acid sequence constitutes the C-terminus of the chimeric protein.

8. The adenoviral capsid of paragraph 7, wherein the adenovirus specific antibody binding to the adenovirus capsid is reduced by about 50%.

9. The adenoviral capsid of paragraph 7, wherein the non-native amino acid is a self protein.

10. The adenoviral capsid of paragraph 7, wherein the non-native amino acid sequence is a serum protein, an albumin related protein or an alpha 1 antitripsin related protein.

11. The adenoviral capsid of paragraph 7, wherein the non-native amino acid sequence is a single chain antibody.

12. The adenoviral capsid of paragraph 7, comprising a mutant adenoviral fiber protein having an affinity for a native adenoviral cellular receptor of at least about an order of magnitude less than a wild-type adenoviral fiber protein.

13. The adenoviral capsid of paragraph 7, comprising an adenoviral hexon protein having a mutation affecting at least one native HVR sequence.

14. The adenoviral capsid of paragraph 7, lacking a native glycosylation or phosphorylation site.

15. The adenoviral capsid of paragraph 7, which elicits less immunogenicity in a host animal than does a wild-type adenovirus.

16. The adenoviral capsid of paragraph 7, comprising a second non-adenoviral ligand conjugated to a fiber, a penton, a hexon, a protein IIIa or a protein VI.

17. A composition comprising the adenoviral capsid of paragraph 7 and a nucleic acid.

18. An adenoviral vector comprising the adenoviral capsid of paragraph 7 and an adenoviral genome.

19. The adenoviral capsid of paragraph 16, wherein the non-native amino acid is a ligand and wherein the second non-adenoviral ligand recognizes the same substrate as the non-native amino acid.

20. The adenoviral vector of paragraph 18, which is replication incompetent.

21. The adenoviral vector of paragraph 18, which does not productively infect HEK-293 cells.

22. The adenoviral vector of paragraph 18, wherein the adenoviral genome comprises a non-native nucleic acid.

23. The adenoviral vector of paragraph 18, which is replication competent.

24. The adenoviral vector of paragraph 18, wherein the adenoviral genome comprises a non-native nucleic acid.

25. A method of infecting a cell, comprising contacting a cell with an adenoviral vector of paragraph 18.

26. The adenoviral vector of paragraph 24, wherein the non-native nucleic acid for transcription is operably linked to a non-adenoviral promoter.

27. The adenoviral vector of paragraph 26, wherein the non-adenoviral promoter is a cell or tissue-specific promoter.

28. The adenoviral vector of paragraph 24, wherein the non-adenoviral promoter is a regulable promoter.

29. The adenoviral vector of paragraph 24, wherein the non-native nucleic acid for transcription is operably linked to an adenoviral promoter.

30. A method for administering viral vectors to a mammal, said method comprising the steps of:

(a) contacting a host cell with a chimeric pIX-modified recombinant virus according to paragraph 7; and

(b) contacting the mammal with a recombinant virus.

31. The method according to paragraph 30, wherein the recombinant virus of (b) comprises a second chimeric pIX-modified recombinant virus.

32. The method of paragraph 30, wherein the mammal is a human.

33. A method for shielding an adenoviral vector from a humoral response comprising incorporating a protein into an adenoviral capsid.

34. The method of paragraph 33 wherein the protein is a serum protein, albumin, alpha-1-antitrypsin, an antibody or a self protein.

35. The method of paragraph 33 wherein protein is protein A of Staphylococcus aureas, protein G of group C and G streptococci or protein PAB from Peptostreptococcus magnus.

36. The method of paragraph 33 wherein the protein is a Zc-binding domain of Staphylococcus aureus protein A.

37. The method of any one of paragraphs 33 to 36 wherein the protein is incorporated into the fiber, hexon, penton base, pIX or pIII of the capsid or combinations thereof.

38. The method of any one of paragraphs 33 to 37 wherein the vector is replication incompetent.

39. The method of any one of paragraphs 33 to 37 wherein the vector is replication competent.

40. The method of any one of paragraphs 33 to 39 further comprising incubating the adenoviral vector in vitro with shielding moieties.

41. The method of paragraph 40 wherein the shielding moieties are human serum proteins, albumin or antibodies.

42. The method of paragraph 40 wherein the shielding moieties are self proteins of a mammal.

43. A method for administering a shielded adenoviral vector to a mammal in need thereof comprising administering a therapeutically effective amount of the vector of any one of paragraphs 33 to 42, wherein the vector further comprises a targeting ligand, to the mammal wherein the targeting ligand binds to a target cell such that the adenovirus infects the target cell.

44. The method of paragraph 43 wherein the vector is administered in multiple doses.

45. The adenoviral AdΔ24S-RGD comprising the capsid of any one of paragraphs 7-44.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A chimeric pIX protein having at least an adenoviral pIX peptide sequence and a non-native amino acid sequence encoding a protein that interferes with adenovirus specific antibody binding to an adenovirus capsid, wherein the non-native amino acid constitutes the C-terminus of the chimeric protein.

2. The chimeric pIX protein of claim 1,
wherein the non-native amino acid sequence is a self protein or
wherein the non-native amino acid sequence is a serum protein, an albumin related protein or an alpha 1 antitripsin related protein or
wherein the non-native amino acid sequence is a single chain antibody or
wherein the non-native amino acid sequence is a ligand that binds to a substrate present on the surface of a cell.

3. A nucleic acid encoding the chimeric pIX protein of claim 1.

4. An adenoviral capsid containing a chimeric pIX protein having at least an adenoviral pIX peptide sequence and a non-native amino acid sequence encoding a protein that interferes with adenovirus specific antibody binding to the adenovirus capsid, wherein the non-native amino acid sequence constitutes the C-terminus of the chimeric protein.

5. The adenoviral capsid of claim 4,
wherein the adenovirus specific antibody binding to the adenovirus capsid is reduced by about 50% or
wherein the non-native amino acid is a self protein or
wherein the non-native amino acid sequence is a serum protein, an albumin related protein or an alpha 1 antitripsin related protein or
wherein the non-native amino acid sequence is a single chain antibody or
comprising a mutant adenoviral fiber protein having an affinity for a native adenoviral cellular receptor of at least about an order of magnitude less than a wild-type adenoviral fiber protein or
comprising an adenoviral hexon protein having a mutation affecting at least one native HVR sequence or
lacking a native glycosylation or phosphorylation site or
which elicits less immunogenicity in a host animal than does a wild-type adenovirus or
comprising a second non-adenoviral ligand conjugated to a fiber, a penton, a hexon, a protein IIIa or a protein VI.

6. A composition comprising the adenoviral capsid of claim 4 and a nucleic acid.

7. An adenoviral vector comprising the adenoviral capsid of claim 4 and an adenoviral genome.

8. The adenoviral capsid of claim 4, comprising a second non-adenoviral ligand conjugated to a fiber, a penton, a hexon, a protein IIIa or a protein VI and wherein the non-native amino acid is a ligand and wherein the second non-adenoviral ligand recognizes the same substrate as the non-native amino acid.

9. The adenoviral vector of claim 7,
which is replication incompetent or
which does not productively infect HEK-293 cells or
wherein the adenoviral genome comprises a non-native nucleic acid or
which is replication competent or
wherein the adenoviral genome comprises a non-native nucleic acid.

10. A method of infecting a cell, comprising contacting a cell with an adenoviral vector of claim 7.

11. The adenoviral vector of claim 7, wherein the adenoviral genome comprises a non-native nucleic acid and
wherein the non-native nucleic acid for transcription is operably linked to a non-adenoviral promoter or
wherein the non-adenoviral promoter is a regulable promoter or
wherein the non-native nucleic acid for transcription is operably linked to an adenoviral promoter.

12. The adenoviral vector of claim 11, wherein the non-adenoviral promoter is a cell or tissue-specific promoter.

13. A method for administering viral vectors to a mammal, said method comprising the steps of:
(a) contacting a host cell with a chimeric pIX-modified recombinant virus according to claim 4; and
(b) contacting the mammal with a recombinant virus.

14. The method according to claim 13, wherein the recombinant virus of (b) comprises a second chimeric pIX-modified recombinant virus or
wherein the mammal is a human.

15. A method for shielding an adenoviral vector from a humoral response comprising incorporating a protein into an adenoviral capsid.

16. The method of claim 15,
wherein the protein is a serum protein, albumin, alpha-1-antitrypsin, an antibody or a self protein or
wherein protein is protein A of Staphylococcus aureas, protein G of group C and G streptococci or protein PAB from Peptostreptococcus magnus or
wherein the protein is a Zc-binding domain of Staphylococcus aureus protein A or
wherein the protein is incorporated into the fiber, hexon, penton base, pIX or pIII of the capsid or combinations thereof or
wherein the vector is replication incompetent or
wherein the vector is replication competent.

17. The method of claim 15 further comprising incubating the adenoviral vector in vitro with shielding moieties.

18. The method of claim 17
wherein the shielding moieties are human serum proteins, albumin or antibodies or
wherein the shielding moieties are self proteins of a mammal.

19. A method for administering a shielded adenoviral vector to a mammal in need thereof comprising administering a therapeutically effective amount of the vector of claim 15, wherein the vector further comprises a targeting ligand, to the mammal wherein the targeting ligand binds to a target cell such that the adenovirus infects the target cell.

20. The method of claim 19 wherein the vector is administered in multiple doses.


SEQUENCE LISTING (9 sequences)

SEQ ID NO: 1 - DNA, 9274 bp, Artificial Sequence
  Description of Artificial Sequence Synthetic nucleotide sequence
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactggtaccgagctcttacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatttctagctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgagtctagagatatcgaattcaagcttgtcgactcgaagatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttctgccgattataaggatgacgatgacaagctagccataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagagggagtttaaacgaattcaatagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaacatgcatggatccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagccatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattttgttaaaatttttgttaaatcagctcattttttaaccaataggccgaaatcggcaacatcccttataaatcaaaagaatagaccgcgatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgcgcgcttaatgcgccgctacagggcgcgtccattcgccattcaggatcgaattaattcttaattaa

SEQ ID NO: 2 - DNA, 9379 bp, Artificial Sequence
  Description of Artificial Sequence Synthetic nucleotide sequence
  modified_base (12)..(14): a, c, g, t, unknown or other
  modified_base (355)..(357): a, c, g, t, unknown or other
  modified_base (3037)..(3039): a, c, g, t, unknown or other
  modified_base (5508)..(5510): a, c, g, t, unknown or other
  modified_base (5525)..(5527): a, c, g, t, unknown or other
  modified_base (6510)..(6512): a, c, g, t, unknown or other
  modified_base (6521)..(6530): a, c, g, t, unknown or other
  modified_base (8068)..(8073): a, c, g, t, unknown or other
  modified_base (9374)..(9376): a, c, g, t, unknown or other
tccttaattaannntcccttccagctctctgccccttttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaannnggtaccgagctcttacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatttctagctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgagtctagagatatcgaattcaagcttgtcgacagatctnnntaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttctgccgattataaggatgacgatgacaagctaggcgaggagacgcgggcacggctgtccaaggagctgcaggcggcgcaggcccggctgggcgcggacatggaggacgtgtgcggccgcctggtgcagtaccgcggcgaggtgcatctcgagatcccaacctatctgagcgaagatgaactgaaagccgccgaagccgccttcaaacgccacaacccaaccgcggcgctagccataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagactnnngtttaaacgaattcnnntataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatnnnttaattaannnnnnnnnncggcgcattaagcgcgcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatcgcggtctattcttttgatttataagggatgttgccgatttcggcctattggttaaaaaatgagctgatttaacaaaaattttaacaaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttnnnnnncaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgnnngga

SEQ ID NO: 3 - DNA, 9329 bp, Artificial Sequence
  Description of Artificial Sequence Synthetic nucleotide sequence
  modified_base (12)..(14): a, c, g, t, unknown or other
  modified_base (355)..(357): a, c, g, t, unknown or other
  modified_base (3037)..(3039): a, c, g, t, unknown or other
  modified_base (5458)..(5460): a, c, g, t, unknown or other
  modified_base (5475)..(5477): a, c, g, t, unknown or other
  modified_base (6460)..(6462): a, c, g, t, unknown or other
  modified_base (6471)..(6480): a, c, g, t, unknown or other
  modified_base (8018)..(8023): a, c, g, t, unknown or other
  modified_base (9324)..(9326): a, c, g, t, unknown or other
tccttaattaannntcccttccagctctctgccccttttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaannnggtaccgagctcttacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatttctagctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgagtctagagatatcgaattcaagcttgtcgacagatctnnntaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttctgccgattataaggatgacgatgacaagctagccttagctgaagctaaagtcttagctaacagagaacttgacaaatatggagtaagtgactattacaagaacctaatcaacaatgccaaaactgttgaaggtgtaaaagcactgatagatgaaattttagctgcattaccttaagctagccataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagactnnngtttaaacgaattcnnntataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatnnnttaattaannnnnnnnnncggcgcattaagcgcgcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatcgcggtctattcttttgatttataagggatgttgccgatttcggcctattggttaaaaaatgagctgatttaacaaaaattttaacaaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttnnnnnncaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgnnngga

SEQ ID NO: 4 - DNA, 9329 bp, Artificial Sequence
  Description of Artificial Sequence Synthetic nucleotide sequence
  modified_base (12)..(14): a, c, g, t, unknown or other
  modified_base (355)..(357): a, c, g, t, unknown or other
  modified_base (3037)..(3039): a, c, g, t, unknown or other
  modified_base (5458)..(5460): a, c, g, t, unknown or other
  modified_base (5475)..(5477): a, c, g, t, unknown or other
  modified_base (6460)..(6462): a, c, g, t, unknown or other
  modified_base (6471)..(6480): a, c, g, t, unknown or other
  modified_base (8018)..(8023): a, c, g, t, unknown or other
  modified_base (9324)..(9326): a, c, g, t, unknown or other
tccttaattaannntcccttccagctctctgccccttttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaannnggtaccgagctcttacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatttctagctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgagtctagagatatcgaattcaagcttgtcgacagatctnnntaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttctgccgattataaggatgacgatgacaagctagccttagctgaagctaaagtcttagctcttagagaacttgacaaatatggagtaagtgactattacaaggacctaatcgacaaagccaaaactgttgaaggtgtaaaagcactgatagatgaaattttagctgcattaccttaagctagccataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagactnnngtttaaacgaattcnnntataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatnnnttaattaannnnnnnnnncggcgcattaagcgcgcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatcgcggtctattcttttgatttataagggatgttgccgatttcggcctattggttaaaaaatgagctgatttaacaaaaattttaacaaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttnnnnnncaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgnnngga

SEQ ID NO: 5 - DNA, 9326 bp, Artificial Sequence
  Description of Artificial Sequence Synthetic nucleotide sequence
  modified_base (12)..(14): a, c, g, t, unknown or other
  modified_base (355)..(357): a, c, g, t, unknown or other
  modified_base (3037)..(3039): a, c, g, t, unknown or other
  modified_base (5455)..(5457): a, c, g, t, unknown or other
  modified_base (5472)..(5474): a, c, g, t, unknown or other
  modified_base (6457)..(6459): a, c, g, t, unknown or other
  modified_base (6468)..(6477): a, c, g, t, unknown or other
  modified_base (8015)..(8020): a, c, g, t, unknown or other
  modified_base (9321)..(9323): a, c, g, t, unknown or other
tccttaattaannntcccttccagctctctgccccttttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaannnggtaccgagctcttacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatttctagctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgagtctagagatatcgaattcaagcttgtcgacagatctnnntaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttctgccgattataaggatgacgatgacaagctagccttaaagaacgctaaagaagatgcaatcgcagaattaaaaaaagctggaatcacatctgatttctacttcaatgcaattaacaaagcaaaaacagttgaagaagttaatgctttaaagaacgaaattttaaaagcataagctagccataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagactnnngtttaaacgaattcnnntataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatnnnttaattaannnnnnnnnncggcgcattaagcgcgcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatcgcggtctattcttttgatttataagggatgttgccgatttcggcctattggttaaaaaatgagctgatttaacaaaaattttaacaaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttnnnnnncaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgnnngga

SEQ ID NO: 6 - DNA, 10946 bp, Artificial Sequence
  Description of Artificial Sequence Synthetic nucleotide sequence
  modified_base (12)..(14): a, c, g, t, unknown or other
  modified_base (355)..(357): a, c, g, t, unknown or other
  modified_base (3037)..(3039): a, c, g, t, unknown or other
  modified_base (7075)..(7077): a, c, g, t, unknown or other
  modified_base (7092)..(7094): a, c, g, t, unknown or other
  modified_base (8077)..(8079): a, c, g, t, unknown or other
  modified_base (8088)..(8097): a, c, g, t, unknown or other
  modified_base (9635)..(9640): a, c, g, t, unknown or other
  modified_base (10941)..(10943): a, c, g, t, unknown or other
tccttaattaannntcccttccagctctctgccccttttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaannnggtaccgagctcttacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatttctagctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgagtctagagatatcgaattcaagcttgtcgacagatctnnntaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttctgccgattataaggatgacgatgacaagctagccgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataagctagccataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagactnnngtttaaacgaattcnnntataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatnnnttaattaannnnnnnnnncggcgcattaagcgcgcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatcgcggtctattcttttgatttataagggatgttgccgatttcggcctattggttaaaaaatgagctgatttaacaaaaattttaacaaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttnnnnnncaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgnnngga

SEQ ID NO: 7 - DNA, 9782 bp, Artificial Sequence
  Description of Artificial Sequence Synthetic nucleotide sequence
  modified_base (12)..(14): a, c, g, t, unknown or other
  modified_base (355)..(357): a, c, g, t, unknown or other
  modified_base (3037)..(3039): a, c, g, t, unknown or other
  modified_base (5911)..(5913): a, c, g, t, unknown or other
  modified_base (5928)..(5930): a, c, g, t, unknown or other
  modified_base (6913)..(6915): a, c, g, t, unknown or other
  modified_base (6924)..(6933): a, c, g, t, unknown or other
  modified_base (8471)..(8476): a, c, g, t, unknown or other
  modified_base (9777)..(9779): a, c, g, t, unknown or other
tccttaattaannntcccttccagctctctgccccttttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaannnggtaccgagctcttacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatttctagctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgagtctagagatatcgaattcaagcttgtcgacagatctnnntaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttctgccgattataaggatgacgatgacaagctagccgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagataagctagccataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagactnnngtttaaacgaattcnnntataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatnnnttaattaannnnnnnnnncggcgcattaagcgcgcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatcgcggtctattcttttgatttataagggatgttgccgatttcggcctattggttaaaaaatgagctgatttaacaaaaattttaacaaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttnnnnnncaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgnnngga

SEQ ID NO: 8 - DNA, 10148 bp, Artificial Sequence
  Description of Artificial Sequence Synthetic nucleotide sequence
  modified_base (12)..(14): a, c, g, t, unknown or other
  modified_base (355)..(357): a, c, g, t, unknown or other
  modified_base (3037)..(3039): a, c, g, t, unknown or other
  modified_base (6277)..(6279): a, c, g, t, unknown or other
  modified_base (6294)..(6296): a, c, g, t, unknown or other
  modified_base (7279)..(7281): a, c, g, t, unknown or other
  modified_base (7290)..(7299): a, c, g, t, unknown or other
  modified_base (8837)..(8842): a, c, g, t, unknown or other
  modified_base (10143)..(10145): a, c, g, t, unknown or other
tccttaattaannntcccttccagctctctgccccttttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaannnggtaccgagctcttacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatttctagctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgagtctagagatatcgaattcaagcttgtcgacagatctnnntaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttctgccgattataaggatgacgatgacaagctagccgcacaggttctcagaggcactgtgactgacttccctggatttgatgagcgggctgatgcagaaactcttcggaaggctatgaaaggcttgggcacagatgaggagagcatcctgactctgttgacatcccgaagtaatgctcagcgccaggaaatctctgcagcttttaagactctgtttggcagggatcttctggatgacctgaaatcagaactaactggaaaatttgaaaaattaattgtggctctgatgaaaccctctcggctttatgatgcttatgaactgaaacatgccttgaagggagctggaacaaatgaaaaagtactgacagaaattattgcttcaaggacacctgaagaactgagagccatcaaacaagtttatgaagaagaatatggctcaagcctggaagatgacgtggtgggggacacttcagggtactaccagcggatgttggtggttctccttcaggctaacagagaccctgatgctggaattgatgaagctcaagttgaacaagatgctcaggctttatttcaggctggagaacttaaatgggggacagatgaagaaaagtttatcaccatctttggaacacgaagtgtgtctcatttgagaaaggtgtttgacaagtacatgactatatcaggatttcaaattgaggaaaccattgaccgcgagacttctggcaatttagagcaactactccttgctgttgtgaaatctattcgaagtatacctgcctaccttgcagagaccctctattatgctatgaagggagctgggacagatgatcataccctcatcagagtcatggtttccaggagtgagattgatctgtttaacatcaggaaggagtttaggaagaattttgccacctctctttattccatgattaagggagatacatctggggactataagaaagctcttctgctgctctgtggagaagatgactaagctagccataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagactnnngtttaaacgaattcnnntataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatnnnttaattaannnnnnnnnncggcgcattaagcgcgcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatcgcggtctattcttttgatttataagggatgttgccgatttcggcctattggttaaaaaatgagctgatttaacaaaaattttaacaaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttnnnnnncaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgnnngga

SEQ ID NO: 9 - protein, 6 aa, Artificial Sequence
  Description of Artificial Sequence Synthetic 6xHis tag
HHHHHH